(19) United States
(12) Patent Application Publication
(10) Pub. No.: US 2005/0176740 A1
(43) Pub. Date: Aug. 11, 2005

(54) Cancer treatment method comprising administering an erb-family inhibitor and a raf and/or ras inhibitor

(76) Inventors: Neil Lee Spector, Durham, NC (US); Wenle Xia, Durham, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/510,542

(22) PCT Filed: Apr. 8, 2003
(86) PCT No.: PCT/US03/10747

Related U.S. Application Data
(60) Provisional application No. 60/370,807, filed on Apr. 8, 2002.

(51) Int. Cl. A61K 31/519

(52) U.S. Cl. 514/264.11

(57) ABSTRACT

The present invention relates to a method of treating cancer in a mammal and to pharmaceutical combinations useful in such treatment. In particular, the method relates to a cancer treatment method that includes administering an erb family inhibitor and a Raf and/or ras inhibitor to a mammal suffering from a cancer.

### BACKGROUND OF THE INVENTION

The present invention relates to a method of treating cancer in a mammal and to pharmaceutical combinations useful in such treatment. In particular, the method relates to a cancer treatment method that includes administering an erbB-2 and/or an EGFR inhibitor and a Raf inhibitor to a mammal suffering from a cancer, the cancer being characterized by ras oncogene overexpression.

Effective chemotherapy for cancer treatment is a continuing goal in the oncology field. Generally, cancer results from the deregulation of the normal processes that control cell division, differentiation and apoptotic cell death. Protein kinases play a critical role in these regulatory processes. One class of protein kinases currently under investigation is protein tyrosine kinases (PTKs). PTKs catalyze the phosphorylation of specific tyrosyl residues in various proteins involved in the regulation of cell growth and differentiation. (A. F. Wilks, Progress in Growth Factor Research, 1990, 2, 97-111; S. A. Courtneidge, Dev. Supp. 1, 1993, 57-64; J. A. Cooper, Semin. Cell Biol., 1994, 5 (6), 377-387; R. F. Paulson, Semin. Immunol., 1995, 7 (4), 267-277; A. C. Chan, Curr. Opin. Immunol., 1996, 8 (3), 394-401).

One of the most commonly studied pathways involving kinase regulation is cellular signaling from growth factor receptors at the cell surface to the nucleus (Crews and Erikson, 1993). One group of pathways, which have drawn interest is the mitogen activated protein kinase (MAPK) signaling pathways. These pathways are a cascade of kinases in which PTK growth factor receptors at the cell surface, for example, erbB family kinases, deliver signals from the cell surface by a phosphorylation mechanism, to other kinases such as Src tyrosine kinase, and the Raf, Mei; and Erk serine/threonine kinase families (Crews and Erikson, 1993; Ihle et al., 1994) which in turn continue the signal down the cascade to the cell nucleus (see FIG. 6). Each of these kinases is represented by several family members (Pelech and Sanghera, 1992) which play related, but functionally distinct roles. The loss of regulation of a growth factor signaling pathway is a frequent occurrence in cancer as well as other disease states.

The erbB family of type I receptor tyrosine kinases (erbB1 also known as EGFR: the epidermal growth factor receptor, erbB2, erbB3, and erbB4) are widely expressed in epithelial, mesenchymal, and neuronal tissues where they play a pivotal role in regulating cell proliferation, survival, and differentiation (Sibilia and Wagner, 1995; Threadgill et al., 1995). Overexpression of wild-type erbB2 or EGFR, or expression of constitutively activated receptor mutants, transform cells in vitro (Di Fiore et al., 1987; DiMarco et al., 1989; Hudziak et al., 1987; Qian et al., 1995). Overexpression of either EGFR or erbB2, or gene amplification as in the case of erbB2, correlates with a poorer clinical outcome in some breast cancers and a variety of other malignancies (Slamon et al, 1987; Slamon et al., 1989; Berchuck et al., 1990; Kern et al., 1990; Hynes and Stern, 1994; Bacus et al., 1994; Alimandi et al., 1995; Shoyab et al., 1989). Consequently, a great deal of attention has focused on developing therapeutics targeting either receptor through monoclonal antibodies or small molecule inhibitors.

With the exception of erbB3, all erbB receptor family members share a highly conserved cytoplasmic tyrosine kinase domain. For example, the tyrosine kinase domain of EGFR and erbB2 are 82% homologous based on amino acid sequence (Murali et al., 1996). Autophosphorylation of specific cytoplasmic tyrosine residues establishes binding sites for SH2 and phosphotyrosine-binding-domain containing proteins that in turn link to downstream effectors involved in cell proliferation (mitogen-activated protein kinases or MAPK) and survival (phosphatidylinositol 3-kinase/AKT) pathways (Hackel et al., 1999; Olayioye et al., 1998; Luttrell et al., 1994; Levkowitz et al., 1996; Klapper et al., 2000; Okano et al., 2000; Egan and Weinberg, 1993; Kavanaugh and Williams, 1994; Bjorge et al., 1990; Daly R J, 1999).

Raf protein kinases are key components of signal transduction pathways by which specific extracellular stimuli elicit precise cellular responses in mammalian cells. Activated cell surface receptors activate ras/rap proteins at the inner aspect of the plasma-membrane which in turn recruit and activate Raf proteins. Activated Raf proteins phosphorylate and activate the intracellular protein kinases MEK1 and MEK2. In turn, activated MEKs catalyse phosphorylation and activation of p42/p44 mitogen-activated protein kinase (MAPK). A variety of cytoplasmic and nuclear substrates of activated MAPK are known which directly or indirectly contribute to the cellular response to environmental change. Three distinct genes have been identified in mammals that encode Raf proteins; A-Raf, B-Raf and C-Raf (also known as Raf-1) and isoformic variants that result from differential splicing of mRNA are known.

Inhibitors of Raf kinases have been suggested for use in disruption of tumor cell growth and hence in the treatment of cancers, e.g. histiocytic lymphoma, lung adenocarcinoma, small cell lung cancer and pancreatic and breast carcinoma; also in the treatment and/or prophylaxis of disorders associated with neuronal degeneration resulting from ischemic events, including cerebral ischemia after cardiac arrest, stroke and multi-infarct dementia and also after cerebral ischemic events such as those resulting from head injury, surgery and/or during childbirth; also in chronic neurodegeneration such as Alzheimer's disease and Parkinson's disease; also in the treatment of pain, migraine and cardiac hypertrophy.

The kinase cRaf1 regulates cellular proliferation in two ways. The enzyme positively regulates cell division through the Raf/MEK/ERK protein kinase cascade. This activation is the result of cRaf1 catalyzed phosphorylation of the protein kinase, MEK1. MEK1 phosphorylates and activates the protein kinase ERK, which in turn phosphorylates and regulates transcription factors required for cell division (Avruch et al, TIBS; 1994 (19) 279-283). cRaf1 negatively regulates cell death by modulation of the activity of Bcl-2, a critical regulator of apoptosis. This regulation involves direct phosphorylation of Bcl-2 family members (Gajewski and Thompson, Cell: 1996 (87) 619-628). Both of these aspects of cRaf1 mediated regulation of cellular proliferation require the kinase activity of cRaf1.

cRaf1 is deregulated by events that are common in human cancer. For example ras genes are mutated with the following frequencies in the following representative primary human tumors: lung (adenocarcinoma), 300%; colon (adenocarcinoma), 50%; pancreatic carcinoma, 90%; seminoma, 40%; thyroid, 50% (McCormick, Ras oncogenes in Oncogenes and the molecular origins of cancer: 1989, 125-146). cRaf1 is also activated by deregulation of tyrosine kinases including, cSrc, ErbB2, EGFR, and bcr/abl. These events are associated with breast, colon and lung carcinomas and chronic myelogenous leukemia (Fearon, Genetic lesions in human cancer, in Molecular oncology; 1996, 143-178). In addition, Raf anti-sense literature teaches that the reduction of Raf protein levels correlates with a reduction in tumor growth rate in in vivo tumor mouse models. Inhibitors of the kinase activity of cRaf1 should therefore provide effective treatment for a wide variety of common human cancers.

The present inventors have noted that in certain tumors, which overexpress mutant ras, resistance to erbB-2 inhibitor therapy is conferred. Furthermore, therapy by administration of a Raf inhibitor, such as a cRaf-1 or bRaf inhibitor, appears to be ineffective. The present inventors have now recognized, that a combination of erb family and Raf inhibitors appears to be more effective than either therapy by itself. Accordingly, the present inventors have now discovered a new method of treating cancer using a novel pharmaceutical combination, which can selectively treat susceptible cancers. The present invention recognizes the use of Raf inhibitors, which can provide additional anti-neoplastic activity in patients where anti-neoplastic activity provided by erbB family inhibition is attenuated by increased ras/raf kinase activity. Specifically, the combination of a dual EGFR/erbB-2 inhibitor and a cRaf-1 and/or b-Raf inhibitor appears to effectively inhibit growth of such tumors.

### SUMMARY OF THE INVENTION

In a first aspect of the present invention, there is provided a method of treating a susceptible cancer in a mammal, comprising: administering to said mammal therapeutically effective amounts of (i) at least one erb family inhibitor and (ii) at least one Raf and/or ras inhibitor.

In a second aspect of the present invention, there is provided a method of treating a susceptible cancer in a mammal, comprising: administering to said mammal therapeutically effective amounts of (i) an erbB-2 inhibitor and (ii) a cRaf-1 inhibitor.

In a third aspect of the present invention, there is provided a method of treating a susceptible cancer in a mammal, comprising: administering to said mammal therapeutically effective amounts of (i) a compound of formula (I) 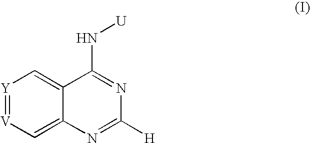
or a salt, solvate, physiologically functional derivative thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 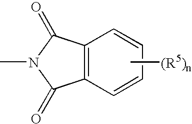
   wherein each R⁵ is independently selected from halogen, C₁₋₄ alkyl and C₁₋₄ alkoxy; and
n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N-(C₁₋₄ alkyl)carbamoyl, N,N-di(C₁₋₄ alkyl)carbamoyl, cyano, nitro and trifluoromethyl; and
(ii) a cRaf-1 inhibitor.

In a fourth aspect of the present invention, there is provided a method of treating a susceptible cancer in a mammal, comprising: administering to said mammal therapeutically effective amounts of (i) a compound of formula (II): 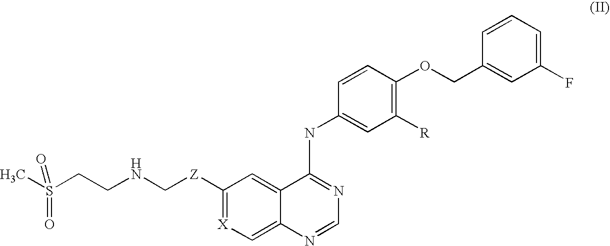
and salt or solvates thereof, wherein R is —Cl or —Br, X is CH, N, or CF, and Z is thiazole or furan; and
(ii) a cRaf-1 inhibitor.

In a fifth aspect of the present invention, there is provided a method of treating a susceptible cancer in a mammal, comprising: administering to said mammal therapeutically effective amounts of (i) a compound of formula (III): 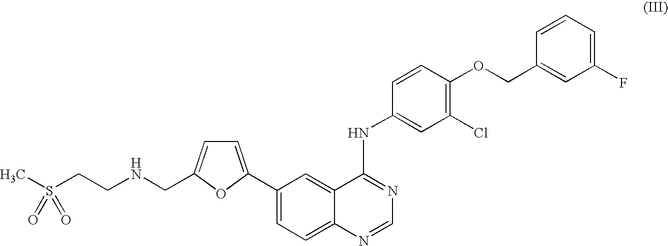
and salts or solvates thereof; and
(ii) a cRaf-1 inhibitor.

In a sixth aspect of the present invention, there is provided a cancer treatment combination, comprising: therapeutically effective amounts of (i) at least one erb family inhibitor and (ii) at least one Raf and/or ras inhibitor.

In a seventh aspect of the present invention, there is provided a cancer treatment combination, comprising: therapeutically effective amounts of (i) an erbB-2 inhibitor and (ii) a cRaf-1 inhibitor.

In an eighth aspect of the present invention, there is provided a cancer treatment combination, comprising: therapeutically effective amounts of (i) a compound of formula (I) 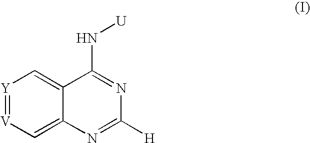
or a salt, solvate, or physiologically functional derivative thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 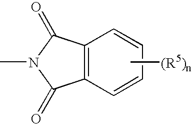
   wherein each R⁵ is independently selected from halogen, C₁₋₄ alkyl and C₁₋₄ alkoxy; and
n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₄ alkylcarbonyl, carboxy, carbamoyl, C₁₋₄alkoxycarbonyl, C₁₋₄ alkanoylamino, N-(C₁₋₄alkyl)carbamoyl, N,N-di(C₁₋₄ alkyl)carbamoyl, cyano, nitro and trifluoromethyl; and
(ii) a cRaf-1 inhibitor.

In a ninth aspect of the present invention, there is provided a cancer treatment combination, comprising: therapeutically effective amounts of (i) a compound of formula (II): 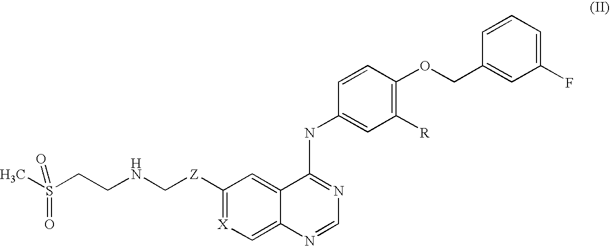
and salt or solvates thereof, wherein R is —Cl or —Br, X is CH, N, or CF, and Z is thiazole or furan; and
(ii) a cRaf-1 inhibitor.

In an tenth aspect of the present invention, there is provided a cancer treatment combination, comprising: therapeutically effective amounts of (i) a compound of formula (III): 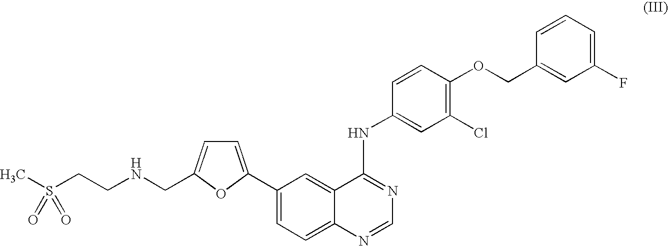
and salts or solvates thereof; and
(ii) a cRaf-1 inhibitor.

In an eleventh aspect of the present invention, there is provided a cancer treatment combination, comprising: therapeutically effective amounts of (i) at least one erb family inhibitor and (ii) at least one Raf and/or ras inhibitor for use in therapy.

In a twelfth aspect of the present invention, there is provided a cancer treatment combination, comprising: therapeutically effective amounts of (i) an erbB-2 inhibitor and (ii) a cRaf-1 inhibitor for use in therapy.

In a thirteenth aspect of the present invention, there is provided use of a cancer treatment combination, comprising: therapeutically effective amounts of (i) at least one erb family inhibitor and (ii) at least one Raf and/or ras inhibitor in the preparation of a medicament for use in the treatment of a susceptible cancer.

In a fourteenth aspect of the present invention, there is provided use of a cancer treatment combination, comprising: therapeutically effective amounts of (i) an EGFR/erbB-2 inhibitor and (ii) a cRaf-1 inhibitor in the preparation of a medicament for use in the treatment of a susceptible cancer.

In another aspect of the present invention, there is provided a method of treating a susceptible cancer in a mammal, comprising: administering to said mammal therapeutically effective amounts of (i) an erbB-2 inhibitor and (ii) a bRaf inhibitor.

In another aspect of the present invention, there is provided a method of treating a susceptible cancer in a mammal, comprising: administering to said mammal therapeutically effective amounts of (i) a compound of formula (I) 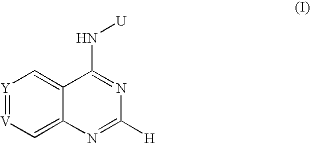
or a salt, solvate, physiologically functional derivative thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 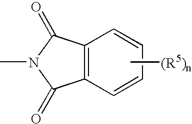
   wherein each R⁵ is independently selected from halogen, C₁₋₄alkyl and C₁₋₄ alkoxy; and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N-(C₁₋₄ alkyl)carbamoyl, N,N-di(C₁₋₄ alkyl)carbamoyl, cyano, nitro and trifluoromethyl; and
(ii) a bRaf inhibitor.

In another aspect of the present invention, there is provided a method of treating a susceptible cancer in a mammal, comprising: administering to said mammal therapeutically effective amounts of (i) a compound of formula (II): 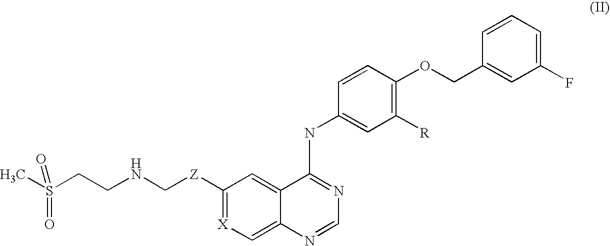
and salt or solvates thereof, wherein R is —Cl or —Br, X is CH, N, or CF, and Z is thiazole or furan; and
(ii) a bRaf inhibitor.

In still another aspect of the present invention, there is provided a method of treating a susceptible cancer in a mammal, comprising: administering to said mammal therapeutically effective amounts of (i) a compound of formula (III): 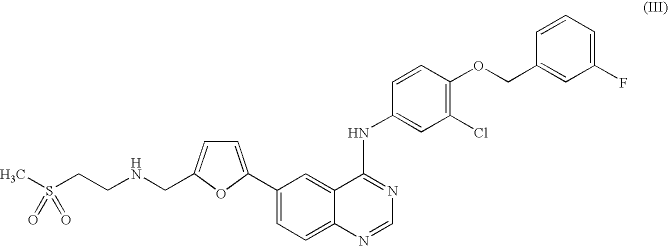
and salts or solvates thereof; and
(ii) a bRaf inhibitor.

In a further aspect of the present invention, there is provided a cancer treatment combination, comprising: therapeutically effective amounts of (i) an erbB-2 inhibitor and (ii) a bRaf inhibitor.

In another aspect of the present invention, there is provided a cancer treatment combination, comprising: therapeutically effective amounts of (i) a compound of formula (I) 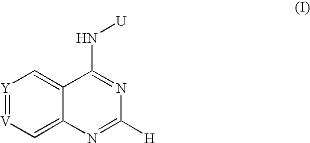
or a salt, solvate, or physiologically functional derivative thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 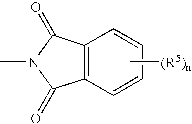
   wherein each R⁵ is independently selected from halogen, C₁₋₄alkyl and C₁₋₄ alkoxy; and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; and
(ii) a bRaf inhibitor.

In a further aspect of the present invention, there is provided a cancer treatment combination, comprising: therapeutically effective amounts of (i) a compound of formula (II): 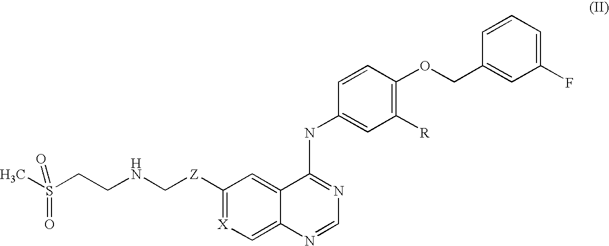
and salt or solvates thereof, wherein R is —Cl or —Br, X is CH, N, or CF, and Z is thiazole or furan; and
(ii) a bRaf-1 inhibitor.

In another aspect of the present invention, there is provided a cancer treatment combination, comprising: therapeutically effective amounts of (i) a compound of formula (III): 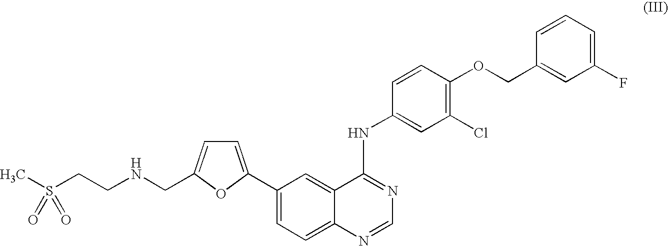
and salts or solvates thereof; and
(ii) a bRaf inhibitor.

In a further aspect of the present invention, there is provided a cancer treatment combination, comprising: therapeutically effective amounts of (i) an erbB-2 inhibitor and (ii) a bRaf inhibitor for use in therapy.

In a still further aspect of the present invention, there is provided use of a cancer treatment combination, comprising: therapeutically effective amounts of (i) an EGFR/erbB-2 inhibitor and (ii) a bRaf inhibitor in the preparation of a medicament for use in the treatment of a susceptible cancer.

### BRIEF DESCRIPTION OF THE DRAWINGS

FIG. 1 depicts a bar chart illustrating down regulation of erbB-2 in an Hb4a cell line transfected with Ha-(Val. 12)-ras.

FIG. 2 depicts a Western Blot study showing the effect of GW2016 on Ha-(Val12)-ras or erbB-2 transfected HB4a cell lines.

FIG. 3 depicts a Western Blot study showing the effect of Ha-(Val12)-ras transfection on the inhibitory effects of GW2016 on MAPK activation in HB4a cells.

FIG. 4 depicts a bar chart showing cell mortality from exposure to GW2016 or GW5074 or GW2016+GW5074.

FIG. 5 depicts a bar chart showing a summary of a cell cycle distribution analyses of cells exposed to GW2016 or GW5074 or GW2016+GW5074.

FIG. 6 depicts postulated HER-2 (erbB2) signal transduction pathways.

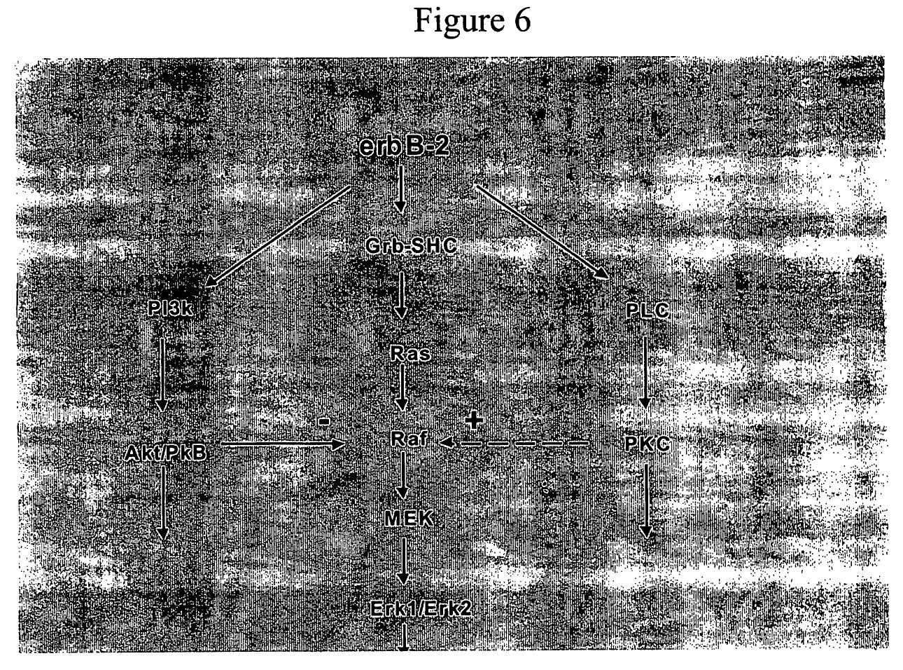

FIG. 7 depicts a bar chart showing PANC-1 Cell Proliferation and Viability by Mitochondrial Dehydrogenase Activity (O.D. 485) after 72 hour treatment with GW2016 and bRaf inhibitors of Examples 13 (B1) and 14(B2).

FIG. 8 depicts a bar chart showing PANC-1 Cell Proliferation and Viability by Mitochondrial Dehydrogenase Activity (O.D. 485) after 96 hour treatment with GW2016 and bRaf inhibitors of Examples 13 (B1) and 14(B2).

FIG. 9 depicts a bar chart showing CFPAC-1 Cell Proliferation and Viability by Mitochondrial Dehydrogenase Activity (O.D. 485) after 72 hour treatment with GW2016 and bRaf inhibitors of Examples 13 (B1) and 14(B2).

FIG. 10 depicts a bar chart showing CFPAC-1 Cell Proliferation and Viability by Mitochondrial Dehydrogenase Activity (O.D. 485) after 96 hour treatment with GW2016 and bRaf inhibitors of Examples 13 (B1) and 14(B2).

### DETAILED DESCRIPTION OF THE INVENTION

As used herein the term “neoplasm” refers to an abnormal growth of cells or tissue and is understood to include benign, i.e., non-cancerous growths, and malignant, i.e., cancerous growths. The term “neoplastic” means of or related to a neoplasm.

As used herein the term “agent” is understood to mean a substance that produces a desired effect in a tissue, system, animal, mammal, human, or other subject. Accordingly, the term “anti-neoplastic agent” is understood to mean a substance producing an anti-neoplastic effect in a tissue, system, animal, mammal, human, or other subject. It is also to be understood that an “agent” may be a single compound or a combination or composition of two or more compounds.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the terms “Cₓ-C_{y}” or “C_{x-y}” where x and y represent an integer value refer to the number of carbon atoms in a particular chemical term to which it is attached. For instance, the term “C₁-C₄ alkyl” or “C₁₋₄ alkyl” refers to an alkyl group, as defined herein, containing at least 1, and at most 4 carbon atoms.

As used herein, the term “alkyl” refers to a straight or branched chain hydrocarbon radical having from one to twelve carbon atoms, optionally substituted with substituents selected from the group consisting of C₁-C₆ alkyl, C₁-C₆ hydroxyalkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aryl, aryloxy, heteroaryl, aminosulfonyl optionally substituted by alkyl, nitro, cyano, halogen, or C₁-C₆ perfluoroalkyl, multiple degrees of substitution being allowed. Examples of “alkyl” as used herein include, but are not limited to, methyl, ethyl, n-propyl, isopropyl, n-butyl, isobutyl, t-butyl, n-pentyl, isopentyl, and the like.

As used herein, the term “alkylene” refers to a straight or branched chain divalent hydrocarbon radical having from one to ten carbon atoms, optionally substituted with substituents selected from the group which includes C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen and C₁-C₆ perfluoroalkyl, multiple degrees of substitution being allowed. Examples of “alkylene” as used herein include, but are not limited to, methylene, ethylene, n-propylene, n-butylene, and the like.

As used herein, the term “alkenyl” refers to a hydrocarbon radical having from two to ten carbons and at least one carbon-carbon double bond, optionally substituted with substituents selected from the group which includes C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen and C₁-C₆ perfluoroalkyl, multiple degrees of substitution being allowed. Examples of “alkenyl” as used herein include, ethenyl, propenyl, 1-butenyl, 2-butenyl, and isobutenyl.

As used herein, the term “alkynyl” refers to a hydrocarbon radical having from two to ten carbons and at least one carbon-carbon triple bond, optionally substituted with substituents selected from the group which includes C₁-C₆ alkyl, C₁-C₆ alkoxy, C₁-C₆ alkylsulfanyl, C₁-C₆ alkylsulfenyl, C₁-C₆ alkylsulfonyl, oxo, aryl, hydroxy, mercapto, amino optionally substituted by alkyl, carboxy, carbamoyl optionally substituted by alkyl, aminosulfonyl optionally substituted by alkyl, nitro, cyano, halogen and C₁-C₆ perfluoroalkyl, multiple degrees of substitution being allowed. Examples of “alkynyl” as used herein, include but are not limited to acetylenyl, 1-propynyl, 1-butynyl, 2-butynyl, 1-pentynyl, and 1-hexynyl.

As used herein, the term “halogen” refers to fluorine (F), chlorine (Cl), bromine (Br), or iodine (I) and the term “halo” refers to the halogen radicals fluoro (—F), chloro (—Cl), bromo (—Br), and iodo (—I).

As used herein, the term “haloalkyl” refers to an alkyl group, as defined above, substituted with at least one halo group, halo being as defined herein. Examples of such branched or straight chained haloalkyl groups useful in the present invention include, but are not limited to, methyl, ethyl, propyl, isopropyl, isobutyl and n-butyl substituted independently with one or more halos, e.g., fluoro, chloro, bromo and iodo.

As used herein, the term “cycloalkyl” refers to a non-aromatic cyclic hydrocarbon ring, which optionally includes a C₁-C₆ alkyl linker through which it may be attached. Exemplary “cycloalkyl” groups useful in the present invention include, but are not limited to, cyclopropyl, cyclobutyl, cyclopentyl, cyclohexyl and cycloheptyl.

As used herein, the term “heterocyclic” or the term “heterocyclyl” refers to a three to twelve-membered non-aromatic heterocyclic ring, being saturated or having one or more degrees of unsaturation, containing one or more heteroatom substitutions selected from S, S(O), S(O)₂, O, or N, optionally substituted with substituents selected from the group consisting of C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or C₁-C₆ perfluoroalkyl, multiple degrees of substitution being allowed. Such a ring may be optionally fused to one or more other “heterocyclic” ring(s) or cycloalkyl ring(s). Examples of “heterocyclic” moieties include, but are not limited to, tetrahydrofuran, pyran, 1,4-dioxane, 1,3-dioxane, piperidine, piperazine, 2,4-piperazinedione, pyrrolidine, imidazolidine, pyrazolidine, morpholine, thiomorpholine, tetrahydrothiopyran, tetrahydrothiophene, and the like.

As used herein, the term “aryl” refers to an optionally substituted benzene ring or to an optionally substituted benzene ring system fused to one or more optionally substituted benzene rings to form, for example, anthracene, phenanthrene, or napthalene ring systems. Exemplary optional substituents include C₁-C₆ alkyl, C₁-C₆ alkoxy, C₁-C₆ haloalkyl, C₁-C₆ haloalkoxy, C₁-C₆ alkylsulfanyl, C₁-C₆ alkylsulfenyl, C₁-C₆ alkylsulfonyl, C₁-C₆ alkylsulfonylamino, arylsulfonoamino, alkylcarboxy, alkylcarboxyamide, oxo, hydroxy, mercapto, amino optionally substituted by alkyl or acyl, carboxy, tetrazolyl, carbamoyl optionally substituted by alkyl, aryl, or heteroaryl, aminosulfonyl optionally substituted by alkyl, acyl, aroyl, aroylamino, heteroaroyl, acyloxy, aroyloxy, heteroaroyloxy, alkoxycarbonyl, nitro, cyano, halogen, heteroaryl, heterocyclyl, aryl optionally substituted with aryl, halogen, C₁-C₆alkyl, C₁-C₆ haloalkyl, or C₁-C₆ alkylsulfonyl, ureido, arylurea, alkylurea, cycloalkylurea, alkylthiourea, aryloxy, or aralkoxy, multiple degrees of substitution being allowed. Examples of “aryl” groups include, but are not limited to, phenyl, 2-naphthyl, 1-naphthyl, biphenyl, as well as substituted derivatives thereof.

As used herein, the term “aralkyl” refers to an aryl or heteroaryl group, as defined herein, attached through a C₁-C₃ alkylene linker, wherein the C₁-C₃ alkylene is as defined herein. Examples of “aralkyl” include, but are not limited to, benzyl, phenylpropyl, 2-pyridylmethyl, 3-isoxazolylmethyl, 5-methyl, 3-isoxazolylmethyl, and 2-imidazoyly ethyl.

As used herein, the term “heteroaryl” refers to a monocyclic five to seven membered aromatic ring, or to a fused bicyclic or tricyclic aromatic ring system comprising two of such monocyclic five to seven membered aromatic rings. These heteroaryl rings contain one or more nitrogen, sulfur, and/or oxygen heteroatoms, where N-oxides and sulfur oxides and dioxides are permissible heteroatom substitutions and may be optionally substituted with up to three members selected from a group consisting of C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, C₁-C₆ perfluoroalkyl, heteroaryl, or aryl, multiple degrees of substitution being allowed. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinolinyl, isoquinolinyl, benzofuranyl, benzothiophenyl, indolyl, indazolyl, and substituted versions thereof.

As used herein, the term “alkoxy” refers to the group RₐO—, where Rₐ is alkyl as defined above. Exemplary alkoxy groups useful in the present invention include, but are not limited to, methoxy, ethoxy, n-propoxy, isopropoxy, n-butoxy, and t-butoxy.

As used herein, the term “amino” refers to the group —NH₂.

As used herein the term “alkylamino” refers to the group —NHRₐ wherein Rₐ is alkyl as defined above.

As used herein the term “arylamino” refers to the group —NHRₐ wherein Rₐ is aryl as defined above.

As used herein the term “aralkylamino” refers to the group —NHRₐ wherein Rₐ is an aralkyl group as defined above.

As used herein the term “aralkoxy” refers to the group R_{b}RₐO—, where Rₐ is alkyl and R_{b} is aryl or heteroaryl all as defined above.

As used herein the term “aryloxy” refers to the group RₐO—, where Rₐ is aryl or heteroaryl both as defined above.

As used herein the term “ureido” refers to the group —NHC(O)NH₂.

As used herein, the term “arylurea” refers to the group —NHC(O)NHRₐ wherein Rₐ is aryl as defined above.

As used herein, the term “arylthiourea” refers to the group —NHC(S)NHRₐ wherein Rₐ is aryl as defined above.

As used herein, the term “alkylurea” refers to the group —NHC(O)NHRₐ wherein Rₐ is alkyl as defined above.

As used herein, the term “cycloalkylurea” refers to the group —NHC(O)NHRₐ wherein Rₐ is cycloalkyl as defined above.

As used herein, the term “cycloalkoxy” refers to the group RₐO—, where Rₐ is cycloalkyl as defined above. Exemplary cycloalkoxy groups useful in the present invention include, but are not limited to, cyclobutoxy, and cyclopentoxy.

As used herein, the term “haloalkoxy” refers to the group RₐO—, where Rₐ is haloalkyl as defined above. Exemplary haloalkoxy groups useful in the present invention include, but are not limited to, trifluoromethoxy.

As used herein, the terms “alkylsulfanyl” and “alkylthio” mean the same and refer to the group RₐS—, where Rₐ is alkyl as defined above.

As used herein, the term “haloalkylsulfanyl” refers to the group RₐS—, where Rₐ is haloalkyl as defined above.

As used herein, the term “alkylsulfenyl” refers to the group RₐS(O)—, where Rₐ is alkyl as defined above.

As used herein, the term “alkylsulfonyl” refers to the group RₐS(O)₂—, where Rₐ is alkyl as defined above.

As used herein, the term “alkylsulfonylamino” refers to the group —NHS(O)₂Rₐ wherein Rₐ is alkyl as defined above.

As used herein, the term “arylsulfonylamino” refers to the group —NHS(O)₂Rₐ wherein Rₐ is aryl as defined above.

As used herein, the term “alkylcarboxyamide” refers to the group —NHC(O)Rₐ wherein Rₐ is alkyl, amino, or amino substituted with alkyl, aryl or heteroaryl as described above.

As used herein, the term “oxo” refers to the group ═O.

As used herein, the term “mercapto” refers to the group —SH.

As used herein, the term “carboxy” refers to the group —C(O)OH.

As used herein, the term “cyano” refers to the group —CN.

As used herein the term “cyanoalkyl” refers to the group —CNRₐ, wherein Rₐ is alkyl as defined above. Exemplary “cyanoalkyl” groups useful in the present invention include, but are not limited to, cyanomethyl, cyanoethyl, and cyanoisopropyl.

As used herein, the term “aminosulfonyl” refers to the group —S(O)₂NH₂.

As used herein, the term “carbamoyl” refers to the group —C(O)NH₂.

As used herein, the term “sulfanyl” shall refer to the group —S—.

As used herein, the term “sulfenyl” shall refer to the group —S(O)—.

As used herein, the term “sulfonyl” shall refer to the group —S(O)₂— or —SO₂—.

As used herein, the terms ‘acyl’ and “alkylcarbonyl” are the same and refer to the group RₐC(O)—, where Rₐ is alkyl, cycloalkyl, or heterocyclyl as defined herein.

As used herein, the term “alkanoylamino” refers to the group RₐC(O)NH—, where Rₐ is alkyl as defined herein.

As used herein, the term “aroyl” refers to the group RₐC(O)—, where Rₐ is aryl as defined herein.

As used herein, the term “aroylamino” refers to the group RₐC(O)NH—, where Rₐ is aryl as defined herein.

As used herein, the term “heteroaroyl” refers to the group RₐC(O)—, where Rₐ is heteroaryl as defined herein.

As used herein, the term “alkoxycarbonyl” refers to the group RₐOC(O)—, where Rₐ is alkyl as defined herein.

As used herein, the term “acyloxy” refers to the group RₐC(O)O—, where Rₐ is alkyl, cycloalkyl, or heterocyclyl as defined herein.

As used herein, the term “aroyloxy” refers to the group RₐC(O)O—, where Rₐ is aryl as defined herein.

As used herein, the term “heteroaroyloxy” refers to the group RₐC(O)O—, where R^{a} is heteroaryl as defined herein.

As used herein, the term “optionally” means that the subsequently described event(s) may or may not occur, and includes both event(s), which occur, and events that do not occur.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compounds formulae (I), (I′), (I″), (II), (III), (III′), (III″), or (IV) or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

As used herein, the term “substituted” refers to substitution with the named substituent or substituents, multiple degrees of substitution being allowed unless otherwise stated.

Certain of the compounds described herein may contain one or more chiral atoms, or may otherwise be capable of existing as two enantiomers. The compounds of this invention include mixtures of enantiomers as well as purified enantiomers or enantiomerically enriched mixtures. Also included within the scope of the invention are the individual isomers of the compounds represented by formulae (I), (I′), (I″), (II), (III), (III′), (III″), or (IV) as well as any wholly or partially equilibrated mixtures thereof. The present invention also covers the individual isomers of the compounds represented by the formulas above as mixtures with isomers thereof in which one or more chiral centers are inverted. Also, it is understood that any tautomers and mixtures of tautomers of the compounds of formulae formulae (I), (I′), (I″), (II), (III), (III′), (III″), or formula (IV) are included within the scope of the compounds of formulae (I), (I′), (I″), (II), (III), (III′), (III″), or formula (IV).

As recited above, in one embodiment a method of treating cancer is provided which includes administering a therapeutically effective amount of at least one erb family inhibitor and at least one Raf and/or ras inhibitor. Preferably the erb family inhibitor is a dual inhibitor of erbB-2 and EGFR and the Raf and/or ras inhibitor is a cRaf-1 inhibitor or a bRaf inhibitor.

Generally, any EGFR/erbB-2 inhibitor, that is any pharmaceutical agent having specific erbB-2 and/or EGFR inhibitor activity may be utilized in the present invention. Such erbB-2/EGFR inhibitors are described, for instance, in U.S. Pat. Nos. 5,773,476; 5,789,427; 6,103,728; 6,169,091; 6,174,889; and 6,207,669; and International Patent Applications WO 95/24190; WO 98/0234; WO 99/35146; WO 01/04111; and WO 02/02552 which patents and patent applications are herein incorporated by reference to the extent of their disclosure of erbB-2 and/or EGFR inhibitor compounds as well as methods of making the same.

One class of dual EGFR/erbB-2 inhibitor compounds that may be usefully employed in the present invention includes compounds of the Formula I: 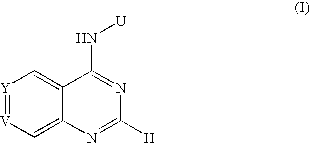
or a salt, solvate, or physiologically functional derivative thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁alkoxy groups;
R² is selected from the group comprising hydrogen, halo, hydroxy, C₁₋₄ alkyl, C₁₋₄ alkoxy, C₁₋₄ alkylamino and di[C-4 alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 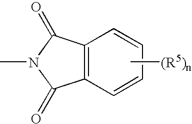
   wherein each R⁵ is independently selected from halogen, C₁₋₄alkyl and C₁₋₄ alkoxy; and
n is 0 to 3; and
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N-(C₁₋₄ alkyl)carbamoyl, N,N-di(C₁₋₄ alkyl)carbamoyl, cyano, nitro and trifluoromethyl.

The definitions for Y and V thus give rise to two possible basic ring systems for the compounds of formula (I). In particular the compounds may contain the following basic ring systems: quinazolines (1) and pyrido-pyrimidines (2):

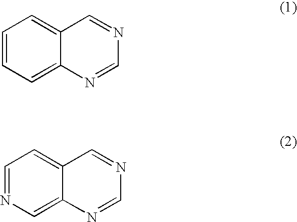

In a preferred embodiment, the ring system is ring (1).

Suitable values for the various groups listed above within the definitions for R¹, R², R⁴ and R⁵ are as follows:
halo is, for example, fluoro, chloro, bromo or iodo; preferably it is fluoro, chloro or bromo, more preferably fluoro or chloro;
C₁₋₄ alkyl is, for example, methyl, ethyl, propyl, isopropyl, butyl, isobutyl, sec-butyl or tert-butyl; preferably it is methyl, ethyl, propyl, isopropyl or butyl, more preferably methyl;
C₂₋₄ alkenyl is, for example, ethenyl, prop-1-enyl or prop-2-enyl; preferably ethenyl;
C₂₋₄ alkynyl is, for example, ethynyl, prop-1-ynyl or prop-2-ynyl; preferably ethynyl;
C₁₋₄ alkoxy is, for example, methoxy, ethoxy, n-propoxy, isopropoxy, n-butoxy, isobutoxy, sec-butoxy or tert-butoxy; preferably methoxy, ethoxy, propoxy, isopropoxy or butoxy; more preferably methoxy;
C₁₋₄ alkylamino is, for example, methylamino, ethylamino or propylamino; preferably methylamino;
di[C₁₋₄ alkyl]amino is, for example, dimethylamino, diethylamino, N-methyl-N-ethylamino or dipropylamino; preferably dimethylamino;
C₁₋₄ alkylthio is, for example, methylthio, ethylthio, propylthio or isopropylthio, preferably methylthio;
C₁₋₄ alkylsulphinyl is, for example, methylsulphinyl, ethylsulphinyl, propylsulphinyl or isopropylsulphinyl, preferably methylsulphinyl;
C₁₋₄ alkylsulphonyl is, for example, methanesulphonyl, ethylsulphonyl, propylsulphonyl or isopropylsulphonyl, preferably methanesulphonyl;
C₁₋₄ alkylcarbonyl is, for example methylcarbonyl, ethylcarbonyl or propylcarbonyl;
C₁₋₄ alkoxycarbonyl is, for example, methoxycarbonyl, ethoxycarbonyl, propoxycarbonyl, butoxycarbonyl or tert-butoxycarbonyl;
C₁₋₄ alkanoylamino (where the number of carbon atoms includes the CO functionality) is, for example, formamido, acetamido, propionamido or butyramido;
N—(C₁₋₄ alkyl)carbamoyl is, for example, N-methylcarbamoyl or N-ethylcarbamoyl; and
N,N-di(C₁₋₄ alkyl)carbamoyl is, for example, N,N-dimethylcarbamoyl, N-methyl-N-ethylcarbamoyl or N,N-diethylcarbamoyl.

In a preferred embodiment, Y is CR¹ and V is CR² (ring system (1) above).

In another embodiment, Y is CR¹ and V is N (ring system (2) above).

In one embodiment, R² represents hydrogen or C₁₋₄ alkoxy.

In a preferred embodiment, R² represents hydrogen or methoxy.

In another preferred embodiment, R² represents halo; more preferred R² is fluoro.

In a preferred embodiment, the group Ar is substituted by one halo, C₁₋₄ alkyl or C₁₋₄ alkoxy group.

In a more preferred embodiment, the group Ar is substituted by a C₁₋₄ alkyl group.

In another preferred embodiment, the group Ar does not carry any optional substituents.

In a further more preferred embodiment, Ar represents furan, phenyl or thiazole, each of which may optionally be substituted as indicated above.

In a further more preferred embodiment, Ar represents furan or thiazole, each of which may optionally be substituted as indicated above.

In a most preferred embodiment, Ar represents unsubstituted furan or thiazole.

The side chain CH₃SO₂CH₂CH₂NHCH₂ may be linked to any suitable position of the group Ar. Similarly, the group R¹ may be linked to the carbon atom carrying it from any suitable position of the group Ar.

In a preferred embodiment, when Ar represents furan the side chain CH₃SO₂CH₂CH₂NHCH₂ is in the 4-position of the furan ring and the link to the carbon atom carrying the group R¹ is from the 2-position of the furan ring.

In another preferred embodiment, when Ar represents furan the side chain CH₃SO₂CH₂CH₂NHCH₂ is in the 3-position of the furan ring and the link to the carbon atom carrying the group R¹ is from the 2-position of the furan ring.

In a most preferred embodiment, when Ar represents furan the side chain CH₃SO₂CH₂CH₂NHCH₂ is in the 5-position of the furan ring and the link to the carbon atom carrying the group R¹ is from the 2-position of the furan ring.

In a further most preferred embodiment, when Ar represents thiazole the side chain CH₃SO₂CH₂CH₂NHCH₂ is in the 2-position of the thiazole ring and the link to the carbon atom carrying the group R¹ is from the 4-position of the thiazole ring.

The R³ and R⁴ groups may be bound to the ring system U by either a carbon atom or a heteroatom of the ring system. The ring system itself may be bound to the bridging NH group by a carbon atom or a heteroatom but is preferably bound by a carbon atom. The R³ and R⁴ groups may be bound to either ring when U represents a bicyclic ring system, but these groups are preferably bound to the ring which is not bound to the bridging NH group in such a case.

In a preferred embodiment U, represents a phenyl, indolyl, or 1H-indazolyl group substituted by an R³ group and optionally substituted by at least one independently selected R⁴ group.

In a more preferred embodiment, U represents a phenyl or 1H-indazolyl group substituted by an R³ group and optionally substituted by at least one independently selected R⁴ group.

In a more preferred embodiment, where U represents a phenyl group the group R³ is in the para-position relative to the bond from U to the linking NH group.

In a further more preferred embodiment, where U represents a 1H-indazolyl group the group R³ is in the 1-position of the indazolyl group.

In a preferred embodiment, R³ represents benzyl, pyridylmethyl, phenoxy, benzyloxy, halo-, dihalo- and trihalobenzyloxy and benzenesulphonyl.

In a further preferred embodiment, R³ represents trihalomethylbenzyloxy.

In a further preferred embodiment, R³ represents a group of formula 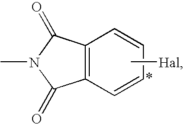
wherein Hal is Br or Cl, particularly Cl, more especially wherein the Hal substituent is in the position marked with a star in the ring as shown.

In a more preferred embodiment, R³ represents benzyloxy, fluorobenzyloxy (especially 3-fluorobenzyloxy), benzyl, phenoxy and benzenesulphonyl.

In a further more preferred, embodiment R³ represents bromobenzyloxy (especially 3-bromobenzyloxy) and trifluoromethylbenzyloxy.

In a further preferred embodiment, the ring U is not substituted by an R⁴ group; in an especially preferred embodiment U is phenyl or indazolyl unsubstituted by an R⁴ group.

In a further preferred embodiment, the ring U is substituted by an R⁴ group selected from halo or C₁₋₄ alkoxy; especially chloro, fluoro or methoxy.

In a more preferred embodiment, the ring U is substituted by an R⁴ group wherein R⁴ represents halo, especially 3-fluoro.

In another preferred embodiment, U together with R⁴ represents methoxyphenyl, fluorophenyl, trifluoromethylphenyl or chlorophenyl.

In a further preferred embodiment, U together with R⁴ represents methoxyphenyl or fluorophenyl.

In another preferred embodiment, the group U together with the substituent(s) R³ and R⁴ represents benzyloxyphenyl, (fluorobenzyloxy)phenyl, (benzenesulphonyl)phenyl, benzylindazolyl or phenoxyphenyl.

In still another preferred embodiment, the group U together with the substituent(s) R³ and R⁴ represents benzyloxyphenyl, (3-fluorobenzyloxy)phenyl, (benzenesulphonyl)phenyl or benzylindazolyl.

In another preferred embodiment, the group U together with the substituent(s) R³ and R⁴ represents (3-bromobenzyloxy)phenyl, (3-trifluoromethylbenzyloxy)phenyl, or (3-fluorobenzyloxy)-3-methoxyphenyl.

In a more preferred embodiment, the group U together with the substituent(s) R³ and R⁴ represents 3-fluorobenzyloxy-3-chlorophenyl, benzyloxy-3-chlorophenyl, benzyloxy-3-trifluoromethylphenyl, (benzyloxy)-3-fluorophenyl, (3-fluorobenzyloxy)-3-fluorophenyl or (3-fluorobenzyl)indazolyl.

In another preferred embodiment the group U together with the substituent(s) R³ and R⁴ represents benzyloxyphenyl or (3-fluorobenzyloxy)phenyl.

In a preferred embodiment, there is provided a compound of formula (I) or a salt, solvate, or physiologically functional derivative thereof wherein V is CR², wherein R² is hydrogen, halo (especially fluoro) or C₁₋₄ alkoxy (especially methoxy); Y is CR¹ wherein R¹ is as defined above in which Ar is unsubstituted phenyl, furan or thiazole; U is phenyl or indazole; R³ is benzyl, fluorobenzyl, benzyloxy, fluorobenzyloxy, bromobenzyloxy, trifluoromethylbenzyloxy, phenoxy or benzenesulphonyl; and R⁴ is not present or is halo (especially chloro or fluoro), or methoxy.

In another preferred embodiment, there is provided a compound of formula (I) or a salt, solvate, or physiologically functional derivative thereof wherein V is CR², wherein R² is hydrogen, halo (especially fluoro) or C₁₋₄ alkoxy (especially methoxy); Y is CR¹ wherein R¹ is as defined above in which Ar is unsubstituted furan or thiazole; U is phenyl; R³ is benzyloxy, fluorobenzyloxy or benzenesulphonyl; and R⁴ is not present or is halo (especially chloro or fluoro), or methoxy.

In a preferred embodiment, there is provided a compound of formula (I) or a salt, solvate, or physiologically functional derivative thereof wherein V is CR², wherein R² is hydrogen, halo (especially fluoro) or C₁₋₄ alkoxy (especially methoxy); Y is CR¹ wherein R¹ is as defined above in which Ar is unsubstituted furan or thiazole; U is indazole; R³ is benzyl or fluorobenzyl; and R⁴ is not present.

In a further preferred embodiment, there is provided a compound of formula (I) or a salt, solvate, or physiologically functional derivative thereof wherein Y is CR², wherein R² is hydrogen, halo (especially fluoro) or C₁₋₄ alkoxy (especially methoxy); V is CR¹ wherein R¹ is as defined above in which Ar is unsubstituted phenyl, furan or thiazole; U is phenyl or indazole; R³ is benzyl, fluorobenzyl, benzyloxy, fluorobenzyloxy, bromobenzyloxy, trifluoromethylbenzyloxy, phenoxy or benzenesulphonyl; and R⁴ is not present or is halo (especially chloro or fluoro), or methoxy.

In a another preferred embodiment, there is provided a compound of formula (I) or a salt, solvate, or physiologically functional derivative thereof wherein Y is CR², wherein R² is hydrogen, halo (especially fluoro) or C₁₋₄ alkoxy (especially methoxy); V is CR¹ wherein R¹ is as defined above in which Ar is unsubstituted furan or thiazole; U is phenyl; R³ is benzyloxy, fluorobenzyloxy or benzenesulphonyl; and R⁴ is not present or is halo (especially chloro or fluoro), or methoxy.

In another preferred embodiment, there is provided a compound of formula (I) or a salt, solvate, or physiologically functional derivative thereof wherein Y is CR², where; R² is hydrogen, halo (especially fluoro) or C₁₋₄ alkoxy (especially methoxy); V is CR¹ wherein R¹ is as defined above in which Ar is unsubstituted furan or thiazole; U is indazole; R³ is benzyl or fluorobenzyl; and R⁴ is not present.

In another preferred embodiment, there is provided a compound of formula (I) or a salt, solvate, or physiologically functional derivative thereof wherein Y is CR², wherein R² is hydrogen, halo (especially fluoro) or C₁₋₄ alkoxy (especially methoxy); V is CR¹ wherein R¹ is as defined above in which Ar is unsubstituted furan or thiazole; U is phenyl; R³ is phenoxy; and R⁴ is not present.

In another more preferred embodiment, there is provided a compound of formula (I) or a salt, solvate, or physiologically functional derivative thereof wherein V is N; Y is CR¹ wherein R¹ is as defined above in which Ar is unsubstituted phenyl, furan or thiazole; U is phenyl or indazole; R³ is benzyl, fluorobenzyl, benzyloxy, fluorobenzyloxy, bromobenzyloxy, trifluoromethylbenzyloxy, phenoxy or benzenesulphonyl; and R⁴ is not present or is halo (especially chloro or fluoro), or methoxy.

In another most preferred embodiment, there is provided a compound of formula (I) or a salt, solvate, or physiologically functional derivative thereof wherein V is N, Y is CR¹ wherein R¹ is as defined above in which Ar is unsubstituted furan or thiazole; U is phenyl; R³ is benzyloxy, fluorobenzyloxy or benzenesulphonyl; and R⁴ is not present or is halo (especially chloro or fluoro), or methoxy.

In another most preferred embodiment, there is provided a compound of formula (I) or a salt, solvate, or physiologically functional derivative thereof wherein V is N, Y is CR¹ wherein R¹ is as defined above in which Ar is unsubstituted furan or thiazole; U is indazole; R³ is benzyl or fluorobenzyl; and R⁴ is not present.

In another embodiment, the compound of formula (I) is a compound of formula (II): 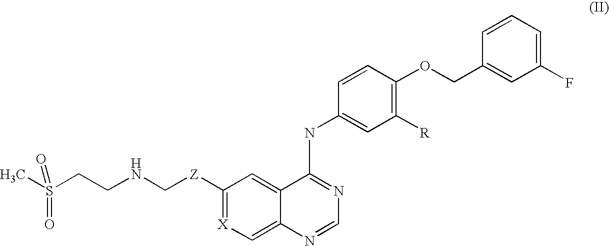
and salt or solvate thereof, wherein R is —Cl or —Br, X is CH, N, or CF, and Z is thiazole or furan.

In another embodiment, the compound of formula (I) is a compound of formula (III): 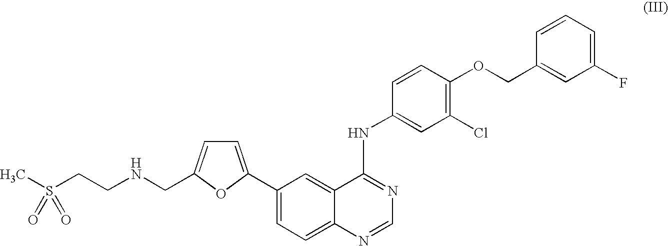
and salts or solvates thereof.

In another embodiment, the compound of formula (I) is a ditosylate salt of the compound of formula (III) and anhydrate or hydrate forms thereof. The ditosylate salt of the compound of formula (III) has the chemical name N-{3-Chloro-4-[(3-fluorobenzyl)oxy]phenyl}-6-[5-({[2-(methanesulphonyl)ethyl]amino}methyl)-2-furyl]-4-quinazolinamine ditosylate. In one embodiment, the compound of formula (I) is the anhydrous ditosylate salt of the compound of formula (III). In another embodiment, the compound of formula (I) is the monohydrate ditosylate salt of the compound of formula (III).

In another embodiment, the compound of formula (I) is a compound of formula (II) wherein, R is Cl; X is CH; and Z is thiazole. In a preferred embodiment, the compound of formula (I) is a ditosylate salt of a compound of formula (II) wherein, R is Cl; X is CH; and Z is thiazole; and anhydrate or hydrate forms thereof. The chemical name of such compound of formula (II) is (4-(3-Fluoro-benzyloxy)-3-chlorophenyl)-(6-(2-((2-methanesulphonyl-ethylamino)-methyl)-thiazol-4-yl)quinazolin-4-yl)-amine and is a compound of formula (III′).

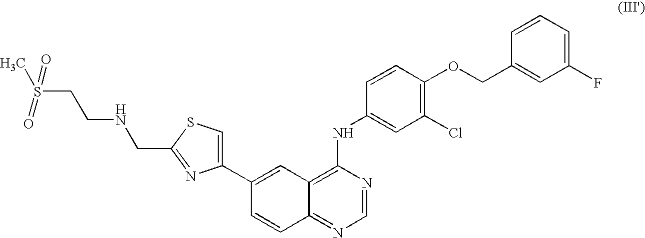

In another embodiment, the compound of formula (I) is a compound of formula (II) wherein, R is Br; X is CH; and Z is furan. In a preferred embodiment, the compound of formula (I) is a ditosylate salt of the compound of formula (II) wherein, R is Br; X is CH; and Z is furan; and anhydrate or hydrate forms thereof. The chemical name of such compound of formula (II) is (4-(3-Fluoro-benzyloxy)-3-bromophenyl)-(6-(5-((2-methanesulphonyl-ethylamino)-methyl)-furan-2-yl)quinazolin-4-yl)-amine and is a compound of formula (III″).

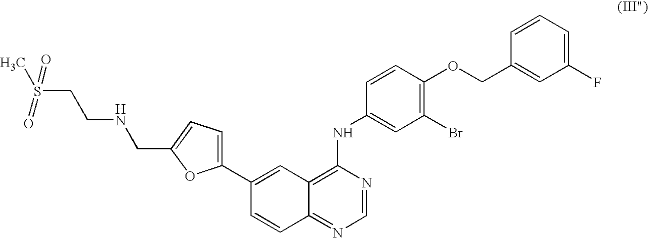

The free base, HCl salts, and ditosylate salts of the compounds of Formulae (I), (II), (III), (III′) and (III″) may be prepared according to the procedures of International Patent Application No. PCT/EP99100048, filed Jan. 8, 1999, and published as WO 99/35146 on Jul. 15, 1999, referred to above and International Patent Application No. PCT/US01/20706, filed Jun. 28, 2001 and published as WO 02/02552 on Jan. 10, 2002 and according the appropriate Examples recited below. One such procedure for preparing the ditosylate salt of the compound of formula (III) is presented following in Scheme 1.

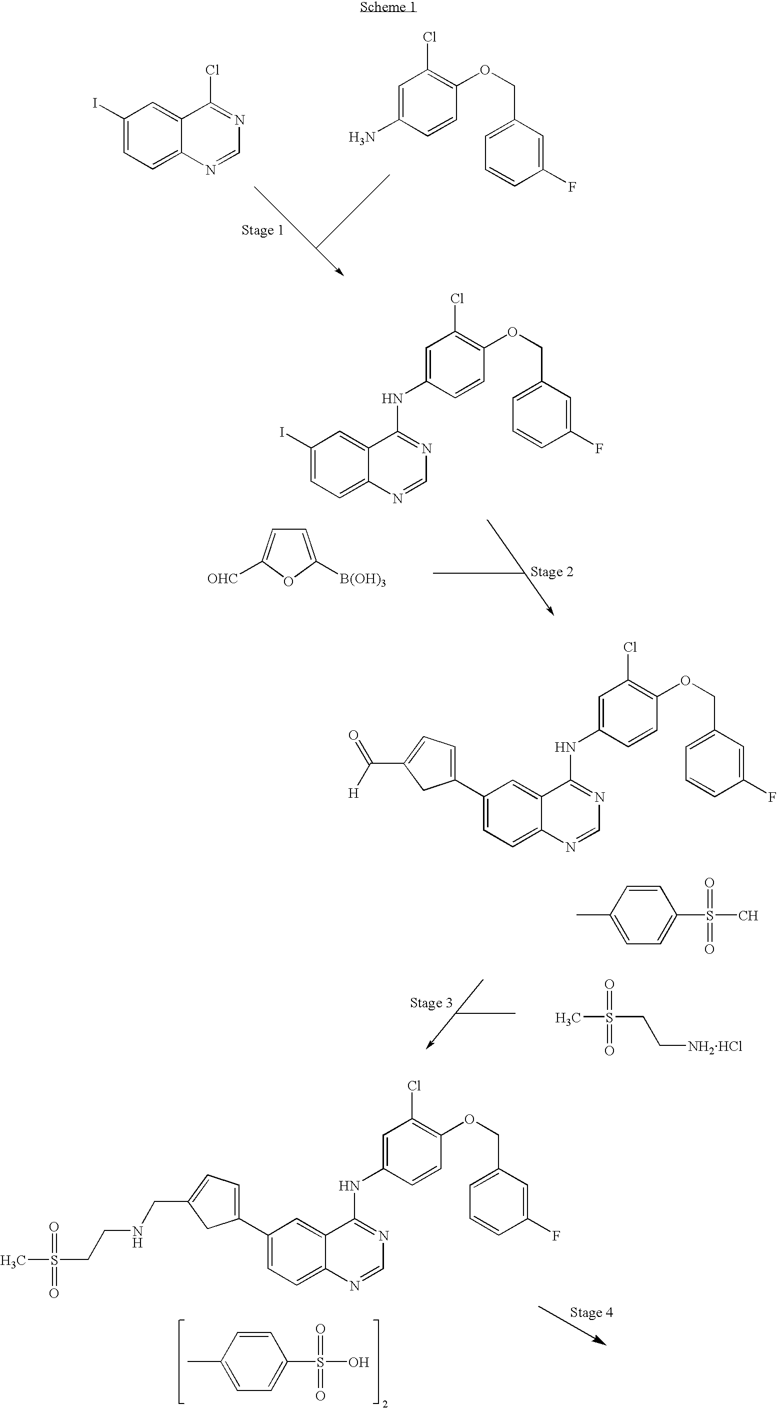

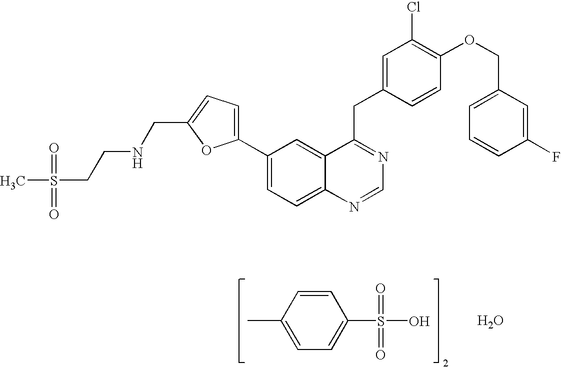

In scheme 1, the preparation of the ditosylate salt of the compound of formula (111) proceeds in four stages: Stage 1: Reaction of the indicated bicyclic compound and amine to give the indicated iodoquinazoline derivative; Stage 2: preparation of the corresponding aldehyde salt; Stage 3: preparation of the quinazoline ditosylate salt; and Stage 4: monohydrate ditosylate salt preparation.

Another class of dual EGFR/erbB-2 inhibitor compounds that may be usefully employed in the present invention includes compounds of the Formula I′: 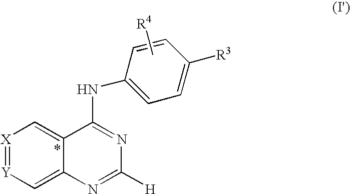
or a salt, solvate, or a physiologically functional derivative thereof;
wherein
X is CR¹ and Y is N;
or X is CR¹ and Y is CR²;
R¹ represents a group R⁵SO₂CH₂CH₂Z-(CH₂)ₚ—Ar—, wherein Ar is selected from phenyl, furan, thiophene, pyrrole and thiazole, each of which may optionally be substituted by one or two halo, C₁₋₄ alkyl or C₁₋₄ alkoxy groups; Z represents O, S, NH or NR⁶; p is 1, 2, 3 or 4;
R⁵ is C₁₋₆ alkyl optionally substituted by one or more R⁸ groups;
or R⁵ is C₁₋₆ alkyl substituted by a group Het or a group Cbc, each of which may be optionally substituted by one or more R⁸ groups;
or R⁵ is selected from a group Het or a group Cbc, each of which may be optionally substituted by one or more R⁸ groups;
each R⁸ is independently selected from halo, hydroxy, C₁₋₄ alkoxy, nitrile, NH₂ or NR⁶R⁷;
R⁶ is C₁₋₄ alkyl, C₁₋₄alkoxy-C₁₋₄alkyl, hydroxyC₁₋₄alkyl, CF₃C(O) or CH₃C(O);
R⁷ is hydrogen or R⁶;
R² is selected from hydrogen, halo, hydroxy, C₁₋₄ alkyl or C₁₋₄ alkoxy;
R³ is selected from pyridylmethoxy, benzyloxy, halo-, dihalo- or trihalobenzyloxy; and
R⁴ is selected from hydrogen, halogen, C₁₋₄ alkyl, C₂₋₄ alkynyl or cyano.

In a preferred embodiment, R⁴ is located on the phenyl ring as indicated in formula (I^{a}).

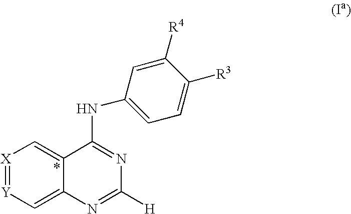

In one embodiment, the group R⁵ is an alkylene group linked to a Het or Cbc group, the alkylene group is preferably C₁₋₄alkylene, more preferably C₁₋₃ alkylene, most preferably methylene or ethylene.

The definitions for X and Y thus give rise to two possible basic ring systems for the compounds of formula (I′). In particular the compounds may contain the following basic ring systems: quinazolines (1) and pyrido-pyrimidines (2)

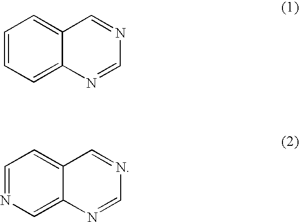

Ring system (1) is preferred.

The group Het comprise one or more rings which may be saturated, unsaturated, or aromatic and which may independently contain one or more nitrogen, oxygen, or sulfur heteroatoms, where N-oxides and sulfur monoxides and sulfur dioxides are permissible heteroaromatic substitutions in each ring.

Examples of suitable Het groups include acridine, benzimidazole, benzofuran, benzothiophene, benzoxazole, benzthiazole, carbazole, cinnoline, dioxin, dioxane, dioxalane, dithiane, dithiazine, dithiazole, dithiolane, furan, imidazole, imidazoline, imidazolidine, indole, indoline, indolizine, indazole, isoindole, isoquinoline, isoxazole, isothiazole, morpholine, napthyridine, oxazole, oxadiazole, oxathiazole, oxathiazolidine, oxazine, oxadiazine, phenazine, phenothiazine, phenoxazine, phthalazine, piperazine, piperidine, pteridine, purine, pyran, pyrazine, pyrazole, pyrazoline, pyrazolidine, pyridazine, pyridine, pyrimidine, pyrrole, pyrrolidine, pyrroline, quinoline, quinoxaline, quinazoline, quinolizine, tetrahydrofuran, tetrazine, tetrazole, thiophene, thiadiazine, thiadiazole, thiatriazole, thiazine, thiazole, thiomorpholine, thianaphthalene, thiopyran, triazine, triazole, or trithiane.

Preferred Het groups are aromatic groups selected from furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, and indazole.

More preferred Het groups are aromatic groups selected from furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine.

Most preferred Het groups are aromatic groups selected from pyridine and imidazole, especially pyrid-2-yl and imidazol-2-yl.

Cbc groups comprise one or more rings which may be independently saturated, unsaturated, or aromatic and which contain only carbon and hydrogen.

Preferred Cbc groups include aromatic groups selected from phenyl, biphenyl, naphthyl (including 1-naphthyl and 2-naphthyl) and indenyl.

Further suitable Cbc groups include cyclopropyl, cyclobutyl, cyclopentyl, cyclohexyl, cycloheptyl, cyclooctyl, tetralin, decalin, cyclopentenyl and cyclohexenyl.

A more preferred Cbc group is phenyl.

In one embodiment, Het groups and Cbc groups included within the group R⁵ are unsubstituted.

In a preferred embodiment, X is CR¹ and Y is CR² (ring system (1) above).

In a further preferred embodiment, X is CR¹ and Y is N (ring system (2) above.

In a preferred embodiment, R² represents hydrogen, halogen or C₁₋₄ alkoxy. In a more preferred embodiment R² represents hydrogen, fluoro or methoxy. In a most preferred embodiment R² represents hydrogen or fluoro.

In a preferred embodiment, Z represents NH, NR⁶ or O. In a more preferred embodiment Z presents NH or O. In a most preferred embodiment Z represents NH.

In a preferred embodiment, p is 1, 2 or 3.

In a further preferred embodiment, the group Ar does not carry any optional substituents.

In a further preferred embodiment, Ar represents furan or thiazole.

In a preferred embodiment, R⁵ represents an aromatic Het or Cbc group optionally substituted by a C₁₋₄alkyl group (especially a methyl group).

In a more preferred embodiment, R⁵ represents pyridyl (especially pyrid-2-yl), phenyl, imidazolyl or N-methylimidazolyl (especially imidazol-2-yl).

In a preferred embodiment, R⁵ represents C₁₋₆ alkyl optionally substituted by one or more groups selected from halo, hydroxy, C₁₋₄ alkoxy, nitrile, NH₂ or NR⁶R⁷, wherein R⁷ represents H or R⁶, wherein R⁶ is as defined above.

In a more preferred embodiment, R⁵ represents C₁₋₆ alkyl optionally substituted by one or more groups selected from hydroxy, C₁₋₄ alkoxy, NH₂ or NR⁶R⁷, wherein R⁷ represents H or R⁶; and R⁶ represents C₁₋₄ alkyl.

In a most preferred embodiment, R⁵ represents unsubstituted C₁₋₆ alkyl; especially unsubstituted C₁₋₄ alkyl.

The side chain R⁵SO₂CH₂CH₂Z-(CH₂)ₚ may be linked to any suitable position of the group Ar. Similarly, the group R¹ may be linked to the carbon atom carrying it from any suitable position of the group Ar.

In a more preferred embodiment, when Ar represents furan the side chain R⁵SO₂CH₂CH₂Z-(CH₂)ₚ is in the 5-position of the furan ring and the link to the carbon atom carrying the group R¹ is from the 2-position of the furan ring.

In a further more preferred embodiment, when Ar represents thiazole the side chain R⁵SO₂CH₂CH₂Z-(CH₂)ₚ is in the 2-position of the thiazole ring and the link to the carbon atom carrying the group R¹ is from the 4-position of the thiazole ring.

In a preferred embodiment, R³ represents benzyloxy or fluorobenzyloxy (especially 3-fluorobenzyloxy).

In an especially preferred embodiment, R⁴ represents chloro, bromo, or hydrogen.

In a most especially preferred embodiment, R³ is represents benzyloxy or 3-fluorobenzyloxy and R⁴ chloro or bromo.

In a more preferred embodiment, there is provided a compound of formula (I′) or a salt, solvate or physiologically functional derivative thereof wherein Y is CR², wherein R² is hydrogen, fluoro or methoxy; X is CR¹ wherein R¹ is as defined above in which Ar is unsubstituted furan or thiazole; R³ is benzyloxy or fluorobenzyloxy; and R⁴ is hydrogen, or is chloro or bromo.

In a further more preferred embodiment, there is provided a compound of formula (I′) or a salt or solvate thereof wherein Y is N; X is CR¹ wherein R¹ is as defined above in which Ar is unsubstituted furan or thiazole; R³ is benzyloxy or fluorobenzyloxy; and R⁴ is hydrogen, or is chloro or bromo.

In a most preferred embodiment, there is provided a compound of formula (I′) or a salt or solvate thereof wherein Y is CR², wherein R² is hydrogen, fluoro or methoxy; X is CR¹ wherein R¹ is as defined above in which Ar is unsubstituted furan or thiazole; R³ is fluorobenzyloxy; and R⁴ is chloro or bromo.

In a further most preferred embodiment, there is provided a compound of formula (I′) or a salt or solvate thereof wherein Y is N; X is CR¹ wherein R¹ is as defined above in which Ar is unsubstituted furan or thiazole; R³ is fluorobenzyloxy; and R⁴ is chloro or bromo.

In a more preferred embodiment, there is provided a compound of formula (I′) or a salt or solvate thereof wherein Y is CR², wherein R² is hydrogen, fluoro or methoxy; X is CR¹ wherein R¹ is as defined above in which Ar is unsubstituted furan or thiazole; R³ is benzyloxy or fluorobenzyloxy; R⁴ is hydrogen, or is chloro or bromo; and R⁵ is unsubstituted C₁₋₆ alkyl.

In a further more preferred embodiment, there is provided a compound of formula (I′) or a salt, solvate or physiologically functional derivative thereof wherein Y is N; X is CR¹ wherein R¹ is as defined above in which Ar is unsubstituted furan or thiazole; R³ is benzyloxy or fluorobenzyloxy; R⁴ is hydrogen, or is chloro or bromo; and R⁵ is unsubstituted C₁₋₆ alkyl.

In a most preferred embodiment, there is provided a compound of formula (I′) or a salt or solvate thereof wherein Y is CR², wherein R² is hydrogen, fluoro or methoxy; X is CR¹ wherein R¹ is as defined above in which Ar is unsubstituted furan or thiazole; R³ is fluorobenzyloxy; R⁴ is chloro or bromo; and R⁵ is unsubstituted C₁₋₆ alkyl.

In a further most preferred embodiment, there is provided a compound of formula (I′) or a salt or solvate thereof wherein Y is N; X is CR¹ wherein R¹ is as defined above in which Ar is unsubstituted furan or thiazole; R³ is fluorobenzyloxy; R⁴ is chloro or bromo; and R⁵ is unsubstituted C₁₋₆ alkyl.

In a more preferred embodiment, there is provided a compound of formula (I′) or a salt or solvate thereof wherein Y is CR², wherein R² is hydrogen, fluoro or methoxy; X is CR¹ wherein R¹ is as defined above in which Ar is unsubstituted furan or thiazole; R³ is benzyloxy or fluorobenzyloxy; R⁴ is hydrogen, or is chloro or bromo; and
R¹ is pyridine, imidazole, or phenyl.

In a further more preferred embodiment, there is provided a compound of formula (I′) or a salt, solvate or physiologically functional derivative thereof wherein Y is N; X is CR¹ wherein R¹ is as defined above in which Ar is unsubstituted furan or thiazole; R³ is benzyloxy or fluorobenzyloxy; R⁴ is hydrogen, or is chloro or bromo; and
R¹ is pyridine, imidazole, or phenyl.

In a most preferred embodiment, there is provided a compound of formula (I′) or a salt or solvate thereof wherein Y is CR², wherein R² is hydrogen, fluoro or methoxy; X is CR¹ wherein R¹ is as defined above in which Ar is unsubstituted furan or thiazole; R³ is fluorobenzyloxy; R⁴ is chloro or bromo; and R⁵ is pyridine, imidazole, or phenyl.

In a further most preferred embodiment there is provided a compound of formula (I′) or a salt or solvate thereof wherein Y is N; X is CR¹ wherein R¹ is as defined above in which Ar is unsubstituted furan or thiazole; R³ is fluorobenzyloxy; R⁴ is chloro or bromo; and R⁵ is pyridine, imidazole, or phenyl.

A group of preferred species of compounds of Formula (I′) are:

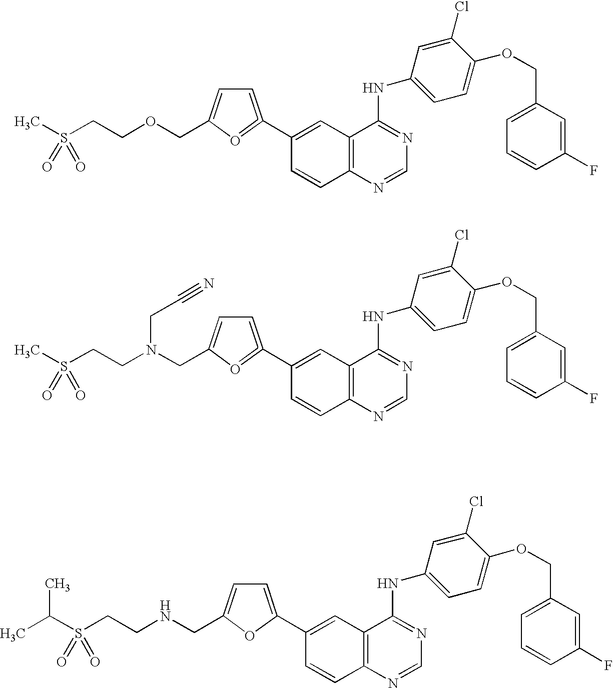

The compounds of Formulae (I′) and (1^{a}) may be prepared according to the procedures of International Patent Application No. PCT/US00/18128, filed Jun. 30, 2000, and published as WO 01/04111 on Jan. 18, 2001, referred to above and according to the appropriate Examples recited below.

Still another class of dual EGFR/erbB-2 inhibitor compounds that may be usefully employed in the present invention includes compounds of the Formula I″: 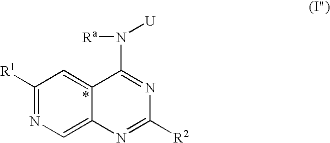
or a salt, solvate, or physiologically functional derivative thereof;
wherein
R^{a} is hydrogen or a C₁₋₈ alkyl group
R¹ is independently selected from the group comprising amino, hydrogen, halo, hydroxy, nitro, carboxy, formyl, cyano, trifluoromethyl, trifluoromethoxy, carbamoyl, ureido, guanidino, C₁₋₈ alkyl, C₁₋₈ alkoxy, C₃₋₈ cycloalkoxy, C₄₋₈ alkylcycloalkoxy, C₁₋₈ alkylcarbonyl, C₁₋₈ alkoxycarbonyl, N—C₁₋₄ alkylcarbamoyl, N,N-di-[C₁₋₄ alkyl]carbamoyl, hydroxyamino, C₁₋₄ alkoxyamino, C₂₋₄ alkanoyloxyamino, C₁₋₄ alkylamino, di[C₁₋₄ alkyl]amino, di-[C₁₋₄ alkyl]amino-C₁₋₄ alkylene-(C₁₋₄ alkyl)amino, C₁₋₄ alkylamino-C₁₋₄ alkylene-(C₁₋₄ alkyl)amino, hydroxy-C₁₋₄ alkylene-(C₁₋₄ alkyl)amino, phenyl, phenoxy, 4-pyridon-1-yl, pyrrolidin-1-yl, imidazol-1-yl, piperidino, morpholino, thiomorpholino, thiomorpholino-1-oxide, thiomorpholino-1,1-dioxide, piperazin-1-yl, 4-C₁₋₄ alkylpiperazin-1-yl, dioxolanyl, C₁₋₈ alkylthio, arylthio, C₁₋₄ alkylsulphinyl, C₁₋₄ alkylsulphonyl, arylsulphinyl, arylsulphonyl, halogeno-C₁₋₄ alkyl, hydroxy-C₁₋₄ alkyl, C₂₋₄ alkanoyloxy-C₁₋₄ alkyl, C₁₋₄ alkoxy-C₁₋₄ alkyl, carboxy-C₁₋₄ alkyl, formyl-C₁₋₄ alkyl, C₁₋₄ alkoxycarbonyl-C₁₋₄-alkyl, carbamoyl-C₁₋₄ alkyl, N-C₁₋₄ alkylcarbamoyl-C₁₋₄alkyl, N,N-di-[C₁₋₄ alkyl]carbamoyl-C₁₋₄alkyl, amino-C₁₋₄ alkyl, C₁₋₄ alkylamino-C₁₋₄ alkyl, di-[C₁₋₄ alkyl]amino-C₁₋₄ alkyl, phenyl-C₁₋₄ alkyl, 4-pyridon-1-yl-C₁₋₄ alkyl, pyrrolidin-1-yl-C₁₋₄ alkyl, imidazol-1-yl-C₁₋₄ alkyl, piperidino-C₁₋₄ alkyl, morpholino-C₁₋₄ alkyl, thiomorpholino-C₁₋₄alkyl, thiomorpholino-1-oxide-C₁₋₄alkyl, thiomorpholino-1,1-dioxide-C₁₋₄alkyl, piperazin-1-yl-C₁₋₄alkyl, 4-C₁₋₄ alkylpiperazin-1-yl-C₁₋₄ alkyl, hydroxy-C₂₋₄ alkoxy-C₁₋₄ alkyl, C₁₋₄ alkoxy-C₂₋₄ alkoxy-C₁₋₄ alkyl, hydroxy-C₂₋₄ alkylamino-C₁₋₄ alkyl, C₁₋₄ alkoxy-C₂₋₄ alkylamino-C₁₋₄ alkyl, C₁₋₄ alkylthio-C₁₋₄ alkyl, hydroxy-C₂₋₄ alkylthio-C₁₋₄ alkyl, C₁₋₄ alkoxy-C₂₋₄ alkylthio-C₁₋₄ alkyl, phenoxy-C₁₋₄ alkyl, anilino-C₁₋₄ alkyl, phenylthio-C₁₋₄ alkyl, cyano-C₁₋₄ alkyl, halogeno-C₂₋₄ alkoxy, hydroxy-C₂₋₄ alkoxy, C₂₋₄ alkanoyloxy-C₂₋₄ alkoxy, C₁₋₄ alkoxy-C₂₋₄ alkoxy, carboxy-C₁₋₄ alkoxy, formyl-C₁₋₄ alkoxy, C₁₋₄ alkoxycarbonyl-C₁₋₄ alkoxy, carbamoyl-C₁₋₄ alkoxy, N—C₁₋₄ alkylcarbamoyl-C₁₋₄ alkoxy, N,N-di-[C₁₋₄ alkyl]carbamoyl-C₁₋₄ alkoxy, amino-C₂₋₄ alkoxy, C₁₋₄ alkylamino-C₂₋₄ alkoxy, di-[C₁₋₄ alkyl]amino-C₂₋₄ alkoxy, di-[C₁₋₄ alkyl-C₂₋₄ alkoxy]amino-C₂₋₄ alkoxy, C₂₋₄ alkanoyloxy, hydroxy-C₂₋₄ alkanoyloxy, C₁₋₄alkoxy-C₂₋₄ alkanoyloxy, phenyl-C₁₋₄ alkoxy, phenoxy-C₂₋₄ alkoxy, anilino-C₂₋₄ alkoxy, phenylthio-C₂₋₄ alkoxy, 4-pyridon-1-yl-C₂₋₄ alkoxy, piperidino-C₂₋₄ alkoxy, pyrrolidin-1-yl-C₂₋₄ alkoxy, imidazol-1-yl-C₂₋₄ alkoxy, morpholino-C₂₋₄ alkoxy, thiomorpholino-C₂₋₄ alkoxy, thiomorpholino-1-oxide-C₂₋₄ alkoxy, thiomorpholino-1,1-dioxide-C₂₋₄ alkoxy, piperazin-1-yl-C₂₋₄ alkoxy, 4-C₁₋₄ alkylpiperazin-1-yl-C₂₋₄ alkoxy, halogeno-C₂₋₄ alkylamino, hydroxy-C₂₋₄ alkylamino, C₂₋₄ alkanoyloxy-C₂₋₄ alkylamino, C₁₋₄ alkoxy-C₂₋₄ alkylamino, carboxy-C₁₋₄ alkylamino, C₁₋₄ alkoxycarbonyl-C₁₋₄alkylamino, carbamoyl-C₁₋₄alkylamino, N—C₁₋₄ alkylcarbamoyl-C₁₋₄ alkylamino, N,N-di-[C₁₋₄ alkyl]carbamoyl-C₁₋₄ alkylamino, amino-C₁₋₄ alkylamino, C₁₋₄ alkylamino-C₂₋₄ alkylamino, di-[C₁₋₄ alkyl]amino-C₂₋₄ alkylamino, phenyl-C₁₋₄ alkylamino, phenoxy-C₂₋₄ alkylamino, anilino-C₂₋₄ alkylamino, 4-pyridon-1-yl-C₂₋₄ alkylamino, pyrrolidin-1-yl-C₂₋₄ alkylamino, imidazol-1-yl-C₂₋₄ alkylamino, piperidino-C₂₋₄ alkylamino, morpholino-C₂₋₄ alkylamino, thiomorpholino-C₂₋₄ alkylamino, thiomorpholino-1-oxide-C₂₋₄ alkylamino, thiomorpholino-1,1-dioxide-C₂₋₄ alkylamino, piperazin-1-yl-C₂₋₄ alkylamino, 4-(C₁₋₄ alkyl)piperazin-1-yl-C₂₋₄ alkylamino, phenylthio-C₂₋₄ alkylamino, C₂₋₄ alkanoylamino, C₁₋₄ alkoxycarbonylamino, C₁₋₄ alkylsulphonylamino, C₁₋₄ alkylsulphinylamino, benzamido, benzenesulphonamido, 3-phenylureido, 2-oxopyrrolidin-1-yl, 2,5-dioxopyrrolidin-1-yl, halogeno-C₂₋₄ alkanoylamino, hydroxy-C₂₋₄ alkanoylamino, hydroxy-C₂₋₄ alkanoyl-(C₁₋₄ alkyl)-amino, C₁₋₄ alkoxy-C₂₋₄ alkanoylamino, carboxy-C₂₋₄ alkanoylamino, C₁₋₄ alkoxycarbonyl-C₂₋₄ alkanoylamino, carbamoyl-C₂₋₄ alkanoylamino, N—C₁₋₄ alkylcarbamoyl-C₂₋₄ alkanoylamino, N,N-di-[C₁₋₄ alkyl]carbamoyl-C₂₋₄ alkanoylamino, amino-C₂₋₄ alkanoylamino, C₁₋₄ alkylamino-C₂₋₄ alkanoylamino or di-[C₁₋₄ alkyl]amino-C₂₋₄ alkanoylamino, and wherein said benzamido or benzenesulphonamido substituent or any anilino, phenoxy or phenyl group on a R¹ substituent may optionally bear one or two halo, C₁₋₄ alkyl or C₁₋₄ alkoxy substituents; and wherein any substituent containing a Het ring may optionally bear one or two halo, C₁₋₄alkyl or C₁₋₄alkoxy substituents on said ring; and wherein any substituent containing a Het ring may optionally bear one or two oxo or thioxo substituents on said ring;
or R¹ represents a group selected from M¹-M²-M³-M⁴, M¹-M⁵ or M¹-M²-M^{3′}-M⁶ wherein
M¹ represents a C₁₋₄ alkyl group, wherein optionally a CH₂ group is replaced by a CO group;
M² represents NR¹² or CR¹²R¹³, in which R¹² and R¹³ each independently represent H or C₁₋₄ alkyl;
M³ represents a C₁₋₄ alkyl group;
M³ represents a C₁₋₄ alkyl group or is absent;
M⁴ represents CN, NR¹²S(O)ₘR¹³, S(O)ₘNR¹⁴R¹⁵, CONR¹⁴R¹⁵, S(O)ₘR¹³ or CO₂R¹³, in which R¹², R¹³ and m are as hereinbefore defined and R¹⁴ and R¹⁵ each independently represent H or C₁₋₄ alkyl, or R¹⁴ and R¹⁵ together with the nitrogen atom to which they are attached represent a 5- or 6-membered ring optionally containing 1 or 2 additional heteroatoms selected from N, O or S(O)ₘ in which ring any nitrogen atom present may optionally be substituted with a C₁₋₄ alkyl group, and which ring may optionally bear one or two oxo or thioxo substituents;
M⁵ represents the group NR¹⁴R¹⁵, wherein R¹⁴ and R¹⁵ are as defined above, or M⁵ represents the group 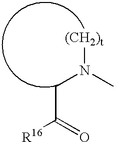
   in which t represents 2 to 4 and R¹⁶ represents OH, OC₁₋₄ alkyl or NR¹⁴R¹⁵; and
M⁶ represents a C₃₋₆ cycloalkyl group, the group NR¹⁴R¹⁵, wherein R¹⁴ and R¹⁵ are as defined above, or a 5- or 6-membered Het ring system containing 1 to 4 heteroatoms selected from N, O or S;
and p is 0 to 3; or when p is 2 or 3, two adjacent R¹ groups together form an optionally substituted methylenedioxy or ethylenedioxy group;
R² is selected from the group comprising hydrogen, halogen, trifluoromethyl, C₁₋₄ alkyl and C₁₋₄ alkoxy;
U represents a 5 to 10-membered mono or bicyclic ring system in which one or more of the carbon atoms is optionally replaced by a heteroatom independently selected from N, O and S(O)ₘ, wherein m is 0, 1 or 2 and wherein the ring system is substituted by at least one independently selected R⁶ group and is optionally substituted by at least one independently selected R⁴ group;
each R⁴ is independently hydrogen, hydroxy, halogen, C₁₋₄ alkyl, C₁₋₄ alkoxy, C₁₋₄ alkylamino, di-[C₁₋₄ alkyl]amino, C₁₋₄ alkylthio, C₁₋₄ alkylsulphinyl, C₁₋₄ alkylsulphonyl, C₁₋₄ alkylcarbonyl, C₁₋₄ alkylcarbamoyl, di-[C₁₋₄ alkyl]carbamoyl, carbamyl, C₁₋₄ alkoxycarbonyl, cyano, nitro or trifluoromethyl;
each R⁶ is independently a group ZR⁷ wherein Z is joined to R⁷ through a (CH₂)ₚ group in which p is 0, 1 or 2 and Z represents a group V(CH₂), V(CF₂), (CH₂)V, (CF₂)V, V(CRR′), V(CHR) or V where R and R¹ are each C₁₋₄ alkyl and in which V is a hydrocarbyl group containing 0, 1 or 2 carbon atoms, carbonyl, dicarbonyl, CH(OH), CH(CN), sulphonamide, amide, O, S(O)ₘ or NR^{b} where R^{b} is hydrogen or R^{b} is C₁₋₄ alkyl; and R⁷ is an optionally substituted C₃₋₆ cycloalkyl; or an optionally substituted 5, 6, 7, 8, 9 or 10-membered Cbc or Het moiety;
or R⁶ is a group ZR⁷ in which Z is NR^{b}, and NR^{b} and R⁷ together form an optionally substituted 5, 6, 7, 8, 9 or 10-membered carbocyclic or heterocyclic moiety.

Het groups comprise one or more rings which may be saturated, unsaturated, or aromatic and which may independently contain one or more heteroatoms in each ring.

Cbc groups comprise one or more rings which may be independently saturated, unsaturated, or aromatic and which contain only carbon and hydrogen.

Suitably the 5, 6, 7, 8, 9 or 10-membered Het moiety is selected from the group comprising: furan, dioxolane, thiophene, pyrrole, imidazole, pyrrolidine, pyran, pyridine, pyrimidine, morpholine, piperidine, oxazole, isoxazole, oxazoline, oxazolidine, thiazole, isothiazole, thiadiazole, benzofuran, indole, isoindole, quinazoline, quinoline, isoquinoline and ketal.

Suitably the 5, 6, 7, 8, 9 or 10-membered Cbc moiety is selected from the group comprising: phenyl, benzyl, indene, naphthalene, tetralin, decalin, cyclopentyl, cyclopentenyl, cyclohexyl, cyclohexenyl and cycloheptyl.

In an embodiment, R¹ is as defined above with the exception of wherein any substituent containing a Het ring bears one or two oxo or thioxo substituents on said ring; and R¹⁴ and R¹⁵ are as defined above with the exception of wherein they together with the nitrogen atom to which they are attached represent a 5- or 6-membered ring and said ring bears one or two oxo or thioxo substituents; save that R¹ may represent 4-pyridon-1-yl, 4-pyridon-1-yl-C₁₋₄ alkyl, 4-pyridon-1-yl-C₂₋₄ alkoxy, 4-pyridon-1-yl-C₂₋₄ alkylamino, 2-oxopyrrolidin-1-yl or 2,5-dioxopyrrolidin-1-yl.

In a further embodiment, R¹ is selected from the group comprising amino, hydrogen, halogen, hydroxy, formyl, carboxy, cyano, nitro, C₁₋₈ alkyl, C₁₋₈ alkoxy, C₁₋₈ alkylthio, C₁₋₈ alkylsulphinyl, C₁₋₈ alkylsulphonyl, C₁₋₄ alkylamino, C₁₋₄ dialkylamino, dioxolanyl, benzyloxy or hydroxy-C₁₋₄ alkanoyl-(C₁₋₄ alkyl)-amino.

In a preferred embodiment, R¹ is selected from the group comprising amino, C₁₋₄ alkylamino, diC₁₋₄ alkylamino, especially diC₁₋₄ alkylamino, most especially dimethylamino or methylethylamino.

In a further embodiment, R¹ is selected from M¹-M²-M³-M⁴, M¹-M⁵ or M¹-M²-M^{3′}-M⁶ as defined above; and p=1.

In a further embodiment, the group M²-M³-M⁴ represents an α-, β- or γ-amino carboxylic, sulphinic or sulphonic acid or a C₁₋₄ alkyl ester, an amide or a C₁₋₄alkyl- or di-(C₁₋₄ alkyl)-amide thereof.

Preferably M¹ represents CH₂, CO, CH₂CH₂ or CH₂CO, more preferably CH₂.

Preferably M² represents NR¹² in which R¹² is as defined above; more preferably R¹² represents H or methyl.

Preferably M³ represents CH₂, CH₂CH₂ or propyl.

Preferably M^{3′} represents CH₂, ethyl, propyl, isopropyl or is absent.

Preferably M⁴ represents SOR¹³, SO₂R¹³, NR¹²SO₂R¹³, CO₂R¹³ or CONR¹⁴R¹⁵ in which R¹² and R¹³ are defined above and R¹⁴ and R¹⁵ each independently represent H or C₁₋₄ alkyl; more preferably R¹², R¹³, R¹⁴ and R¹⁵ each independently represent H or methyl.

Preferably M⁵ represents a group NR¹⁴R¹⁵ in which R¹⁴ and R¹⁵ together with the nitrogen atom to which they are attached represent a 6-membered ring optionally containing an additional heteroatom selected from N or O, in which ring any nitrogen atom present may optionally be substituted with a C₁₋₄ alkyl group, preferably a methyl group; or M⁵ represents a group 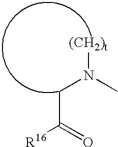
in which t represents 2 or 3 and R¹⁶ represents OH, NH₂, N(C₁₋₄ alkyl)₂ or OC₁₋₄ alkyl; more preferably R¹⁶ represents NH₂ or N(CH₃)₂.

M⁵ also preferably represents a group NR¹⁴R¹⁵ in which R¹⁴ and R¹⁵ each independently represent hydrogen or C₁₋₄ alkyl, more preferably hydrogen, methyl, ethyl or isopropyl.

Preferably M⁶ represents a group NR¹⁴R¹⁵ in which R¹⁴ and R¹⁵ each independently represent C₁₋₄alkyl, more preferably methyl, or R¹⁴ and R¹⁵ together with the nitrogen atom to which they are attached represent a 5- or 6-membered ring optionally containing an additional heteroatom selected from N or O, in which ring any nitrogen atom present may optionally be substituted with a C₁₋₄ alkyl group, preferably a methyl group; or M⁶ represents a 5- or 6-membered Het ring system containing 1 or 2 heteroatoms selected from N or O.

In a further preferred embodiment, M²-M³-M⁴ represents an α-amino carboxylic acid or a methyl ester or amide thereof.

In a further preferred embodiment, M²-M³-M⁴ represents an α-, β- or γ-amino sulphinic or sulphonic acid, more preferably a β- or γ-amino sulphinic or sulphonic acid, most preferably a β-aminosulphonic acid, or a methyl ester thereof.

In an especially preferred embodiment, M²-M³-M⁴ represents a methylsulphonylethylamino, methylsulphinylethylamino, methylsulphonylpropylamino, methylsulphinylpropylamino, methylsulphonamidoethylamino, sarcosinamide, glycine, glycinamide, glycine methyl ester or acetylaminoethylamino group.

In a further especially preferred embodiment, M⁵ represents a piperazinyl, methylpiperazinyl, piperidinyl, prolinamido or N,N-dimethylprolinamido group.

In a further especially preferred embodiment, M⁵ represents an isopropylamino or N-morpholinyl group.

In a further especially preferred embodiment, M¹-M⁵ represents an isopropylacetamido or N-morpholinoacetamido group.

In a further especially preferred embodiment, M²-M^{3′}-M⁶ represents a pyridylamino, cyclopropylamino, N-(piperidin-4-yl)-N-methylamino, N,N-dimethylaminoprop-2-ylamino, N-(2-dimethylaminoethyl)-N-ethylamino or tetrahydrofuranomethylamino group, preferably a pyridylamino group.

In a further embodiment, each R¹ is independently selected from the group comprising amino, hydrogen, halogen, hydroxy, formyl, carboxy, cyano, nitro, C₁₋₈ alkyl, C₁₋₈ alkoxy, C₁₋₈ alkylthio, C₁₋₈ alkylsulphinyl, C₁₋₈ alkylsulphonyl, C₁₋₄ alkylamino, C₁₋₄ dialkylamino, benzyloxy, hydroxy-C₁₋₄ alkyl, hydroxy-C₁₋₄ alkanoyl-(C₁₋₄ alkyl)-amino.

In an embodiment, R² is hydrogen, C₁₋₄ alkyl, C₁₋₄ alkoxy or halogen, preferably methyl or hydrogen, more preferably hydrogen.

In a further embodiment, R⁴ is hydrogen, hydroxy, halogen, C₁₋₄ alkyl, C₁₋₄ alkoxy, di-[C₁₋₄ alkyl]amino, nitro or trifluoromethyl, preferably hydrogen, halogen or methyl, more preferably hydrogen.

In a preferred embodiment, R¹ is an optionally substituted phenyl, dioxolanyl, thienyl, cyclohexyl or pyridyl group.

In a further embodiment, Z is absent or represents oxygen, CH₂, NR^{b}, NR^{b}(CH₂), (CH₂)NR^{b}, CH(CH₃), O(CH₂), (CH)CN, O(CF₂), (CH₂)O, (CF₂)O, S(CH₂), S(O)ₘ, carbonyl or dicarbonyl, wherein R^{b} is hydrogen or C₁₋₄ alkyl.

In a preferred embodiment, Z is oxygen, dicarbonyl, OCH₂, CH₂(CN), S(O)ₘ or NR^{b}, wherein R^{b} is hydrogen or C₁₋₄ alkyl.

In a further preferred embodiment, R⁶ is benzyl, halo-, dihalo- and trihalobenzyl, α-methylbenzyl, phenyl, halo-, dihalo- and trihalophenyl, pyridyl, pyridylmethyl, pyridyloxy, pyridylmethoxy, thienylmethoxy, dioxolanylmethoxy, cyclohexylmethoxy, phenoxy, halo-, dihalo- and trihalophenoxy, phenylthio, benzyloxy, halo-, dihalo- and trihalobenzyloxy, C₁₋₄ alkoxybenzyloxy, phenyloxalyl or benzenesulphonyl, more preferably benzyl, fluorobenzyl, benzyloxy, fluorobenzyloxy, pyridylmethyl, phenyl, benzenesulphonyl, phenoxy or fluorophenoxy.

In a further embodiment, R⁶ is in the para position with respect to the aniline N.

When the group Z is absent, R⁶═R⁷.

One or both of the rings comprising the mono or bicyclic ring system U may be aromatic or non-aromatic. The R⁴ and R⁶ groups may be bound to the ring system by either a carbon atom or a heteroatom of the ring system. The ring system itself may be bound to the bridging group by a carbon atom or a heteroatom. The R⁴ and R⁶ groups may be bound to either ring when U represents a bicyclic ring system, but these groups are preferably bound to the ring, which is not bound to the bridging group Y in such a case.

Examples of suitable mono or bicyclic groups U include: isoindenyl, indenyl, indanyl, naphthyl, 1,2-dihydronaphthyl or 1,2,3,4-tetrahydronaphthyl, pyrrolyl, pyridinyl, pyridazinyl, pyrimidinyl, pyrazinyl, furanyl, 2H-pyranyl, thiophenyl, 1H-azepinyl, oxepinyl, thiepinyl, azocinyl, 2H-oxocinyl, thieno[2,3-b]furanyl, thianaphthenyl, indolyl, indolinyl, isoindolyl, isoindolinyl, indolizinyl, 1H-benzimidazolyl, 2,3-dihydro-1H-benzimidazolyl, 1H-indazolyl, 2,3-dihydro-1H-indazolyl, benzoxazolyl, 2,3-dihydrobenzoxazolyl, benzo[c]isoxazolyl, benzo[d]isoxazolyl, 2,3-dihydrobenzo[d]isoxazolyl, benzothiazoyl, 2,3-dihydrobenzothiazolyl, benzo[c]isothiazolyl, benzo[d]isothiazolyl, 2,3-dihydrobenzo[d]isothiazolyl, 1H-benzotriazolyl, benzo[c]furanyl, benzo[c][1,2,3]thiadiazolyl, benzo[d][1,2,3]oxadiazolyl, benzo[d][1,2,3]thiadiazolyl, quinolyl, 1,2-dihydroquinolinyl, 1,2,3,4-tetrahydroquinolinyl, isoquinolyl 1,2,3,4-tetrahydroisoquinolinyl, cinnolinyl, quinazolinyl, quinoxalinyl, phthalazinyl, 4H-1,4-benzoxazinyl, 2,3-dihydro-4H-1,4-benzoxazinyl, 4H-1,4-benzothiazinyl or 2,3-dihydro-4H-1,4-benzothiazinyl.

Suitably U represents an indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group.

In an embodiment, the optional substitutents for the Cbc or Het moiety, which may be present at any available position of said moiety, are selected from the group comprising:
(CH₂)_{q}S(O)ₘ—C₁₋₄alkyl, (CH₂)_{q}S(O)ₘ—C₃₋₆cycloalkyl, (CH₂)_{q}SO₂NR⁸R⁹, (CH₂)_{q}NR⁸R⁹, (CH₂)_{q}CO₂R⁸, (CH₂)_{q}OR⁸, (CH₂)_{q}CONR⁸R⁹, (CH₂)_{q}NR⁸COR⁹, (CH₂)_{q}COR⁸, (CH₂)_{q}R⁸, NR⁸SO₂R⁹ and S(O)ₘR⁸,
wherein q is an integer from 0 to 4 inclusive; m is 0, 1 or 2;
R⁸ and R⁹ are independently selected from the group comprising hydrogen, C₁₋₄ alkyl, C₃₋₆ cycloalkyl, aryl, a 5- or 6-membered saturated or unsaturated Het ring which may be the same or different and which contains one or more heteroatoms which are selected from N, O or S(O)ₘ, with the proviso that the Het ring does not contain two adjacent O or S(O)ₘ atoms.

In a further embodiment, the optional substitutents for the Cbc or Het moiety are selected from the group comprising morpholine, piperazine, piperidine, pyrrolidine, tetrahydrofuran, dioxolane, oxothiolane and oxides thereof, dithiolane and oxides thereof, dioxane, pyridine, pyrimidine, pyrazine, pyridazine, furan, thiofuran, pyrrole, triazine, imidazole, triazole, tetrazole, pyrazole, oxazole, oxadiazole and thiadiazole.

Other optional substituents for the Cbc or Het moiety and also for other optionally substituted groups include, but are not limited to, hydroxy, halogen, trifluoromethyl, trifluoromethoxy, nitro, amino, cyano, C₁₋₄ alkoxy, C₁₋₄ alkylthio, C₁₋₄ alkyl carbonyl, carboxylate and C₁₋₄ alkoxy carboxyl.

In a further preferred embodiment, there is provided a compound of formula (I″) or a salt, solvate, or physiologically functional derivative thereof, wherein R^{a} is hydrogen or C₁₋₄ alkyl; R¹ group is selected from hydrogen, halo, C₁₋₄ alkyl, carboxy, formyl, hydroxy-C₁₋₄ alkyl, 1,3-dioxolan-2-yl, benzyloxy, amino, C₁₋₄alkylamino, di(C₁₋₄ alkyl)amino, hydroxy-C₁₋₄alkanoyl(C₁₋₄alkyl)amino, C₁₋₄alkylamino-C₁₋₄alkyl, di(C₁₋₄ alkyl)amino-C₁₋₄ alkyl, methylsulphonylethylaminomethyl, methylsulphonylethylamino-carbonyl, methylsulphinylethylamino-methyl, methylsulphinylethylamino-carbonyl, methylsulphonylpropylamino-methyl, methylsulphinylpropylamino-methyl, methylsulphonylpropyamino-carbonyl, methylsulphinylpropylamino-carbonyl, methylsulphonylethyl-(methylamino)-methyl, methylsulphonylethyl-(methylamino)-carbonyl, methylsulphinylethyl-(methylamino)-methyl, methylsulphinylethyl-(methylamino)-carbonyl, methylsulphonylpropyl-(methylamino)-methyl, methylsulphinylpropyl-(methylamino)-methyl, methylsulphonylpropyl-(methylamino)-carbonyl, methylsulphinyl propyl-(methylamino)-carbonyl, methylsulphonamidoethylamino-methyl, methylsulphonamidopropylamino-methyl, sarcosinamidomethyl, glycinylmethyl, glycinamidomethyl, glycinylmethyl methyl ester, acetylaminoethylaminomethyl, piperazinylmethyl, methylpiperazinylmethyl, piperidinylmethyl, N-(prolinamido)methyl, (N,N-dimethyl-prolinamido)methyl, pyridylaminomethyl, cyclopropylaminomethyl, N-(piperidin-4-yl)-N-methylaminomethyl, N,N-dimethylaminoprop-2-ylaminomethyl, N-(2-dimethylaminoethyl)-N-ethylaminomethyl, isopropylacetamido, N-morpholinylacetamido or tetrahydrofuranomethylaminomethyl; R² represents hydrogen; R⁴ represents hydrogen or methyl; U represents indolyl, benzimidazolyl or indazolyl, more preferably indazolyl; and R⁶ represents phenyl, benzyl, α-methylbenzyl, fluorobenzyl, benzenesulphonyl, phenoxy, fluorophenoxy, benzyloxy or fluorobenzyloxy.

In a further especially preferred embodiment, there is provided a compound of formula (I″) or a salt, solvate, or physiologically functional derivative thereof wherein R^{a} is hydrogen or C₁₋₄ alkyl; R¹ group is selected from hydrogen, halo, benzyloxy, amino, C₁₋₄ alkylamino, di(C₁₋₄ alkyl)amino or hydroxy-C₁₋₄ alkanoyl(C₁₋₄ alkyl)amino, more preferably dimethylamino; R² represents hydrogen; R⁴ represents hydrogen or methyl; U represents indazolyl, indolyl or benzimidazolyl, more preferably indazolyl; and R⁶ represents benzyl, fluorobenzyl, pyridylmethyl or benzenesulphonyl.

A preferred species of a compound of Formula (I″) is:

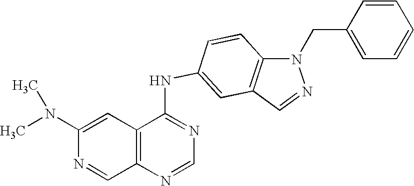

The compounds of Formula (I″) may be prepared according to the procedures of U.S. Pat. No. 6,174,889 and according to the appropriate Examples recited below.

As recited above the method and treatment combination of the present invention also includes a Raf family and/or a ras inhibitor. Preferably the Raf family inhibitor is a cRaf-1 inhibitor. Generally any cRaf-1 inhibitor, that is any pharmaceutical agent having specific cRaf-1 inhibitor activity may be utilized in the present invention. Such cRaf-1 inhibitors are described, for instance, in U.S. Pat. No. 6,268,391; and International Patent Applications WO 98/52559; WO 99/32106; WO 99/32455; WO 99/32436; and WO 00/42012 which patents and patent applications are herein incorporated by reference to the extent of their disclosure of cRaf-1 inhibitor compounds and methods of making and using the same.

One class of cRaf-1 inhibitor compounds that may be usefully employed in the present invention includes compounds of the Formula IV: 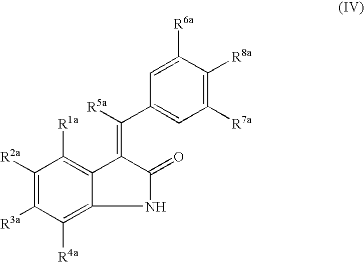
wherein:
R^{1a} is —H or optionally joined with R^{2a} to form a fused ring selected from the group consisting of five to ten membered aryl, heteroaryl or heterocyclyl rings, said heteroaryl or said heterocyclyl rings having one to three heteroatoms where zero to three of said heteroatoms are N and zero to 1 of said heteroatoms are O or S and where said fused ring is optionally substituted by one to three of R^{9a}, where R^{2a} and R^{9a} are as defined below;
R^{2a} and R^{3a} are independently —H, Het, aryl, C₁₋₁₂ aliphatic, CN, NO₂, halo, R^{10a}, —OR^{10a}, —SR^{10a}, —S(O)R^{10a}, —SO₂R^{10a}, —NR^{10a}R^{11a}, —NR^{11a}R^{12a}, NR^{12a}COR^{13a}, —NR^{12a}CO₂R^{11a}, NR^{12a}CONR^{11a}R^{12a}, —NR^{12a}SO₂R^{11a}, NR^{12a}C(NR^{12a})NHR^{11a}, —COR^{11a}, —CO₂R^{11a}, —CONR^{12a}R^{11a}, —SO₂NR^{12a}R^{11a}, —OCONR^{12a}R^{11a}, C(NR^{12a})NR^{12a}R^{11a} where said C₁₋₁₂ aliphatic optionally bears one or two insertions of one to two groups selected from C(O), O, S, S(O), SO₂ or NR^{12a}; with said Het, aryl or C₁₋₁₂ aliphatic being optionally substituted by one to three of R^{10a}; and where R^{2a} is optionally joined with R^{3a} to form a fused ring selected from the group consisting of five to ten membered aryl, heteroaryl or heterocyclyl rings, said heteroaryl or said heterocyclyl rings having zero to three heteroatoms where zero to three of said heteroatoms are N and zero to one of said heteroatoms are O or S and where said fused ring is optionally substituted by one to three of R^{9a}, where Het, R^{9a},
R^{10a}, R^{11a} and R^{12a} are as defined below;
R^{4a} is —H, halo, —NO₂ or —CN;
R^{5a} is —H or C₁₋₁₂ aliphatic optionally substituted by one to three of halo, hydroxyl, heteroaryl, or aryl;
R^{6a} and R^{7a} are independently halo, —CN, —NO₂, —CONR^{10a}R^{11a}, —SO₂NR^{11a}R^{12a}, —NR^{10a}R^{11a}, or —OR^{11a}, where R^{10a} and R^{11a} are as defined below;
R^{8a} is —OH, —NHSO₂R^{12a} or —NHCOCF₃;
R^{9a} is each independently halo, C₁₋₁₂ aliphatic, —CN, —NO₂, R^{10a}, —OR^{11a}, —SR^{11a}, —S(O)R^{10a}, —SO₂R^{10a}, NR^{10a}R^{11a}, N^{11a}R^{12a}, —NR^{12a}COR^{11a}, —NR^{12a}CO₂R^{11a}, —NR^{12a}CONR^{11a}R^{12a}, —NR^{12a}SO₂R^{11a}, —NR^{12a}C(NR^{12a})NHR^{11a}, —CO₂R^{11a}, CONR^{12a}R^{11a}, SO₂NR^{12a}R^{11a}, —OCONR^{12a}R^{11a} or C(NR^{12a})NR^{12a}R^{11a}, where R^{10a}, R^{11a} and R^{12a} are as defined below;
R^{10a} is each independently —H, halo, C₁₋₁₂ aliphatic, aryl or Het, where said C₁₋₁₂ aliphatic optionally bears an inserted one to two groups selected from O, S, S(O), SO₂ or NR^{12a}, where said C₁₋₁₂ aliphatic, aryl or Het is optionally substituted by one to three of halo, another Het, aryl, —CN, —SR^{12a}, —OR^{12a}, —N(R^{12a})₂, —S(O)R^{12a}, —SO₂R^{12a}, —SO₂N(R^{12a})₂, —NR^{12a}COR^{12a}, —NR^{12a}CO₂R^{12a}, —NR^{12a}CON(R^{12a})₂, —NR^{12a}(NR^{12a})NHR^{12a}, —CO₂R^{12a}, —CON(R^{12a})₂, —NR^{12a}SO₂R^{12a}, —OCON(R^{12a})₂, where Het and R^{12a} are as defined below;
R^{11a} is —H or R^{10a};
R^{12a} is —H, C₁₋₁₂ aliphatic or Het, said C₁₋₁₂ aliphatic optionally substituted by one to three of halo or —OH where Het is as defined below; and
Het is selected from the group consisting of benzofuran, benzoxazole dioxin, dioxane, dioxolane, dithiane, dithiazine, dithiazole, dithiolane, furan, imidazole, indole, indazole, morpholine, oxazole, oxadiazole, oxathiazole, oxathiazolidine, oxazine, oxiadiazine, piperazine, piperidine, pyran, pyrazine, pyrazole, pyridine, pyrimidine, pyrrole, pyrrolidine, quinoline, quinazoline, tetrahydrofuran, tetrazine, tetrazole, thiophene, thiadiazine, thiadiazole, thiatriazole, thiazine, thiazole, thiomorpholine, thianaphthalene, thiopyran, triazine, and triazole;
   and salts, solvates, or physiologically functional derivatives thereof.

A preferred group of compounds of the present invention are those of the general formula (IV) above wherein
R^{1a} is —H or optionally joined with R^{2a} to form a fused ring selected from the group as defined for Het below, and where said fused ring is optionally substituted by one to three of R^{9a}, where R^{2a} and R^{9a} are as defined below;
R^{2a} and R^{2a} are independently —H, Het, aryl, C₁₋₆ aliphatic, —CN, —NO₂, halo, R^{10a}, —OR^{10a}, —SR¹⁰, —S(O)R^{1Oa}, SO₂R^{10a}, —NR^{10a}R^{11a}, —NR^{11a}R^{12a}, —NR^{12a}COR^{11a}, —NR^{12a}CO₂R^{11a}, NR^{12a}CONR^{11a}R^{12a}, —NR^{12a}SO₂R^{11a}, NR^{12a}C(NR^{12a})NHR^{11a}, —COR^{11a}, —CO₂R^{11a}, —CONR^{12a}R^{11a}, —SO₂NR^{12a}R^{11a}, —OCONR^{12a}R^{11a}, C(NR^{11a})N^{12a}R^{11a} where said C₁₋₆ aliphatic optionally bears one or two insertions of one to two groups selected from C(O), O, S, S(O), SO₂ or NR^{12a}; with said Het, aryl or C₁₋₆ aliphatic being optionally substituted by one to three of R^{10a}; and where R^{2a} is optionally joined with R^{3a} to form a fused ring selected from the group as defined below and where said fused ring is optionally substituted by one to three of R^{9a}, where Het, R^{9a}, R^{10a}, R^{11a} and R^{12a} are as defined below;
R^{4a} is —H, halo, —NO₂ or —CN;
R^{5a} is —H or C₁₋₆ aliphatic optionally substituted by one to three of halo, —OH, or aryl;
R^{6a} and R^{7a} are independently halo, —CN, —NO₂, —CONR^{10a}R^{11a}, —SO₂NR^{10a}R^{11a}, —NR^{10a}R^{11a}, or —OR^{11a}, where R^{10a} and R^{11a} are as defined below;
R^{8a} is —OH, —NHSO₂R^{12a} or —NHCOCF₃;
R^{9a} is each independently halo, C₁₋₆ aliphatic, —CN, —NO₂, R^{10a}, —OR^{11a}, —SR^{11a}, —S(O)R^{10a}, SO₂R^{10a}, —NR^{10a}R^{11a}, N^{11a}R^{12a}, —NR^{12a}COR^{11a}, —NR^{12a}CO₂R^{11a}, —NR^{12a}CONR^{11a}R^{12a}, —NR^{12a}SO₂R^{11a}, —NR^{12a}C(NR^{12a})NHR^{11a}, CO₂R^{11a}, CONR^{12a}R^{11a}, —SO₂NR^{12a}R^{11a}, —OCONR^{12a}R^{11a} or C(NR^{12a})NR^{12a}R^{11a}, where R^{10a}, R^{11a} and R^{12a} are as defined below;
R^{10a} is each independently —H, halo, C₁₋₆ aliphatic, aryl or Het, where said C₁₋₆ aliphatic optionally bears an inserted one to two groups selected from O, S, S(O), SO₂ or NR^{12a}, where said C₁₋₆ aliphatic, aryl or Het is optionally substituted by one to three of halo, another Het, aryl, —CN, —SR^{12a}, —OR^{12a}, —N(R^{12a})₂, —S(O)R^{12a}, —SO₂N(R^{12a})₂, —NR^{12a}COR^{12a}, —NR^{12a}CO₂R^{12a}, —NR^{12a}CON(R^{12a})₂, —NR^{12a}(NR^{12a})NHR^{12a}, —CO₂R^{12a}, —CON(R^{12a})₂, —NR^{12a}SO₂R^{12a}, —OCON(R^{12a})₂, where Het and R^{12a} are as defined below;
R^{11a} is —H or R^{10a};
R^{12a} is —H, C₁₋₆ aliphatic or Het, said C₁₋₆ aliphatic optionally substituted by one to three of halo or —OH where Het is as defined below; and
Het is selected from the group consisting of benzofuran, benzoxazole, dioxin, dioxane dioxolane, dithiane, dithiazine, dithiazole, dithiolane, furan, imidazole, indole, indazole, morpholine, oxazole, oxadiazole, oxathiazole, oxathiazolidine, oxazine, oxiadiazine, piperazine, piperidine, pyran, pyrazine, pyrazole, pyridine, pyrimidine, pyrrole, pyrrolidine, quinoline, quinazoline, tetrahydrofuran, tetrazine, tetrazole, thiophene, thiadiazine, thiadiazole, thiatriazole, thiazine, thiazole, thiomorpholine, thianaphthalene, thiopyran, triazine, and triazole;
   and salts, solvates, or physiologically functional derivatives thereof.

A highly preferred group of compounds of the present invention are those of the general formula (IV) wherein
R^{1a} is —H or optionally joined with R^{2a} to form a fused ring selected from the group consisting of fused pyridine, fused triazole, fused thiazole or fused amino-substituted thiazole;
R^{2a} and R^{3a} are independently —H, Het, aryl, C₁₋₆ aliphatic, —R^{12a}NH₂, —R^{12a}-halo, —CN, —NO₂, halo, R^{10a}, —OR^{10a}, —SR^{10a}, —S(O)R^{10a}, —SO₂R^{10a}, —NR^{11a}R^{12a}, —NR^{12a}COR^{11a}, —NR^{12a}CO₂R^{11a}, —NR^{12a}CONR^{11a}R^{12a}, —NR^{12a}SO₂R^{11a}, —NR^{12a}C(NR^{12a})NHR^{11a}, —COR^{11a}, —COR^{11a}NR^{12a}R^{11a}—CO₂R^{11a}, —CONR^{12a}R^{11a}, —SO₂NR^{12a}R^{11a}, —OCONR^{12a}R^{11a}, —C(NH)R^{11a}, —C(NR^{12a})NR^{12a}R^{11a} where said C₁₋₆ aliphatic optionally bears an insertion of a C(O) group; with said Het, aryl or C₁₋₆ aliphatic being optionally substituted by one to three of R^{10a}; and where R^{2a} is optionally joined with R^{3a} to form a fused ring selected from the group as defined for Het below and where said fused ring is optionally substituted by one to three of R^{9a}, where Het, R^{9a}, R^{10a}, R^{11a} and R^{12a} are as defined below;
R^{4a} is H, halo, —NO₂ or —CN;
R^{5a} is —H or C₁₋₆ aliphatic optionally substituted by one to three of halo, —OH, or aryl;
R^{6a} and R^{7a} are independently halo, —CN, —NO₂, —CONR^{10a}R^{11a}, —SO₂NR^{10a}R^{11a}, —NR^{10a}R^{11a}, or —OR^{11a}, where R^{10a} and R^{11a} are as defined below;
R^{8a} is —OH, —NHSO₂R^{12a} or —NHCOCF₃;
R^{9a} is each independently halo, C₁₋₆ aliphatic, —CN, —NO₂, R^{10a}, —OR^{11a}, —SR^{11a}, —S(O)R^{10a}, —SO₂R^{10a}, —NR^{10a}R^{11a}, —N^{11a}R^{12a}, —NR^{12a}COR^{11a}, —NR^{12a}CO₂R^{11a}, —NR^{12a}CONR^{11a}R^{12a}, —NR^{12a}SO₂R^{11a}, —NR^{12a}C(NR^{12a})NHR^{11a}, —CO₂R^{11a}, CONR^{12a}R^{11a}, —SO₂NR^{12a}R^{11a}, —OCONR^{12a}R^{11a} or C(NR^{12a})NR^{12a}R^{11a}, where R^{10a}, R^{11a} and R^{12a} are as defined below;
R^{10a} is each independently —H, halo, C₁₋₆ aliphatic, aryl or Het, where said C₁₋₆ aliphatic optionally bears an inserted one to two groups selected from O, S, S(O), SO₂ or NR^{12a}, where said C₁₋₆ aliphatic, aryl or Het is optionally substituted by one to three of halo, another Het, aryl, —CN, —NO₂—R^{12a}, —SR^{12a}, —OR^{12a}, —N(R^{12a})₂, —R^{12a}N(R^{12a})₂—S(O)R^{12a}, —SO₂R^{12a}, —SO₂N(R^{12a})₂, —NR^{12a}COR^{12a}, —NR^{12a}CO₂R^{12a}, —NR^{12a}CON(R^{12a})₂. NR^{12a}(NR^{12a})NHR^{12a}, —CO₂R^{12a}, —CON(R^{12a})₂, —NR^{12a}SO₂R^{12a}, —OCON(R^{12a})₂, or trifluoro, where Het and R^{12a} are as defined below;
R^{11a} is —H or R^{10a};
R^{12a} is —H, C₁₋₆ aliphatic, —NO₂, C₁₋₆ alkoxy, halo, aryl or —HET, said C₁₋₆ aliphatic optionally substituted by one to three of halogen or —OH where Het is as defined below;
Het is selected from the group consisting of dioxin, dioxane, dioxolane, dithiane, dithiazine, dithiazole, dithiolane, furan, imidazole, imidazopyridinyl, morpholine, oxazole, oxadiazole, oxathiazole, oxathiazolidine, oxazine, oxiadiazine, piperazine, piperidine, pyran, pyrazine, pyrazole, pyridine, pyrimidine, pyrrole, pyrrolidine, tetrahydrofuran, tetrazine, thiophene, thiadiazine, thiadiazole, thiatriazole, thiazine, thiazole, thiomorpholine, thiopyran, thioxotriazine, triazine, and triazole;
   and salts, solvates, or physiologically functional derivatives thereof.

Also highly preferred are compounds of formula (IV) in which R^{1a} and R^{2a} additionally comprise a fused ring which is methyl substituted fused pyridine.

A group of compounds that are preferred with respect to their substituents at positions R^{6a}, R^{7a} and R^{8a} are compounds of the formula (IV), wherein:
R^{1a} is —H or optionally joined with R^{2a} to form a fused ring selected from the group consisting of five to ten membered aryl, heteroaryl or heterocyclyl rings, said heteroaryl or said heterocyclyl rings having one to three heteroatoms where zero to three of said heteroatoms are N and zero to 1 of said heteroatoms are O or S and where said fused ring is optionally substituted by one to three of R^{9a}, where R^{2a} and R^{9a} are as defined below;
R^{2a} and R^{3a} are independently —H, Het, aryl, C₁₋₁₂ aliphatic, —CN, —NO₂, halo, R^{10a}, —OR^{10a}, —SR^{10a}, —S(O)R^{10a}, —SO₂R^{10a}, —NR^{10a}R^{11a}, NR^{11a}R^{12a}, —NR^{12a}COR^{11a}, —NR^{12a}CO₂R^{11a}, NR^{12a}CONR^{11a}R^{12a}, —NR^{12a}SO₂R^{11a}, —NR^{12a}C(NR^{12a})NHR^{11a}, —COR^{11a}, —CO₂R^{11a}—CONR^{12a}R^{11a}, —SO₂NR^{12a}R^{11a}, —OCONR^{12a}R^{11a}, C(NR^{12a})NR^{12a}R^{11a} where said C₁₋₁₂ aliphatic optionally bears one or two insertions of one to two groups selected from C(O), O, S, S(O), SO₂ or NR^{12a}; with said Het, aryl or C₁₋₁₂ aliphatic being optionally substituted by one to three of R^{10a}; and where R^{2a} is optionally joined with R^{3a} to form a fused ring selected from the group consisting of five to ten membered aryl, heteroaryl or heterocyclyl rings, said heteroaryl or said heterocyclyl rings having zero to three heteroatoms where zero to three of said heteroatoms are N and zero to one of said heteroatoms are O or S and where said fused ring is optionally substituted by one to three of R^{9a}, where Het, R^{9a}, R^{10a}, R^{11a} and R^{12a} are as defined below;
R^{4a} is —H, halo, —NO₂ or —CN;
R^{5a} is —H or C₁₋₁₂ aliphatic optionally substituted by one to three of halo, —OH, or aryl;
R^{6a} and R^{7a} are halo;
R^{8a} is —OH;
R^{9a} is each independently halo, C₁₋₁₂ aliphatic, —CN, —NO₂, R^{10a}, —OR^{11a}, —SR^{11a}, —S(O)R^{10a}, —SO₂R^{10a}, —NR^{10a}R^{11a}, —N^{11a}R^{12a}, —NR^{12a}COR^{11a}, —NR^{12a}CO₂R^{11a}, —NR^{12a}CONR^{11a}R^{12a}, —NR^{12a}SO₂R^{11a}, —NR^{12a}C(NR^{12a})NHR^{11a}, —CO₂R^{11a}, —CONR^{12a}R^{11a}, —SO₂NR^{12a}R^{11a}, —OCONR^{11a}R^{11a} or C(NR^{12a})NR^{12a}R^{11a}, where R^{11a}, R^{11a} and R^{12a} are as defined below;
R^{10a} is each independently —H, halo, C₁₋₁₂ aliphatic, aryl or Het, where said C₁₋₁₂ aliphatic optionally bears an inserted one to two groups selected from O, S, S(O), SO₂ or NR¹², where said C₁₋₁₂ aliphatic, aryl or Het is optionally substituted by one to three of halo, another Het, aryl, —CN, —SR^{12a}, —OR^{12a}, —N(R^{12a})₂, —S(O)R^{12a}, —SO₂R^{12a}, SO₂N(R^{12a})₂, —NR^{12a}COR^{12a}, —NR^{12a}CO₂R^{12a}, —NR^{12a}CON(R^{12a})₂, —NR^{12a}(NR^{12a})NHR^{12a}, —CO₂R^{12a}, —CON(R^{12a})₂, —NR^{12a}SO₂R^{12a}, —OCON(R^{12a})₂, where Het and R^{12a} are as defined below;
R^{11a} is —H or R^{10a};
R^{12a} is —H, C₁₋₁₂ aliphatic or Het, said C₁₋₁₂ aliphatic optionally substituted by one to three of halo or —OH where Het is as defined below; and
Het is selected from the group consisting of benzofuran, benzoxazole, dioxin, dioxane dioxolane, dithiane, dithiazine, dithiazole, dithiolane, furan, imidazole, indole, indazole, morpholine, oxazole, oxadiazole, oxathiazole, oxathiazolidine, oxazine, oxiadiazine, piperazine, piperidine, pyran, pyrazine, pyrazole, pyridine, pyrimidine, pyrrole, pyrrolidine, quinoline, quinazoline, tetrahydrofuran, tetrazine, tetrazole, thiophene, thiadiazine, thiadiazole, thiatriazole, thiazine, thiazole, thiomorpholine, thianaphthalene, thiopyran, triazine, and triazole;
   and salts, solvates, or physiologically functional derivatives thereof.

Another group of compounds that are preferred with respect to their substituents at positions R^{6a}, R^{7a} and R^{8a} are compounds of the formula (IV) wherein:
R^{1a} is —H or optionally joined with R^{2a} to form a fused ring selected from the group consisting of five to ten membered aryl, heteroaryl or heterocyclyl rings, said heteroaryl or said heterocyclyl rings having one to three heteroatoms where zero to three of said heteroatoms are N and zero to 1 of said heteroatoms are O or S and where said fused ring is optionally substituted by one to three of R^{9a}, where R^{2a} and R^{9a} are as defined below;
R^{2a} and R^{3a} are independently —H, Het, aryl, C₁₋₁₂ aliphatic, —CN, —NO₂, halo, R^{10a}, —OR^{10a}, —SR^{10a}, —S(O)R^{10a}, —SO₂R^{10a}, —NR^{10a}R^{11a}, NR^{11a}R^{12a}, —NR^{12a}COR^{12a}, —NR^{12a}CO₂R^{11a}, —NR^{12a}CONR^{11a}R^{12a}, —NR^{12a}SO₂R^{11a}, —NR^{12a}C(NR^{12a})NHR^{11a}, —COR^{11a}, —CO₂R^{11a}, —CONR^{12a}R^{11a}, —SO₂NR^{12a}R^{11a}, —OCONR^{12a}R^{11a}, C(NR^{12a})NR^{12a}R^{11a} where said C₁₋₁₂ aliphatic optionally bears one or two insertions of one to two groups selected from C(O), O, S, S(O), SO₂ or NR^{12a}; with said Het, aryl or C₁₋₁₂ aliphatic being optionally substituted by one to three of R^{10a}; and where R^{2a} is optionally joined with R^{3a} to form a fused ring selected from the group consisting of five to ten membered aryl, heteroaryl or heterocyclyl rings, said heteroaryl or said heterocyclyl rings having zero to three heteroatoms where zero to three of said heteroatoms are N and zero to one of said heteroatoms are O or S and where said fused ring is optionally substituted by one to three of R^{9a}, where Het, R^{9a}, R^{10a}, R^{11a} and R^{12a} are as defined below;
R^{4a} is —H, halo, —NO₂ or —CN;
R^{5a} is —H or C₁₋₁₂ aliphatic optionally substituted by one to three of halo, —OH, or aryl;
R^{6a} and R^{7a} are independently bromo or chloro;
R^{8a} is —OH;
R^{9a} is each independently halo, C₁₋₁₂ aliphatic, —CN, —NO₂, R^{10a}, —OR^{11a}, —SR^{11a}, —S(O)R^{10a}, —SO₂R^{10a}—NR^{10a}R^{11a}, —N^{11a}R^{12a}, —NR^{12a}COR^{11a}, —NR^{12a}CO₂R^{11a}, —NR^{12a}CONR^{11a}R^{12a}, —NR^{12a}SO₂R^{11a}, —NR^{12a}C(NR^{12a})NHR^{11a}, —CO₂R^{11a}, —CONR^{12a}R^{11a}, —SO₂NR^{12a}R^{11a}, —OCONR^{12a}R^{11a} or C(NR^{12a})NR^{12a}R^{11a}, where R^{10a}, R^{11a} and R^{12a} are as defined below;
R^{10a} is each independently —H, halo, C₁₋₁₂ aliphatic, aryl or Het, where said C₁₋₁₂ aliphatic optionally bears an inserted one to two groups selected from O, S, S(O), SO₂ or NR^{12B}, where said C₁₋₁₂ aliphatic, aryl or Het is optionally substituted by one to three of halo, another Het, aryl, —CN, —SR^{12a}, —OR^{12a}, —N(R^{12a})₂, —S(O)R^{12a}, —SO₂R^{12a}, —SO₂N(R^{12a})₂, —NR^{12a}COR^{12a}, —NR^{12a}CO₂R^{12a}, —NR^{12a}CON(R^{12a})₂, —NR^{12a}(NR^{12a})NHR^{12a}, —CO₂R^{12a}, —CON(R^{12a})₂, —NR^{12a}SO₂R^{12a}, —OCON(R^{12a})₂, where Het and R^{12a} are as defined below;
R^{11a} is —H or R^{10a};
R^{12a} is —H, C₁₋₁₂ aliphatic or Het, said C₁₋₁₂ aliphatic optionally substituted by one to three of halo or —OH where Het is as defined below; and
Het is selected from the group consisting of benzofuran, benzoxazole, dioxin, dioxane dioxolane, dithiane, dithiazine, dithiazole, dithiolane, furan, imidazole, indole, indazole, morpholine, oxazole, oxadiazole, oxathiazole, oxathiazolidine, oxazine, oxiadiazine, piperazine, piperidine, pyran, pyrazine, pyrazole, pyridine, pyrimidine, pyrrole, pyrrolidine, quinoline, quinazoline, tetrahydrofuran, tetrazine, tetrazole, thiophene, thiadiazine, thiadiazole, thiatriazole, thiazine, thiazole, thiomorpholine, thianaphthalene, thiopyran, triazine, and triazole;
   and salts, solvates, or physiologically functional derivatives thereof.

Yet another group of compounds that are preferred with respect to their substituents at positions R^{6a}, R^{7a} and R^{8a} are compounds of the formula (IV) wherein:
R^{1a} is —H or optionally joined with R^{2a} to form a fused ring selected from the group consisting of five to six membered heteroaryl rings, said heteroaryl ring having one to two heteroatoms where zero to two of said heteroatoms are N and zero to two of said heteroatoms are O or S and where said fused ring is optionally substituted by one to three of R^{9a}, where R^{2a} and R^{9a} are as defined below;
R^{2a} and R^{3a} are independently —H, Het, phenyl, C₁₋₆ aliphatic, —NR^{10a}R^{11a}, —COR^{11a}, —CO₂R^{11a}, —CONR^{12a}R^{11a}, —SO₂NR^{12a}R^{11a}, with said Het, phenyl or C₁₋₆ aliphatic being optionally substituted by R^{10a}; and where R^{2a}, is optionally joined with R^{3a} to form a fused five membered heterocyclyl ring, said heterocyclyl ring having zero to 1 heteroatoms where said heteroatom is N and zero to 1 heteroatoms where said heteroatoms are O or S and where said fused ring is optionally substituted by R^{9a}, where Het, R^{9a}, R^{10a}, R^{11a} and R^{12a} are as defined below;
R^{4a} is —H;
R^{5a} is H;
R^{6a} and R^{7a} are independently bromo or chloro;
R^{8a} is —OH;
R^{9a} is —H, C₁₋₆ aliphatic, or —COR^{10a}, where R^{10a} is as defined below;
R^{10a} is —H, C₁₋₆ aliphatic or amino;
R^{11a} is —H, C₁₋₆ aliphatic, hydroxy-C₁₋₆ aliphatic, phenyl, phenyl-C₁₋₆ aliphatic or Het;
R^{12a} is —H, C₁₋₆ aliphatic, hydroxy-C₁₋₆ aliphatic or (R^{11a})₂N-C₁₋₆aliphatic; and
Het is selected from the group consisting of oxazole, pyridine, tetrazole and thiazole;
   and salts, solvates, or physiologically functional derivatives thereof.

Still another group of compounds that are preferred with respect to their substituents at positions R^{6a}, R^{7a} and R^{8a} are compounds of the formula (IV) wherein:
R^{1a} is —H;
R^{2a} and R^{3a} are independently —H, Het, phenyl, C₁₋₆ aliphatic, —CN, halo, —COR^{11a}, or —CONR^{12a}R^{11a}, with said Het, phenyl or C₁₋₆ aliphatic being optionally substituted by R^{10a}, where Het, R^{10a}, R^{11a} and R^{12a} are as defined below;
R^{4a} is —H;
R^{5a} is —H;
R^{6a} and R^{7a} are independently bromo or chloro;
R^{8a} is —OH;
R^{10a} is —H, C₁₋₆ aliphatic, oxo or —CN;
R^{11a} is —H, C₁₋₆ aliphatic, trihalo-C₁₋₆ aliphatic, phenyl or nitro-substituted phenyl;
R^{12a} is —H, C₁₋₆ aliphatic, hydroxy-C₁₋₆ aliphatic; and
Het is thiophene or pyridine;
   and salts, solvates, or physiologically functional derivatives thereof.

Due to the presence of a double bond, also included in the compounds of the invention are their respective pure E and Z geometric isomers as well as mixtures of E and Z isomers.

A group of preferred species of compounds of formula (IV) comprises the group: 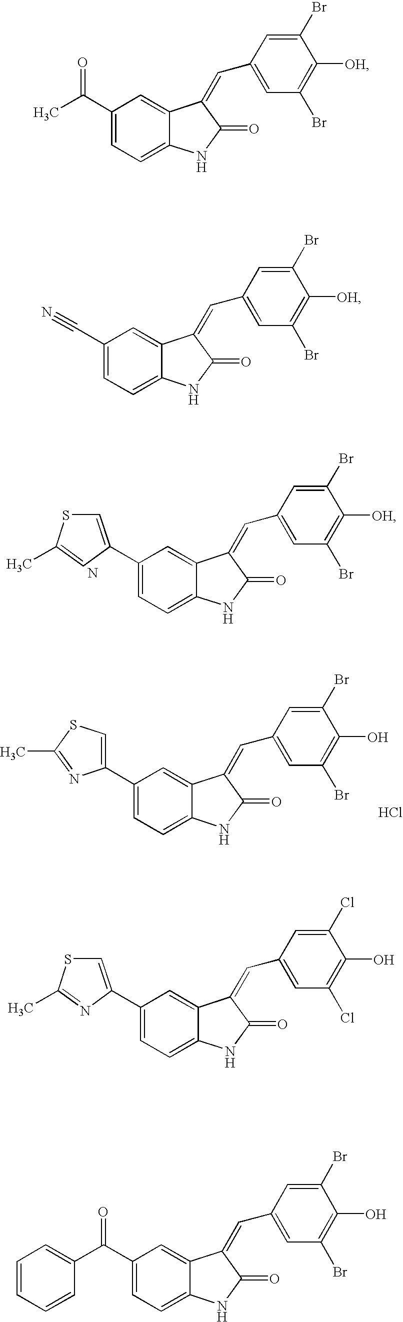 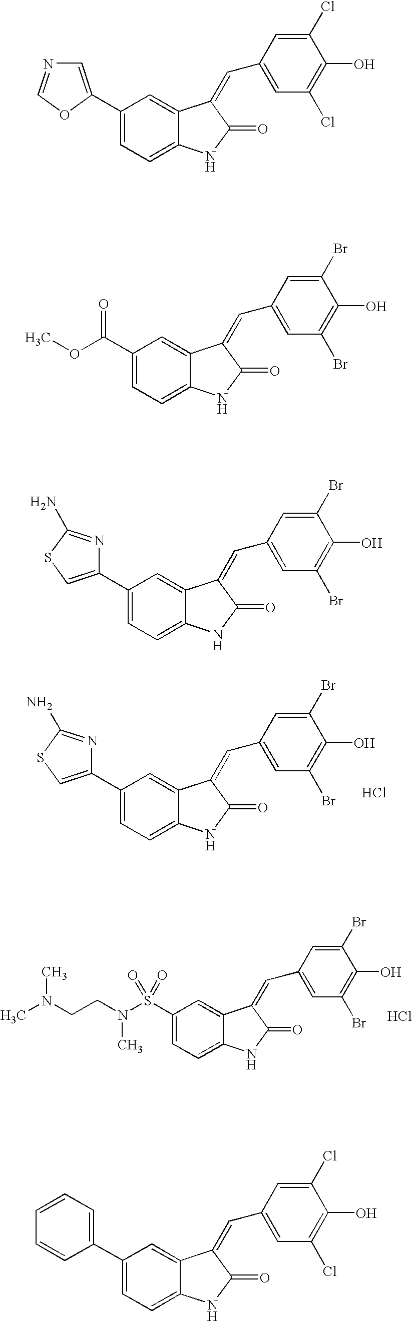 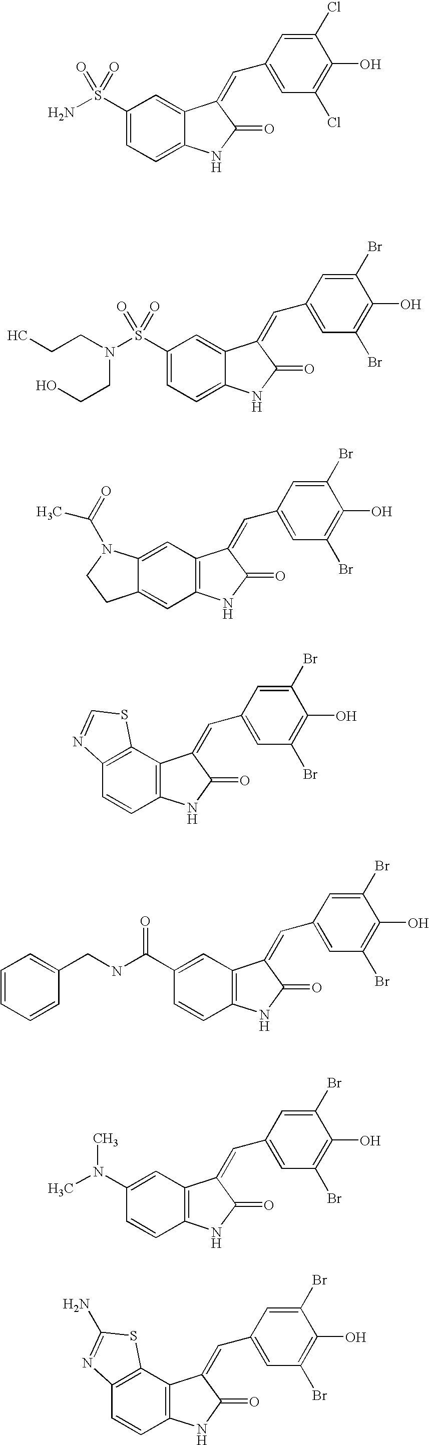 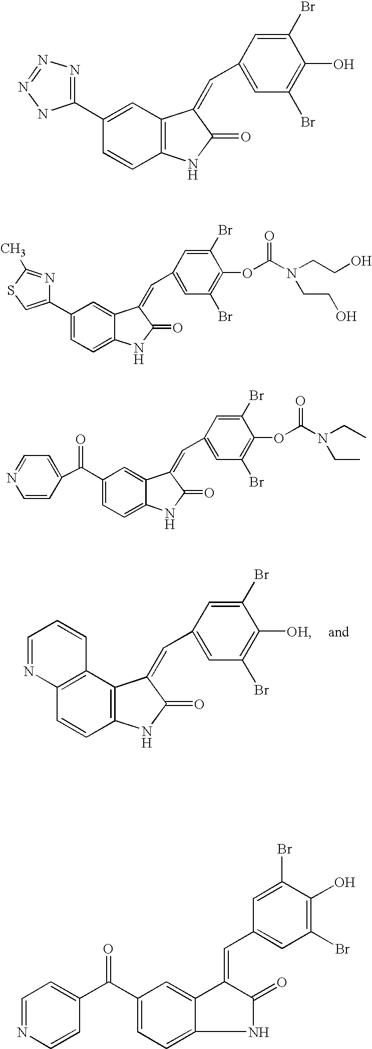
Another group of preferred compounds of the invention comprises the group: 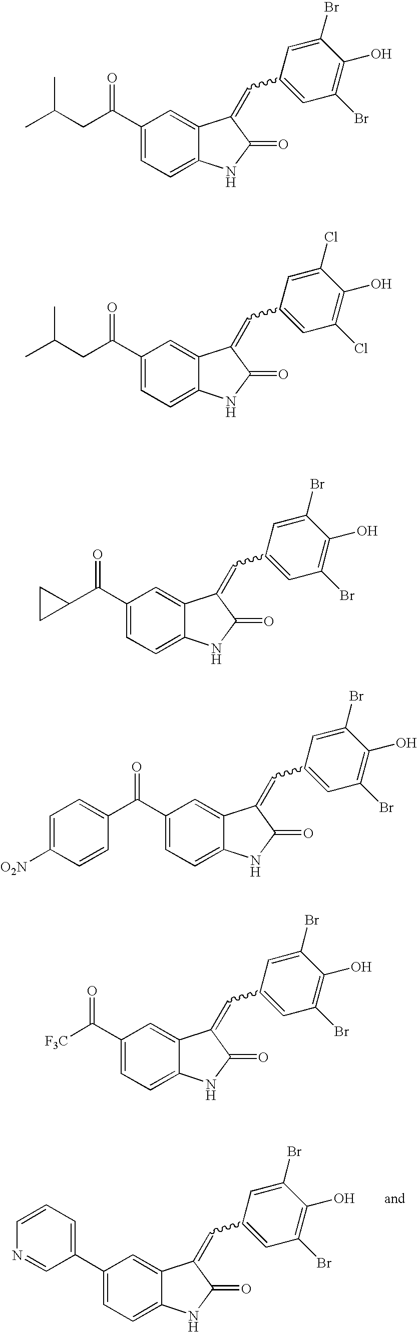 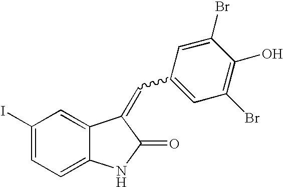
Still another group of preferred compounds comprises the group: 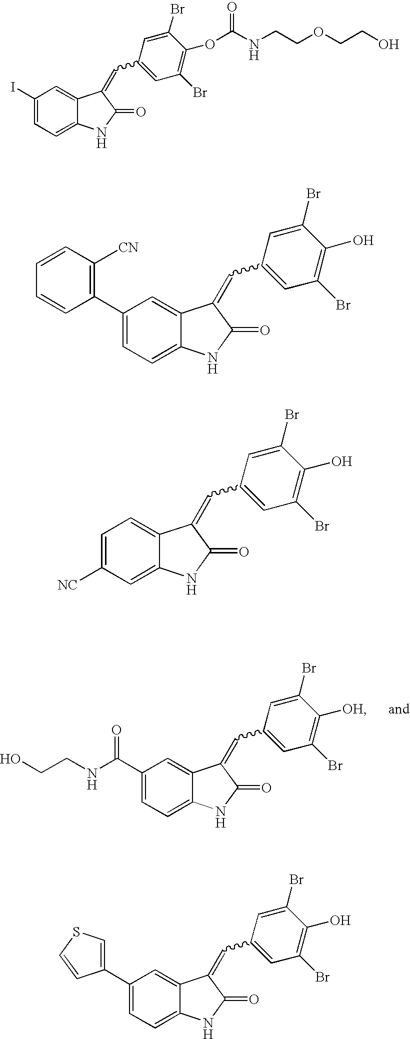
An especially preferred group of compounds comprises the group:

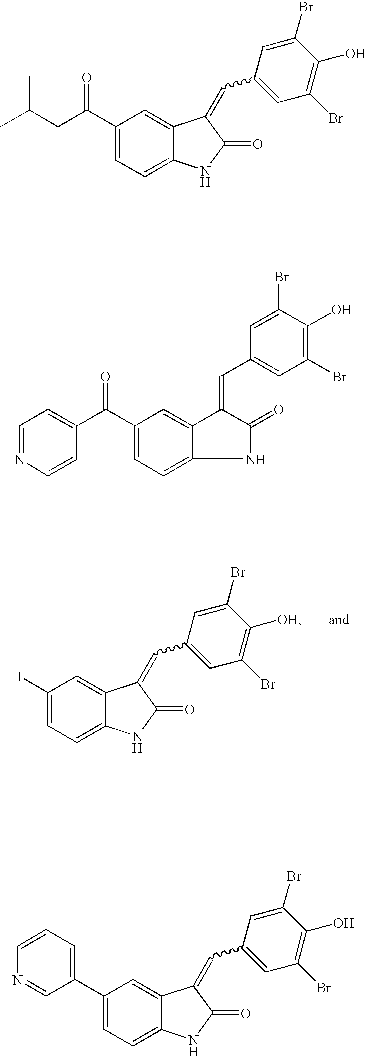

R^{1a} is —H. Optionally, R^{1a} can be joined with an R^{2a} substituent to form a fused ring. Such fused rings can be five to ten membered aryl, heteroaryl, or heterocyclyl rings or ring systems, having 1 to 3 heteroatoms. These heteroatoms can be nitrogen, oxygen or sulfur. Such fused rings can be optionally substituted by one to three groups of halo, cyano, nitro, substituted amide, substituted sulfonamide, substituted amine, substituted ether or hydroxyl. Substitutents for amides, sulfonamides, amines, or ethers include hydrogen, halogen, 1 to 12 carbon aliphatic (which can bear an inserted group anywhere along its chain length of an oxygen, a sulfur, a sulfoxide, a sulfone, a sulfine, or a secondary amine), aryl rings, heterocyclic rings. Substituents on these aliphatic, aryl or heterocyclic groups include 1 to 3 substitutions by a halogen, another heterocylic ring, another aryl ring, cyano, substituted sulfo, substituted oxy, substituted amine, substituted sulfoxide, substituted sulfine, substituted sulfone, substituted sulfonamide, substituted amide, substituted ureide, substituted ester, substituted carbamate. These substituents in turn can be 1 to 12 carbon aliphatic or a heterocyclic ring, where the 1 to 12 carbon aliphatic itself can be substituted by 1 to 3 occurences of a halo, or hydroxyl.

Alternatively, R^{1a} can be —H or optionally, R^{1a} can be joined with an R^{2a} substituent to form a fused ring. Such fused rings can be from the group comprising benzofuran, benzoxazole, dioxin, dioxane, dioxolane, dithiane, dithiazine, dithiazole, dithiolane, furan, imidazole, indole, indazole, morpholine, oxazole, oxadiazole, oxathiazole, oxathiazolidine, oxazine, oxiadiazine, piperazine, piperidine, pyran, pyrazine, pyrazole, pyridine, pyrimidine, pyrrole, pyrrolidine, quinoline, quinazoline, tetrahydrofuran, tetrazine, tetrazole, thiophene, thiadiazine, thiadiazole, thiatriazole, thiazine, thiazole, thiomorpholine, thianaphthalene, thiopyran, triazine, and triazole. Any of these rings can in turn be substituted by a group from the substituents comprising 1 to 3 substitutions by a halo, another heterocylic ring, another aryl ring, cyano, substituted sulfo, substituted oxy, substituted amine, substituted sulfoxide, substituted sulfine, substituted sulfone, substituted sulfonamide, substituted amide, substituted ureide, substituted ester, or substituted carbamate. These substituents in turn can be 1 to 12 carbon aliphatic or a heterocyclic ring, where the 1 to 12 carbon aliphatic itself can be substituted by 1 to 3 occurences of a halo, or hydroxyl.

Preferably, R^{1a} is —H or fused with R^{2a} to form fused pyridine, fused triazole, fused thiazole or fused amino-substituted thiazole.

Most preferably, R^{1a} is —H.

R^{2a} is —H, an aryl ring, a heterocyclic ring, a 1 to 12 carbon aliphatic, cyano, nitro, halo, substituted ether, substituted thioether, substitued sulfine, substituted sulfone, substituted amine, disubstituted amine, substituted amide, substituted carbamate, substituted sulfonamide, substituted carbonyl, or substituted ester. These substituents can be hydrogen, halogen, 1 to 12 carbon aliphatic (which can bear an inserted group anywhere along its chain length of an oxygen, a sulfur, a sulfoxide, a sulfone, a sulfine, or a secondary amine), aryl rings, heterocyclic rings. Substituents on these aliphatic, aryl or heterocyclic groups include 1 to 3 substitutions by a halo, another heterocylic ring, another aryl ring, cyano, substituted sulfo, substituted oxy, substituted amine, substituted sulfoxide, substituted sulfine, substituted sulfone, substituted sulfonamide, substituted amide, substituted ureide, substituted ester, substituted carbamate. These substituents in turn can be 1 to 12 carbon aliphatic or a heterocyclic ring, where the 1 to 12 carbon aliphatic itself can be substituted by 1 to 3 occurences of a halo, or hydroxyl.

R^{2a} can be joined with R^{3a} to form a fused ring selected from the group comprising benzofuran, benzoxazole, dioxin, dioxane, dioxolane, dithiane, dithiazine, dithiazole, dithiolane, furan, imidazole, indole, indazole, morpholine, oxazole, oxadiazole, oxathiazole, oxathiazolidine, oxazine, oxiadiazine, piperazine, piperidine, pyran, pyrazine, pyrazole, pyridine, pyrimidine, pyrrole, pyrrolidine, quinoline, quinazoline, tetrahydrofuran, tetrazine, tetrazole, thiophene, thiadiazine, thiadiazole, thiatriazole, thiazine, thiazole, thiomorpholine, thianaphthalene, thiopyran, triazine, and triazole.

R^{2a} can more preferably be —H, a heterocyclic ring, phenyl, a 1 to 6 carbon aliphatic, a substituted amine, a substituted carbonyl, a substituted ester, a substituted amide, or a substituted sulfonamide. Said heterocyclic ring, phenyl or aliphatic group are optionally substituted by amino or 1 to 6 carbon aliphatic. Said amine, carbonyl, ester amide or sulfonamide are optionally substituted by 1 to 6 carbon aliphatic, amino, hydroxy-aliphatic of 1 to 6 carbons, phenyl, phenyl-aliphatic of 1 to 6 carbons, amino-aliphatic of 1 to 12 carbons or heterocyclic rings such as oxazole, pyridine, tetrazole or thiazole.

R^{2a} can more preferably be joined with R^{3a} to form a five membered fused ring having a heteroatom of either nitrogen, oxygen or sulfur. These fused rings can be substituted by 1 to 6 carbon aliphatic, or a 1 to 6 carbon acyl group.

R^{2a} can also more preferably be —H, thiophene, pyridine, phenyl, 1 to 6 carbon aliphatic, cyano, halo, substituted acyl, or substituted amide. These substituents can be 1 to 6 carbon aliphatic, tri-halogen 1 to 6 carbon aliphatic, phenyl, nitro-substituted phenyl, or hydroxy-aliphatic of 1 to 6 carbons.

R^{2a} is —H, an aryl ring, a heterocyclic ring, a 1 to 12 carbon aliphatic, cyano, nitro, halo, substituted ether, substituted thioether, substitued sulfine, substituted sulfone, substituted amine, disubstituted amine, substituted amide, substituted carbamate, substituted sulfonamide, substituted carbonyl, or substituted ester. These substituents can be hydrogen, halogen, 1 to 12 carbon aliphatic (which can bear an inserted group anywhere along its chain length of an oxygen, a sulfur, a sulfoxide, a sulfone, a sulfine, or a secondary amine), aryl rings, heterocyclic rings. Substituents on these aliphatic, aryl or heterocyclic groups include 1 to 3 substitutions by a halo, another heterocylic ring, another aryl ring, cyano, substituted sulfo, substituted oxy, substituted amine, substituted sulfoxide, substituted sulfine, substituted sulfone, substituted sulfonamide, substituted amide, substituted ureide, substituted ester, substituted carbamate. These substituents in turn can be 1 to 12 carbon aliphatic or a heterocyclic ring, where the 1 to 12 carbon aliphatic itself can be substituted by 1 to 3 occurences of a halo, or hydroxyl.

R^{3a} can be joined with R^{2a} to form a fused ring selected from the group comprising benzofuran, benzoxazole, dioxin, dioxane, dioxolane, dithiane, dithiazine, dithiazole, dithiolane, furan, imidazole, indole, indazole, morpholine, oxazole, oxadiazole, oxathiazole, oxathiazolidine, oxazine, oxiadiazine, piperazine, piperidine, pyran, pyrazine, pyrazole, pyridine, pyrimidine, pyrrole, pyrrolidine, quinoline, quinazoline, tetrahydrofuran, tetrazine, tetrazole, thiophene, thiadiazine, thiadiazole, thiatriazole, thiazine, thiazole, thiomorpholine, thianaphthalene, thiopyran, triazine, and triazole.

R^{3a} can more preferably be —H, a heterocyclic ring, phenyl, a 1 to 6 carbon aliphatic, a substituted amine, a substituted carbonyl, a substituted ester, a substituted amide, or a substituted sulfonamide. Said heterocyclic ring, phenyl or aliphatic group are optionally substituted by amino or 1 to 6 carbon aliphatic. Said amine, carbonyl, ester amide or sulfonamide are optionally substituted by 1 to 6 carbon aliphatic, amino, hydroxy-aliphatic of 1 to 6 carbons, phenyl, phenyl-aliphatic of 1 to 6 carbons, amino-aliphatic of 1 to 12 carbons or heterocyclic rings such as oxazole, pyridine, tetrazole or thiazole.

R^{3a} can more preferably be joined with R^{2a} to form a five membered fused ring having a heteroatom of either nitrogen, oxygen or sulfur. These fused rings can be substituted by 1 to 6 carbon aliphatic, or a 1 to 6 carbon acyl group.

R^{3a} can also more preferably be —H, thiophene, pyridine, phenyl, 1 to 6 carbon aliphatic, cyano, halo, substituted acyl, or substituted amide. These substituents can be 1 to 6 carbon aliphatic, tri-halo 1 to 6 carbon aliphatic, phenyl, nitro-substituted phenyl, or hydroxy-aliphatic of 1 to 6 carbons.

R^{4a} is —H, nitro, cyano, or halo.

Preferably, R^{4a} is —H.

R^{5a} is —H or 1 to 12 carbon aliphatic, which is optionally substituted at 1 to 3 positions by a halo, hydroxyl, or an aryl ring.

R^{5a} is alternatively —H or 1 to 6 carbon aliphatic, which is optionally substituted at 1 to 3 positions by a halo, hydroxyl or an aryl ring.

Preferably, R^{5a} is —H.

R^{6a} is halo, cyano, nitro, substituted amide, substituted sulfonamide, substituted amine, substituted ether or hydroxyl. Substitutents for amides, sulfonamides, amines, or ethers include hydrogen, halogen, 1 to 12 carbon aliphatic (which can bear an inserted group anywhere along its chain length of an oxygen, a sulfur, a sulfoxide, a sulfone, a sulfine, or a secondary amine), aryl rings, heterocyclic rings. Substituents on these aliphatic, aryl or heterocyclic groups include 1 to 3 substitutions by a halo, another heterocylic ring, another aryl ring, cyano, substituted sulfo, substituted oxy, substituted amine, substituted sulfoxide, substituted sulfine, substituted sulfone, substituted sulfonamide, substituted amide, substituted ureide, substituted ester, substituted carbamate. These substituents in turn can be 1 to 12 carbon aliphatic or a heterocyclic ring, where the 1 to 12 carbon aliphatic itself can be substituted by 1 to 3 occurences of a halo, or hydroxyl.

R^{6a} is more preferably a halo.

R^{6a} is most preferably a bromo.

Alternatively, R^{6a} is most preferably a chloro.

R^{7a} is halo, cyano, nitro, substituted amide, substituted sulfonamide, substituted amine, substituted ether or hydroxyl. Substitutents for amides, sulfonamides, amines, or ethers include hydrogen, halogen, 1 to 12 carbon aliphatic (which can bear an inserted group anywhere along its chain length of an oxygen, a sulfur, a sulfoxide, a sulfone, a sulfine, or a secondary amine), aryl rings, heterocyclic rings. Substituents on these aliphatic, aryl or heterocyclic groups include 1 to 3 substitutions by a halo, another heterocylic ring, another aryl ring, cyano, substituted sulfo, substituted oxy, substituted amine, substituted sulfoxide, substituted sulfine, substituted sulfone, substituted sulfonamide, substituted amide, substituted ureide, substituted ester, substituted carbamate. These substituents in turn can be 1 to 12 carbon aliphatic or a heterocyclic ring, where the 1 to 12 carbon aliphatic itself can be substituted by 1 to 3 occurences of a halo, or hydroxyl.

R^{7a} is more preferably a halo.

R^{7a} is most preferably a bromo.

Alternatively, R^{7a} is most preferably a chloro.

R^{8a} is hydroxy or sulfonamide optionally substituted by a 1 to 12 carbon aliphatic, or substituted by a heterocyclic ring. This aliphatic group itself can be substituted by 1 to 3 halos or hydroxy.

Alternatively, R^{8a} is hydroxy or —NHCOCF₃, or sulfonamide optionally substituted by a 1 to 6 carbon aliphatic, or substituted by a heterocyclic ring.

Or, R^{8a} is hydroxy or —NHCOCF₃, or sulfonamide optionally substituted by a 1 to 6 carbon aliphatic, a nitro, a 1 to 6 carbon alkoxy, a halo, an aryl or a hetercyclic ring. This aliphatic group itself be substituted by 1 to 3 halos or hydroxy.

Preferably, R^{8a} is hydroxy.

The compounds of Formula (IV) may be prepared according to the procedures of U.S. patent application Ser. No. 09/446,586, filed Apr. 7, 2000, and issued as U.S. Pat. No. 6,268,391 on Jul. 31, 2001.

In another embodiment, the Raf family inhibitor may be a bRaf inhibitor. Generally any bRaf inhibitor, that is any pharmaceutical agent having specific bRaf inhibitor activity may be utilized in the present invention. Such bRaf inhibitors are described, for instance, in International Patent Applications WO 02/24680 and WO 03/022840 which patent applications are herein incorporated by reference to the extent of their disclosure of bRaf inhibitor compounds and methods of making and using the same.

One class of bRaf inhibitor compounds that may be usefully employed in the present invention includes compounds of the Formula V: 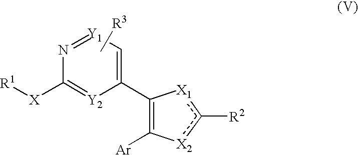
wherein
X is O, CH₂, CO, S or NH, or the moiety X—R¹ is hydrogen;
Y₁ and Y₂ are independently N or CH;
R¹ is hydrogen, C₁₋₆alkyl, C₃₋₇cycloalkyl, aryl, arylC₁₋₆alkyl, heterocyclyl, heterocyclylC₁₋₆alkyl, heteroaryl, or heteroarylC₁₋₆alkyl, any of which may be optionally substituted; in addition when X is CH₂ then R¹ may be hydroxy or C₁₋₆alkoxy which may be optionally substituted;
R² is H, C₁₋₆alkyl, C₂₋₆alkenyl, C₃₋₇cycloalkyl, C₅₋₇cycloalkenyl, heterocyclyl, aryl or heteroaryl, any of which may be optionally substituted;
Ar is a group of the formula a) or b): 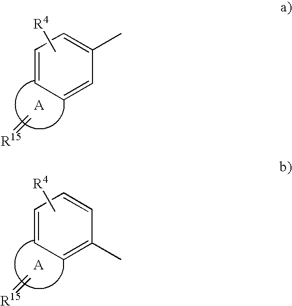
wherein A represents a fused 5- to 7-membered ring optionally containing up to two heteroatoms selected from O, S and NR⁵, wherein R⁵ is hydrogen or C₁₋₆alkyl, which ring is optionally substituted by up to 2 substituents selected from halogen, C₁₋₆alkyl, hydroxy, C₁₋₆alkoxy or keto;
R³ and R⁴ are independently selected from hydrogen, halogen, C₁₋₆alkyl, aryl, aryl C₁₋₆alkyl, C₁₋₆alkoxy, C₁₋₆alkoxyC₁₋₆alkyl, haloC₁₋₆alkyl, arylC₁₋₆alkoxy, hydroxy, nitro, cyano, azido, amino, mono- and di-N-C₁₋₆alkylamino, acylamino, arylcarbonylamino, acyloxy, carboxy, carboxy salts, carboxy esters, carbamoyl, mono- and di-N-C₁₋₆alkylcarbamoyl, C₁₋₆alkoxycarbonyl, aryloxycarbonyl, ureido, guanidino, C₁₋₆alkylguanidino, amidino, C₁₋₆alkylamidino, sulphonylamino, aminosulphonyl, C₁₋₆alkylthio, C₁₋₆alkylsulphinyl or C₁₋₆alkylsulphonyl;
R¹⁵ is O or N—OH;
one of X₁ and X₂ is N and the other is NR⁶ wherein R⁶ is hydrogen or C₁₋₆alkyl;
or a pharmaceutically acceptable salt thereof.

As used herein, the double bond indicated by the dotted lines of formula (I), represent the possible tautomeric ring forms of the compounds falling within the scope of this invention. It will be understood that the double bond is to the unsubstituted nitrogen.

The oxime moiety can be positioned on any of carbon atoms of the non-aromatic ring in groups a) and b).

Alkyl and alkenyl groups referred to herein, individually or as part of larger groups e.g. alkoxy, may be straight or branched groups containing up to six carbon atoms.

Cycloalkyl and cycloalkenyl groups referred to herein include groups having from three to seven and five to seven ring carbon atoms respectively.

Optional substituents for alkyl, alkenyl, cycloalkyl and cycloalkenyl groups include aryl, heteroaryl, heterocyclyl, C₁₋₆alkoxy, C₁₋₆alkylthio, arylC₁₋₆alkoxy, aryl C₁₋₆alkylthio, amino, mono- or di-C₁₋₆alkylamino, aminosulphonyl, cycloalkyl, cycloalkenyl, carboxy and esters thereof, amide, ureido, guanidino, C₁₋₆alkylguanidino, amidino, C₁₋₆alkylamidino, C₁₋₆acyloxy, hydroxy, and halogen or any combinations thereof. A further substituent can be cyano.

Preferably the optional substituent contains a water-solubilising group; suitable solubilising moieties will be apparent to those skilled in the art and include hydroxy and amino group. Even more preferably the optional substituent includes amino, mono or di-C₁₋₆alkyl, amino, amino containing heterocyclyl or hydroxy or any combination thereof.

Suitably aryl, heterocyclyl and heteroaryl groups may be optionally substituted by preferably up to three substituents. Suitable substituents include halogen, hydroxy, C₁₋₆alkyl, aryl, arylC₁₋₆alkyl, C₁₋₆alkoxy, C₁₋₆alkoxyC₁₋₆alkyl, haloC₁₋₆alkyl, arylC₁₋₆alkoxy, nitro, cyano, azido, amino, mono- and di-N-C₁₋₆alkylamino, acylamino, arylcarbonylamino, acyloxy, carboxy, carboxy salts, carboxy esters, carbamoyl, mono- and di-N-C₁₋₆alkylcarbamoyl, C₁₋₆alkoxycarbonyl, aryloxycarbonyl, ureido, guanidino, C₁₋₆alkylguanidino, amidino, C₁₋₆alkylamidino, urea, carbamate, acyl, sulphonylamino, aminosulphonyl, C₁₋₆alkylthio, C₁₋₆alkylsulphinyl, C₁₋₆alkylsulphonyl, heterocyclyl, heteroaryl, heterocyclylC₁₋₆alkyl and heteroaryl C₁₋₆alkyl, or any combination thereof. In addition two ring carbon atoms may be linked to form a bicyclic system.

In the compounds of formula (V):

X is preferably O. CH₂, S or NH, or the moiety X—R¹ is hydrogen.

More preferably X is CH₂ or NH or X—R¹ is hydrogen, most preferably X is NH or X—R¹ is hydrogen.

Preferably Y₁ is CH and Y₂ is N or CH.

Preferably R¹⁵ is N—OH.

Alternatively, R¹ is hydrogen, C₁₋₆alkyl, aryl, arylC₁₋₆alkyl, heterocyclyl, heterocyclylC₁₋₆alkyl, heteroaryl, or heteroarylC₁₋₆alkyl, any of which may be optionally substituted; in addition when X is CH₂ then R¹ may be hydroxy or C₁₋₆alkoxy which may be optionally substituted.

R² can be C₁₋₆alkyl, C₂₋₆alkenyl, C₃₋₇cycloalkyl, C₅₋₇cycloalkenyl or heterocyclyl, any of which may be optionally substituted.

Alternatively R² is aryl or heteroaryl, either of which may be optionally substituted.

Preferably Ar is a group Ar is a group of the formula a) or b):

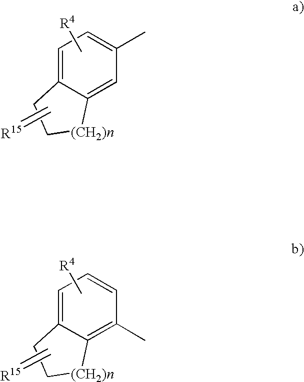

R⁴ is as defined for compounds of formula (I), n is 1, 2 or 3 and R¹⁵ is O or N—OH.

More preferably Ar is a group of formula a) or b) 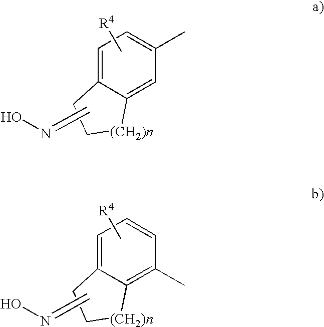
wherein R⁴ is as defined for compounds of formula (I), and n is 1, 2 or 3.

n is preferably 1.

Ar is preferably an indone group.

Suitable optional substituents for the group R² include one or more groups selected from the group consisting of aryl, heteroaryl, heterocyclyl, C₁₋₆alkoxy, C₁₋₆alkylthio, arylC₁₋₆alkoxy, arylC₁₋₆alkylthio, amino, mono- or di-C₁₋₆alkylamino, aminosulphonyl, cycloalkyl, cycloalkenyl, carboxy and esters thereof, amide, ureido, guanidino, C₁₋₆alkylguanidino, amidino, C₁₋₆alkylamidino, C₁₋₆acyloxy, hydroxy, and halogen or any combinations thereof. Alternatively the substituent can be C₁₋₆alkylaryl.

R² is preferably a group that contains a solubilising moiety, suitable solubilising moieties will be apparent to those skilled in the art and include basic groups. Particular solubilising groups that can be mentioned include amine and hydroxy groups. For example, amino, mono-or di-C₁₋₆alkylamino, amine containing heterocyclyl or hydroxy groups or any combination thereof.

Specific R² groups that may be mentioned include —CR⁷R⁸—CH₂-Z, —CH₂-Z and heterocyclyl, wherein R⁷ and R⁸ independently represent optionally substituted C₁₋₆alkyl, or R⁷ and R⁸ together with the carbon atom to which they are attached form an optionally substituted C₃₋₇cycloalkyl or C₅₋₇cycloalkenyl ring; and Z is NR⁹R¹⁰, NR²C(O)NR⁹R¹⁰, NR⁹COOR¹⁰, NR⁹SO₂R¹⁰, NR⁹C(O)R¹⁰ or heterocyclyl wherein R⁹ and R¹⁰ are independently selected from hydrogen, C₁₋₆alkyl, C₃₋₇cycloalkyl, heterocyclyl, heterocyclylC₁₋₆alkyl, aryl, arylC₁₋₆alkyl, heteroaryl and heteroarylC₁₋₆alkyl, any of which may be optionally substituted or together form a heterocyclic group, when present as NR⁹R¹⁰; Q is O or S, preferably O; and when R² or Z is heterocyclyl, e.g. piperidyl, piperazine or morpholine, the heterocyclyl group is optionally substituted.

Specific R² groups that may be mentioned include optionally substituted phenyl, pyridyl, pyrimidyl and furanyl.

Further specific R² groups which may be mentioned included phenyl substituted by a group —O—(CH₂)ₘ—NR¹⁸R¹⁹ or —(CH₂)ₘ—NR¹⁸R¹⁹, wherein m is an integer from 1 to 6, e.g. 2 or 3, and R¹⁸ and R¹⁹ independently represent hydrogen, C₁₋₆alkyl, or R¹⁸ and R¹⁹ together with the nitrogen atom to which they are attached form an optionally substituted 5- to 7-membered ring optionally containing an additional heteroatom selected from NR²⁰ and 0, wherein R²⁰ is hydrogen or C₁₋₆alkyl, e.g. morpholinyl.

Alternatively R¹ or R⁸ can be hydrogen.

R³ is preferably hydrogen.

R⁴ is preferably hydrogen.

R⁶ is preferably hydrogen.

A group of preferred species of compounds of formula (V) includes the group:
5-[2-(2-amino-1,1-dimethyl-ethyl)-5-pyridin-4-yl-1H-imidazol-4-yl]-indan-1-one oxime 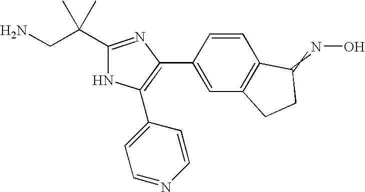
N-{2-[5-(1-hydroxyimino-indan-5-yl)-4-pyridin-4-yl-1H-imidazol-2-yl]-2-methyl-propyl}-methanesulfonamide 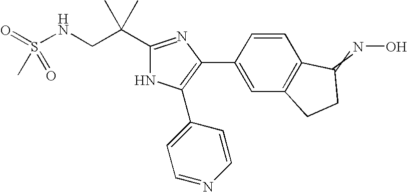
1-(2-methoxy-ethyl)-piperidine-4-carboxylic acid {2-[5-(1-hydroxyimino-indan-5-yl)-4-pyridin-4-yl-7H-imidazol-2-yl]-2-methyl-propyl}-amide 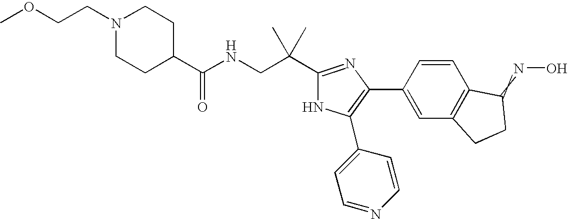
5-(2-piperidin-4-yl-5-pyridin-4-yl-1H-imidazol-4-yl)-indan-1-one oxime 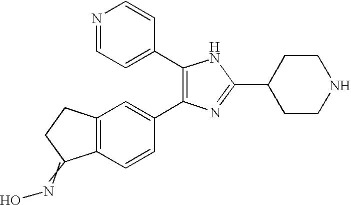
5-[2-(1-{[1-[1-(2-methoxy-ethyl)-piperidin-4-yl]-mathanoyl}-piperidin-4-yl)-5-pyridin-4-yl-1H-imidazol-4-yl]-indan-1-one oxime 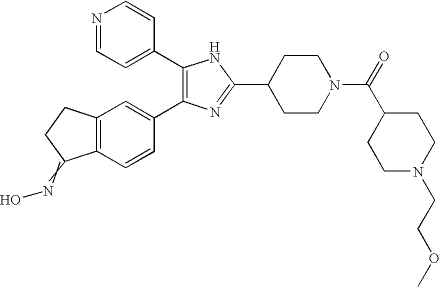
5-[2-(1-furan-3-ylmethyl-piperidin-4-yl)-5-pyridin-4-yl-1H-imidazol-4-yl]-indan-1-one oxime 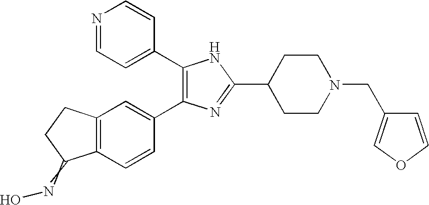
5-{2-[1-(2-methoxy-ethyl)-piperidin-4-yl]-5-pyridin-4-yl-1H-imidazol-4-yl}-indan-1-one oxime 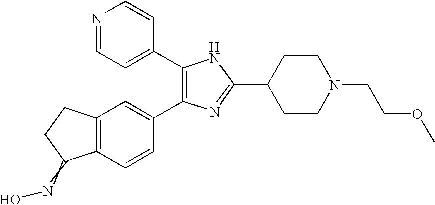
5-(2-aminomethyl-5-pyridin-4-yl-1H-imidazol-4-yl)-indan-1-one oxime 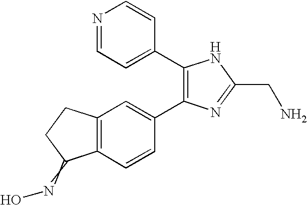
1-(2-methoxy-ethyl)-piperidine-4-carboxylic acid [4-(1-hydroxyimino-indan-5-yl)-5-pyridin-4-yl)-1H-imidazol-2-ylmethyl]-amide 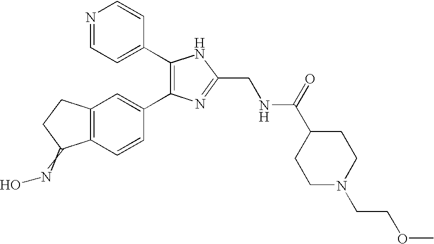
5-(2-piperidin-1-ylmethyl-5-pyridin-4-yl-1H-imidazol-4-yl)-indan-1-one oxime 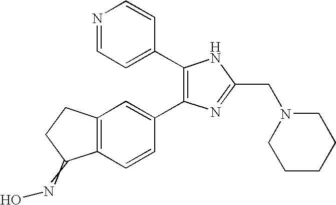
5-(2-morpholin-4-ylmethyl-5-pyridin-4-yl-1H-imidazol-4-yl)-indan-1-one oxime 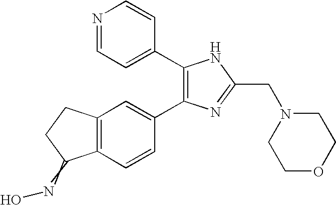
5-(5-pyridin-4-yl-2-(2,3,5,6-tetrahydro-[1,2′]bipyrazin-4-ylmethyl)-1H-imidazol-4-yl]-indan-1-one oxime 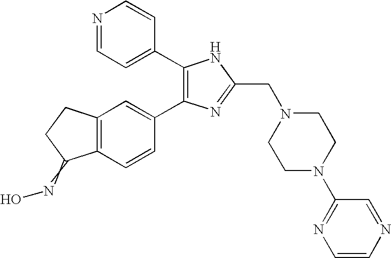
5-(2-piperazin-1-ylmethyl-5-pyridin-4-yl-1H-imidazol-4-yl]-indan-1-one oxime 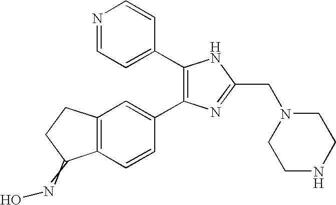
5-{2-[4-(3-dimethylamino-propyloxy)-phenyl]-5-pyridin-4-yl-1H-imidazol-4-yl}-indan-1-one 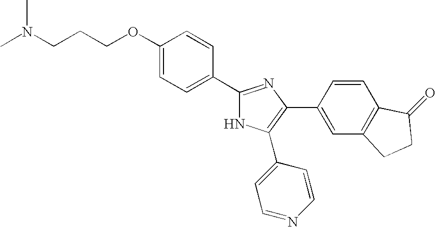
5-{2-[4-(3-dimethylamino-propyloxy)-phenyl]-5-pyridin-4-yl-1H-imidazol-4-yl}-indan-1-one oxime 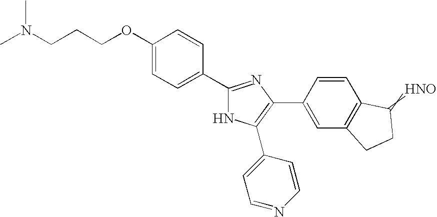
5-{2-[4-(2-dimethylamino-ethoxy)-phenyl]-5-pyridin-4-yl-1H-imidazol-4-yl}-1-indanone 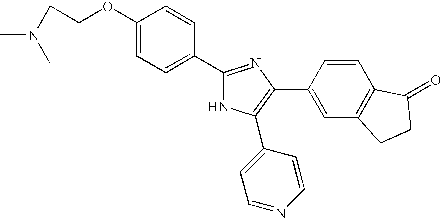
5-{2-[4-(2-dimethylamino-ethoxy)-phenyl]-5-pyridin-4-yl-1H-imidazol-4-yl}-indan-1-one oxime 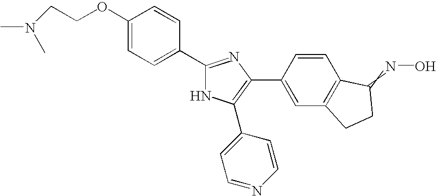
5-{2-[4-(2-morpholin-4-yl-ethoxy)-phenyl]-5-pyridin-4-yl-1H-imidazol-4-yl}-indan-1-one 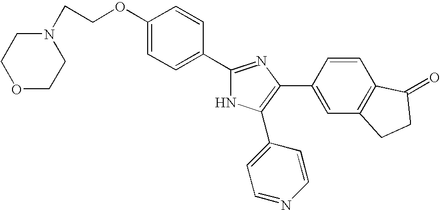
5-{2-[4-(2-morpholin-4-yl-ethoxy)-phenyl]-5-pyridin-4-yl-1H-imidazol-4-yl}-indan-1-one oxime 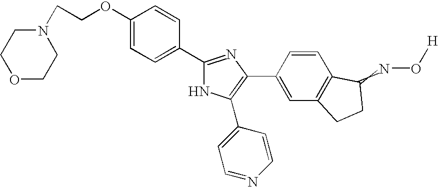
5-(5-pyridin-4-yl-2-pyridin-3-yl-1H-imidazol-4-yl)-indan-1-one 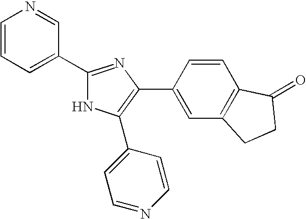
5-(5-pyridin-4-yl-2-pyridin-3-yl-1H-imidazol-4-yl}-indan-1-one oxime 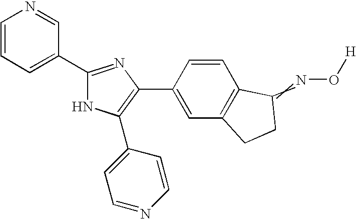
5-(2-phenyl-5-pyridin-4-yl-1H-imidazol-4-yl)-indan-1-one 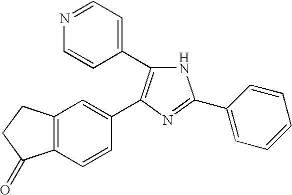
5-(2-phenyl-5-pyridin-4-yl-1H-imidazol-4-yl)-indan-1-one oxime

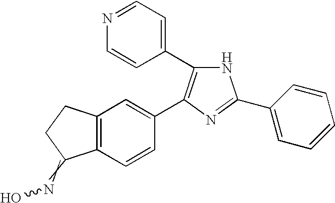

These compounds and other compounds of Formula (V) may be prepared according to the procedures of International Patent Application WO 02/24680, filed Sep. 19, 2001, and published on Mar. 28, 2002.

Another class of bRaf inhibitor compounds that may be usefully employed in the present invention includes compounds of the Formula VI: 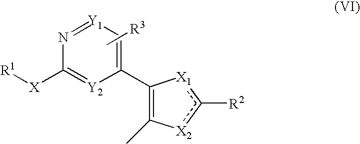
wherein
X is O, CH₂, CO, S or NH, or the moiety X—R¹ is hydrogen;
Y₁ and Y₂ independently represent CH or N;
R¹ is hydrogen, C₁₋₆alkyl, C₃₋₇cycloalkyl, aryl, arylC₁₋₆alkyl-, heterocyclyl, heterocyclylC₁₋₆alkyl-, heteroaryl, or heteroarylC₁₋₆alkyl-, any of which, except hydrogen, may be optionally substituted;
R² is C₁₋₆alkyl, C₃₋₇cycloalkyl, heterocyclyl, heterocyclylC₁₋₆alkyl-, heteroC₁₋₆alkyl-, or C₁₋₆alkylheteroC₁₋₆alkyl, any of which may be optionally substituted;
Ar is a group of the formula a) or b): 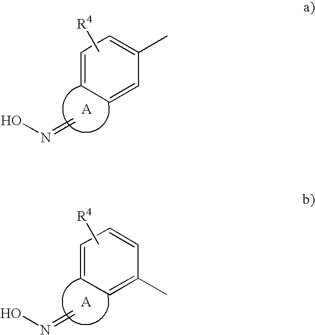
wherein A represents a fused 5- to 7-membered ring optionally containing up to two heteroatoms selected from O, S and NR⁵, wherein R⁵ is hydrogen or C₁₋₆alkyl, which ring is optionally substituted by up to 2 substituents selected from halogen, C₁₋₆alkyl, hydroxy, C₁₋₆alkoxy or keto;
R³ and R⁴ are independently selected from hydrogen, halogen, C₁₋₆alkyl, aryl, arylC₁₋₆alkyl, C₁₋₆alkoxy, C₁₋₆alkoxyC₁₋₆alkyl, haloC₁₋₆alkyl, arylC₁₋₆alkoxy, hydroxy, nitro, cyano, azido, amino, mono- and di-N-C₁₋₆alkylamino, acylamino, arylcarbonylamino, acyloxy, carboxy, carboxy salts, carboxy esters, carbamoyl, mono- and di-N-C₁₋₆alkylcarbamoyl, C₁₋₆alkoxycarbonyl, aryloxycarbonyl, ureido, guanidino, C₁₋₆alkylguanidino, amidino, C₁₋₆alkylamidino, sulphonylamino, aminosulphonyl, C₁₋₆alkylthio, C₁₋₆alkylsulphinyl or C₁₋₆alkylsulphonyl; and
one of X₁ and X₂ is selected from O, S or NR¹¹ and the other is CH, wherein R¹¹ is hydrogen, C₁₋₆alkyl, aryl or arylC₁₋₆alkyl;
or pharmaceutically acceptable salts thereof.

As used herein, the double bond indicated by the dotted lines of formula (I), represents the possible regioisomeric ring forms of the compounds falling within the scope of this invention, the double bond being between the non-hetero-atoms.

The hydroxyimino moiety can be positioned on any of carbon atoms of the non-aromatic ring in groups a) and b).

The hydroxyimino moiety can exist as either the E or Z isomer or as a mixture of both.

The alkyl, alkenyl, cycloalkyl and cycloalkenyl groups are optionally subastituted as defined above. Alternatively, the optional substituent contains a water-solubilising group; suitable solubilising moieties will be apparent to those skilled in the art and include hydroxy and amine groups. Even more preferably the optional substituent includes amino, mono- or di-C₁₋₆alkylamino, amine containing heterocyclyl, or hydroxy or any combination thereof.

When used herein heteroC₁₋₆alkyl- means a C₁₋₆ carbon chain wherein the end carbon atom in the chain is substituted by a heteroatom selected from N, O, or S for example C₁₋₆alkylamino, C₁₋₆alkyloxy or C₁₋₆alkylthio.

C₁₋₆alkylheteroC₁₋₆alkyl means a C₃₋₁₃alkyl chain wherein one of the carbon atoms has been replaced with a heteroatom selected from N, O, or S, for example C₁₋₆alkylaminoC₁₋₆alkyl or C₁₋₆ alkylaminodiC₁₋₆alkyl, C₁₋₆alkyloxyC₁₋₆alkyl-, C₁₋₆alkylthioC₁₋₆alkyl-, or C₁₋₆ alkylthiodiC₁₋₆alkyl.

Aryl, heterocyclyl and heteroaryl groups may be optionally substituted as defined above. Preferably the optional substituent contains a water-solubilising group; suitable solubilising moieties will be apparent to those skilled in the art and include hydroxy and amine groups. Even more preferably the optional substituent includes amino, mono- or di-C₁₋₆alkylamino, amine containing heterocyclyl, or hydroxy or any combination thereof.

X is preferably NH or X—R¹ is preferably hydrogen and when X is NH, R¹ is preferably hydrogen or C₁₋₆alkyl.

When Y₁ and Y₂ are CH, X—R¹ is preferably hydrogen.

When Y₂ is N, R¹ is preferably H or C₁₋₆alkyl.

Most preferably X—R¹ is hydrogen.

Preferably X₁ or X₂ is S or O, more preferably 0.

Preferably R¹¹ is hydrogen.

A is preferably a fused 5 membered ring optionally containing up to two heteroatoms selected from O, S and NR⁵, wherein R⁵ is hydrogen or C₁₋₆alkyl, which ring is optionally substituted by up to 2 substituents selected from halogen, C₁₋₆alkyl, hydroxy, C₁₋₆alkoxy or keto.

Even more preferably A is a fused 5 membered ring.

Preferably R² is an optionally substituted heterocyclyl, heterocyclyl(C₁₋₆)alkyl- or C₁₋₆alkylheteroC₁₋₆alkyl.

Most preferably the compounds of the invention are of formula (II); 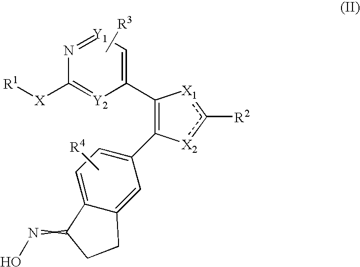
wherein R¹, X, Y₁, Y₂, R³, X₁, X₂, R² and R⁴ are as described for compounds of formula (I).

Preferred substituents for the group Ar include halo, hydroxy, hydroxyC₁₋₆alkyl, hydroxyimino-C₁₋₆alkyl and C₁₋₆alkoxy.

It will be understood that the invention includes pharmaceutically acceptable derivatives of compounds of formula (VI) and that these are included within the scope of the invention.

Particular compounds according to the invention include those mentioned in the examples and their pharmaceutically acceptable salts. As used herein “pharmaceutically acceptable derivatives” includes any pharmaceutically acceptable salt, ester or salt of such ester of a compound of formula (VI), which upon administration to the recipient, is capable of providing (directly or indirectly) a compound of formula (I) or an active metabolite or residue thereof.

A group of preferred species of compounds of formula (VI) includes the group:
5-(5-morpholin-4-ylmethyl-2-pyridin-4-yl-furan-3-yl)-indan-1-one oxime 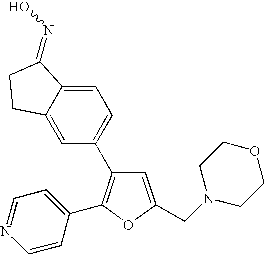
5-(5-piperidin-1-ylmethyl-2-pyridin-4-yl-furan-3-yl)-indan-1-one oxime;
5-(2-pyridin-4-yl-5-pyrrolidin-1-ylmethyl-furan-3-yl)-indan-1-one oxime;
5-{5-(4-methyl-piperazin-1-ylmethyl)-2-pyridin-4-yl-furan-3-yl]-indan-1-one oxime;
5-[5-(1,1-dioxo-1-thiomorpholin-4-ylmethyl)-2-pyridin-4-yl-furan-3-yl]-indan-1-one oxime;
5-(5-piperazin-1-ylmethyl-2-pyridin-4-yl-furan-3-yl)-indan-1-one oxime;
5-(5-dimethylaminomethyl-2-pyridin-4-yl-furan-3-yl)-indan-1-one oxime;
5-{5-[(2-methoxyethylamino)-methyl]-2-pyridin-4-yl-furan-3-yl}-indan-1-one oxime;
5-(5-{[1-(2-methoxy-ethyl)-piperidin-4-ylamino]-methyl}-3-pyridin-4-yl-furan-2-yl)-indan-1-one oxime 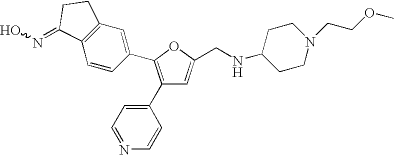
5-(5-morpholin-4-ylmethyl-3-pyridin-4-yl-furan-2-yl)-indan-1-one oxime;
5-(5-piperidin-1-ylmethyl-3-pyridin-4-yl-furan-2-yl)-indan-1-one oxime;
5-[3-pyridin-4-yl-5-(4-pyrrolidin-1-yl-piperidin-1-ylmethyl)-furan-2-yl]-indan-1-one oxime;
5-{5-[(2-methoxy-ethylamino)-methyl]-3-pyridin-4-yl-furan-2-yl}-indan-1-one oxime;
5-(5-diethylaminomethyl-3-pyridin-4-yl-furan-2-yl)-indan-1-one oxime;
5-[5-(4-ethyl-piperazin-1-ylmethyl)-3-pyridin-4-yl-furan-2-yl]-indan-1-one oxime;
5-{5-[4-(2-methoxy-ethyl)-piperazin-1-ylmethyl]-3-pyridin-4-yl-furan-2-yl}-indan-1-one oxime;
5-{5-[(2-morpholin-4-yl-ethylamino)-methyl]-3-pyridin-4-yl-furan-2-yl}-indan-1-one oxime;
5-(5-{[methyl-(1-methyl-piperidin-4-yl)-amino]-methyl}-3-pyridin-4-yl-furan-2-yl)-indan-1-one oxime;
5-[5-(4-methyl-piperazin-1-ylmethyl)-3-pyridin-4-yl-furan-2-yl]-indan-1-one oxime;
5-(3-pyridin-4-yl-5-pyrrolidin-1-ylmethyl-furan-2-yl)-indan-1-one oxime;
5-(5-dimethylaminomethyl-3-pyridin-4-yl-furan-2-yl)-indan-1-one oxime;
5-{5-[4-(2-hydroxy-ethyl)-piperazin-1-ylmethyl]-3-pyridin-4-yl-furan-2-yl}-indan-1-one oxime;
5-(5-{[(2-methoxy-ethyl)-methyl-amino]-methyl}-3-pyridin-4-yl-furan-2-yl)-indan-1-one oxime;
5-(5-{[isopropyl-(2-methoxy-ethyl)-amino]-methyl}-3-pyridin-4-yl-furan-2-yl)-indan-1-one oxime;
5-[5-(1,1-dioxo-1-thiomorpholin-4-ylmethyl)-3-pyridin-4-yl-furan-2-yl]-indan-1-one oxime;
5-(5-piperazin-1-ylmethyl-3-pyridin-4-yl-furan-2-yl)-indan-1-one oxime;
5-(5-piperidin-1-ylmethyl-2-pyrimidin-4-yl-furan-3-yl)-indan-1-one oxime 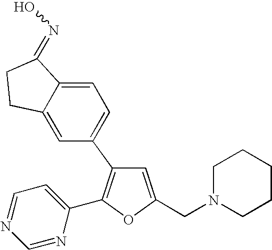
5-[2-(2-amino-pyrimidin-4-yl)-5-piperidin-1-ylmethyl-furan-3-yl]-indan-1-one oxime 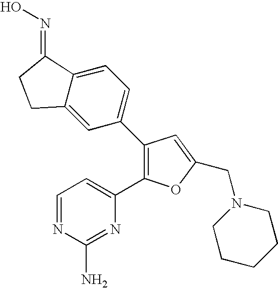
5-[5-(4-hydroxy-piperidin-4-yl)-2-pyridin-4-yl-furan-3-yl]-indan-1-one oxime 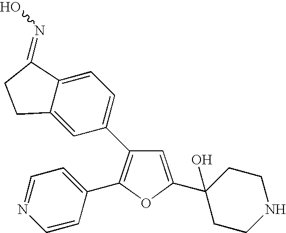
5-[2-pyridin-4-yl-5-(1,2,3,6-tetrahydro-pyridin-4-yl)-furan-3-yl]-indan-1-one oxime 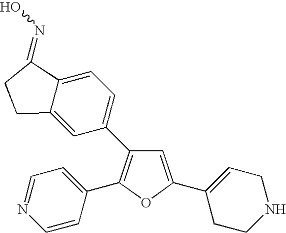
5-(5-piperidin-4-yl-2-pyridin-4-yl-furan-3-yl)-indan-1-one oxime 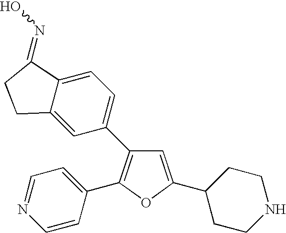
5-{5-[1-(2-methoxyethyl)-piperidin-4-yl]-2-pyridin-4-yl-furan-3-yl}-indan-1-one oxime 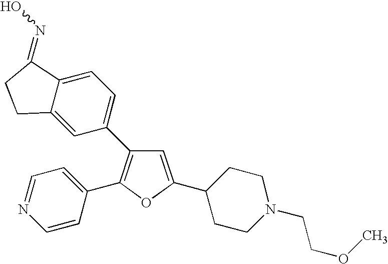
5-(5-{1-[2-(4-chloro-phenoxy)-ethyl]-piperidin-4-yl}-2-pyridin-4-yl-furan-3-yl)-indan-1-one oxime 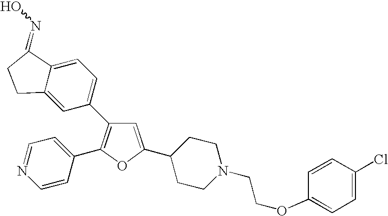
5-[5-(1-cyclopentyl-piperidin-4-yl)-2-pyridin-4-yl-furan-3-yl]-indan-1-one oxime;
5-[5-(1-cyclopropylmethyl-piperidin-4-yl)-2-pyridin-4-yl-furan-3-yl]-indan-1-one oxime;
5-{5-[1-(2-morpholin-4-yl-ethyl)-piperidin-4-yl]-2-pyridin-4-yl-furan-3-yl}-indan-1-one oxime;
5-[5-(1-methanesulfonyl-piperidin-4-yl)-2-pyridin-4-yl-furan-3-yl]-indan-1-one oxime 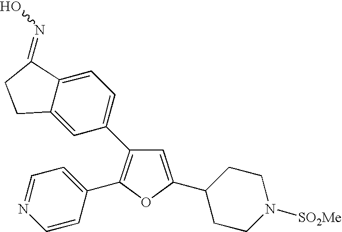
5-{5-[1-(2-dimethylamino-ethanoyl)-piperidin-4-yl]-2-pyridin-4-yl-furan-3-yl}-indan-1-one oxime 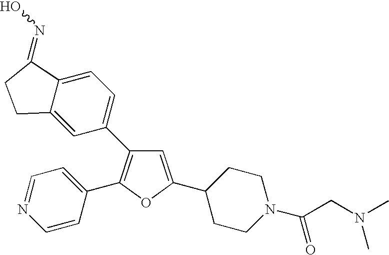
5-{5-[1-(3-piperidin-1-yl-propanoyl)-piperidin-4-yl]-2-pyridin-4-yl-furan-3-yl}-indan-1-one oxime;
5-(5-piperidin-4-yl-3-pyridin-4-yl-furan-2-yl)-indan-1-one oxime 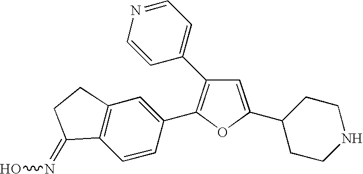
5-{5-[1-(2-methoxyethyl)piperidin-4-yl]-3-pyridin-4-ylfuran-2-yl}indan-1-one oxime 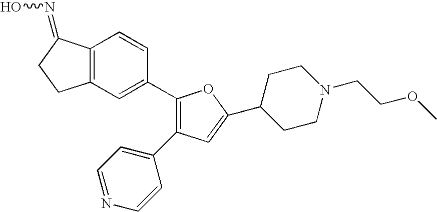
5-[5-(1-cyclopropylmethylpiperidin-4-yl)-3-pyridin-4-ylfuran-2-yl]indan-1-one oxime;
5-[5-(1-cyclopentylpiperidin-4-yl)-3-pyridin-4-ylfuran-2-yl]indan-1-one oxime;
5-(5-{1-[2-(4-chlorophenoxy)ethyl]-piperidin-4-yl}-3-pyridin-4-ylfuran-2-yl)indan-1-one oxime;
{4-[5-(1-hydroxyiminoindan-5-yl)-4-pyridin-4-ylfuran-2-yl]piperidin-1-yl}acetonitrile 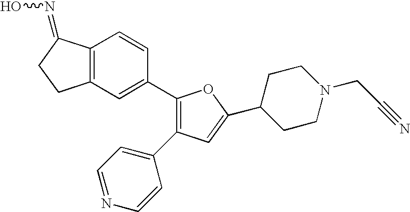
5-{5-[1-(2-hydroxyethyl)piperidin-4-yl]-3-pyridin-4-ylfuran-2-yl}indan-1-one oxime 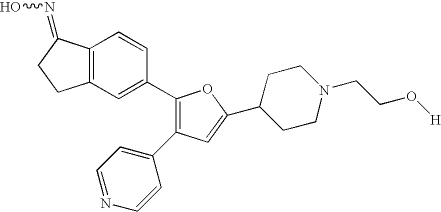
5-{5-[1-(2-morpholin-4-ylethanoyl)piperidin-4-yl]-3-pyridin-4-ylfuran-2-yl}indan-1-one oxime 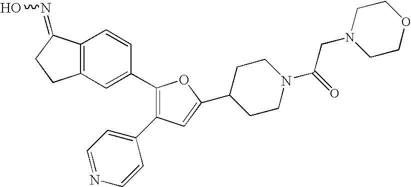
5-{5-[1-(2-piperidin-1-ylethanoyl)piperidin-4-yl]-3-pyridin-4-ylfuran-2-yl}indan-1-one oxime 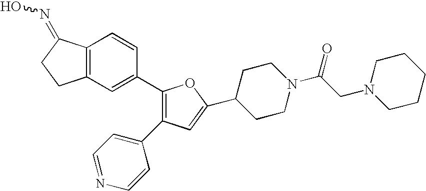
5-{5-[1-(2-piperazin-1-ylethanoyl)piperidin-4-yl]-3-pyridin-4-ylfuran-2-yl}indan-1-one oxime 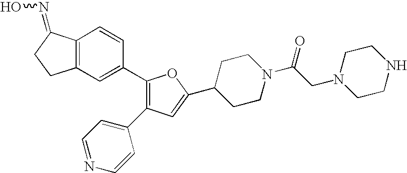
5-(5-piperidin-3-yl-2-pyridin-4-yl-furan-3-yl)-indan-1-one oxime 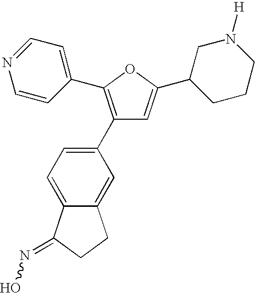
5-[5-(1-methyl-piperidin-3-yl)-2-pyridin-4-yl-furan-3-yl]-indan-1-one oxime 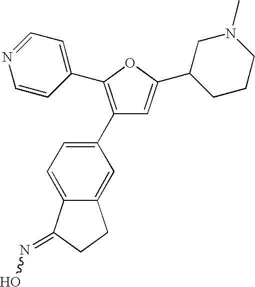
N-hydroxy-2-{3-[4-(1-hydroxyimino-indan-5-yl)-5-pyridin-4-yl-furan-2-yl]-piperidin-1-yl}-acetamidine 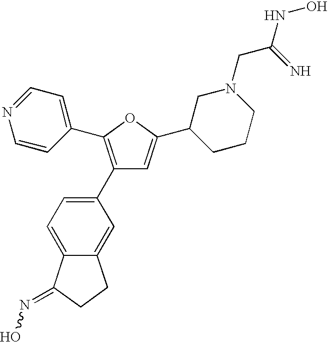
5-{5-[1-(2-methoxy-ethyl)-piperidin-3-yl]-2-pyridin-4-yl-furan-3-yl}-indan-1-one oxime 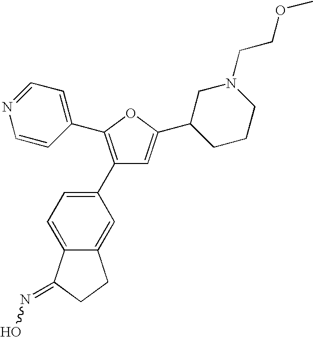
5-[5-(1-cyclopropylmethyl-piperidin-3-yl)-2-pyridin-4-yl-furan-3-yl]-indan-1-one oxime 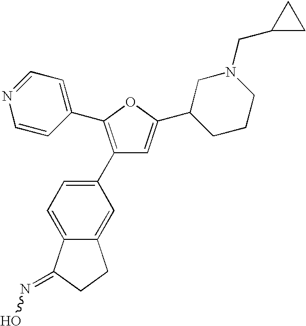
5-{5-[1-(2-morpholin-4-yl-ethanoyl)-piperidin-3-yl]-2-pyridin-4-yl-furan-3-yl)-indan-1-one oxime 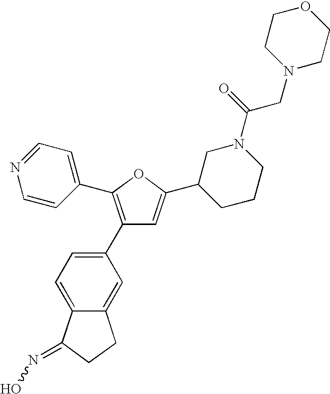
5-{5-[1-(2-piperidin-1-yl-ethanoyl)-piperidin-3-yl]-2-pyridin-4-yl-furan-3-yl}-indan-1-one oxime;
5-{2-pyridin-4-yl-5-[1-(2-pyrrolidin-1-yl-ethanoyl)-piperidin-3-yl]-furan-3-yl}-indan-1-one oxime;
5-(5-{1-[2-(4-methyl-piperazin-1-yl)-ethanoyl]-piperidin-3-yl}-2-pyridin-4-yl-furan-3-yl)-indan-1-one oxime;
5-[5-(1-{2-[4-(2-methoxy-ethyl)-piperazin-1-yl]-ethanoyl}-piperidin-3-yl)-2-pyridin-4-yl-furan-3-yl]-indan-1-one oxime;
5-{5-[4-hydroxy-1-(2-methoxyethyl)-piperidin-4-yl]-2-pyridin-4-yl-furan-3-yl}-indan-1-one oxime;
5-[5-(1-cyclopropyl methyl-4-hydroxy-piperidin-4-yl)-2-pyridin-4-yl-furan-3-yl]-indan-1-one oxime;
5-(5-hydroxymethyl-2-pyridin-4-yl-furan-3-yl)indan-1-one oxime

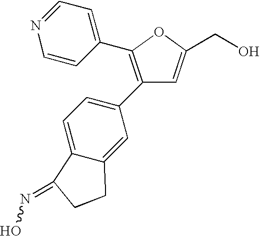

These compounds and other compounds of Formula (VI) may be prepared according to the procedures of International Patent Application WO 03/022840, filed Sep. 5, 2002, and published on Mar. 20, 2003.

Another group of compounds useful in the present invention are inhibitors of The Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7 (4) 292-8; Ashby, M. N. (1998), Current Opinion in Lipidology. 9 (2) 99-102; and BioChim. Biophys. Acta, (19899) 1423 (3):19-30.

The erb family inhibitor, e.g., dual EGFR/erbB-2 inhibitor and the Raf and/or ras inhibitor, e.g., cRaf-1 or bRaf inhibitor, may be employed in combination in accordance with the invention by administration concomitantly in (1) a unitary pharmaceutical composition including both compounds or (2) separate pharmaceutical compositions each including one of the compounds. Alternatively, the combination may be administered separately in a sequential manner wherein, for example, the cRaf-1 or bRaf inhibitor or dual EGFR/erbB-2 inhibitor is administered first and the other second. Such sequential administration may be close in time or remote in time.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in a compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, poiygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

While it is possible that, for use in therapy, therapeutically effective amounts of a dual EGFR/erbB2, cRaf-1 or bRaf inhbitor, as well as salts, solvates and physiological functional derivatives thereof, may be administered as the raw chemical, it is possible to present the active ingredient as a pharmaceutical composition. Accordingly, the invention further provides pharmaceutical compositions, which include therapeutically effective amounts of a dual EGFR/erbB2 and/or cRaf-1 or bRaf inhibitor and salts, solvates and physiological functional derivatives thereof, and one or more pharmaceutically acceptable carriers, diluents, or excipients. The compounds of the present invention and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a dual EGFR/erbB2 and/or a cRaf-1 or bRaf inhibitor or salts, solvates and physiological functional derivatives thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of an EGFR/erbB2 and/or cRaf-1 or bRaf inhibitor, depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

The dual EGFR/erbB-2 inhibitors and cRaf-1 or bRaf inhibitors may be administered by any appropriate route. Suitable routes include oral, rectal, nasal, topical (including buccal and sublingual), vaginal, and parenteral (including subcutaneous, intramuscular, intraveneous, intradermal, intrathecal, and epidural). It will be appreciated that the preferred route may vary with, for example, the condition of the recipient of the combination. It will also be appreciated that each of the agents administered may be administered by the same or different routes and that the erbB-2 and cRaf-1 or bRaf inhibitors may be compounded together in a pharmaceutical composition/formulation.

The method of the present invention may also be employed with other therapeutic methods of cancer treatment. In particular, in anti-neoplastic therapy, combination therapy with other chemotherapeutic, hormonal, antibody agents as well as surgical and/or radiation treatments other than those mentioned above are envisaged. Anti-neoplastic therapies are described for instance in International Application No. PCT US 02/01130, filed Jan. 14, 2002, which application is incorporated by reference to the extent that it discloses anti-neoplastic therapies. Combination therapies according to the present invention thus include the administration of at least one erbB-2 inhibitor and at least one cRaf-1 and/or bRaf inhibitor as well as optional use of other therapeutic agents including other anti-neoplastic agents. Such combination of agents may be administered together or separately and, when administered separately this may occur simultaneously or sequentially in any order, both close and remote in time. The amounts of the erbB2, cRaf-1, and bRaf inhibitors and the other pharmaceutically active agent(s) and the relative timings of administration will be selected in order to achieve the desired combined therapeutic effect.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a table or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The agents for use according to the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Agents for use according to the present invention may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3 (6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container or the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists that may be generated by means of various types of metered dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavoring agents.

Also, contemplated in the present invention is a pharmaceutical combination including at least one erb family inhibitor, such as a dual erbB-2/EGFR inhibitor and at least one Raf and/or ras inhibitor such as a cRaf-1 inhibitor or bRaf inhibitor. In another embodiment, the pharmaceutical combination includes an erbB-2 inhibitor, a cRaf-1 inhibitor or bRaf inhibitor, and optionally at least one additional anti-neoplastic agent. The erb inhibitors, raf and ras inhibitors, and additional anti-neoplastic therapy are as described above.

As indicated, therapeutically effective amounts of the specific erb family inhibitor and Raf and/or ras inhibitor are administered to a mammal. Typically, the therapeutically effective amount of one of the administered agents of the present invention will depend upon a number of factors including, for example, the age and weight of the mammal, the precise condition requiring treatment, the severity of the condition, the nature of the formulation, and the route of administration. Ultimately, the therapeutically effective amount will be at the discretion of the attendant physician or veterinarian.

Typically, the erb family and Raf and/or ras inhibitors will be given in the range of 0.1 to 100 mg/kg body weight of recipient (mammal) per day and more usually in the range of 1 to 10 mg/kg body weight per day.

The following examples are intended for illustration only and are not intended to limit the scope of the invention in any way.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| N (Normal) | Kg (kilogram) |
| i. v. (intravenous); | Hz (Hertz); |
| MHz (megahertz); | mol (moles); |
| mmol (millimoles); | RT (room temperature); |
| min (minutes); | h (hours); |
| mp (melting point); | TLC (thin layer chromatography); |
| Tᵣ (retention time); | RP (reverse phase); |
| DCM (dichloromethane); | DCE (dichloroethane); |
| DMF (N,N-dimethylformamide); | HOAc (acetic acid); |
| TMSE (2-(trimethylsilyl)ethyl); | TMS (trimethylsilyl); |
| TIPS (triisopropylsilyl); | TBS (t-butyldimethylsilyl); |
| HPLC (high pressure liquid | |
| chromatography); | |
| THF (tetrahydrofuran); | DMSO (dimethylsulfoxide); |
| EtOAc (ethyl acetate); | DME (1,2-dimethoxyethane); |
| EDTA | ethylenediaminetetraacetic acid |
| FBS | fetal bovine serum |
| IMDM | Iscove's Modified Dulbecco's medium |
| PBS | phosphate buffered saline |
| RPMI | Roswell Park Memorial Institute |
| RIPA buffer | * |
| RT | room temperature |

* 150 mM NaCl, 50 mM Tris-HCl, pH 7.5, 0.25% (w/v) deoxycholate, 1% NP-40, 5 mM sodium orthovanadate, 2 mM sodium fluoride, and a protease inhibitor cocktail.

Unless otherwise indicated, all temperatures are expressed in ° C. (degrees Centigrade). All reactions conducted under an inert atmosphere at room temperature unless otherwise noted.

¹H NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX505HA, JOEL SX-102, or a SCIEX-APliii spectrometer; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck). Optical rotations were obtained using a Perkin Elmer Model 241 Polarimeter. Melting points were determined using a Mel-Temp II apparatus and are uncorrected.

Examples 1-5 recite the preparation of specific erbB-2/EGFR inhibitors useful in the present invention.

### Example 1

### Monohydrate Ditosylate Salt of N-{3-Chloro-4-[(3-fluorobenzyl)oxy]phenyl}-6-[5-({[2-(methanesulphonyl)ethyl]amino}methyl)-2-furyl]-4-quinazolinamine (Monohydrate Ditosylate Salt of Compound of Formula (III))

### 1(a) Preparation of N-{3-Chloro-4-[(3-fluorobenzyl)oxy]phenyl}-6-[5-({[2-(methanesulphonyl)ethyl]amino}methyl)-2-furyl]-4-quinazolinamine (Free Base of Compound of Formula (III))

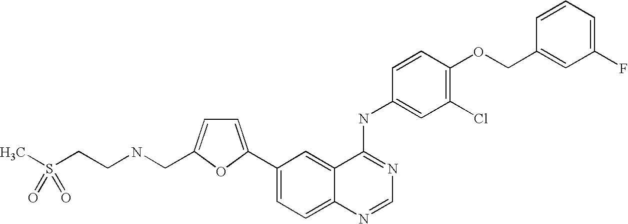

The title compound was prepared according to Procedure D of International Applications WO 02/02552: p. 16, line 19 to p. 17, line 3 and WO 99/35146: p. 56, lines 20-32 and Example 29 p. 100, lines 18-29, from 5-(4-{3-chloro-4-(3-fluorobenzyloxy)-anilino}-6-quinazolinyl)-furan-2-carbaldehyde (0.6 equiv) and 2-methanesulphonyl-ethylamine (1 equiv). ¹H NMR 400 MHz (DMSO-d₆) 9.60 (bs, 1H); 9.32 (bs, 1H); 8.82 (bs, 1H); 8.34 (d, 1H); 8.0 (s, 1H); 7.88 (d. 1H); 7.74 (d, 1H); 7.45 (m, 1H); 7.34-7.23 (m, 4H); 7.17 (m, 1H); 6.83 (d, 1H); 5.27 (s, 2H); 4.42 (s, 2H); 3.59 (m, 2H); 3.40 (m, 2H, obscured by waterpeak); 3.12 (s, 3H); MS m/z 581 (M+H⁺).

### 1(b) Preparation of Monohydrate Ditosylate Salt of N-{3-Chloro-4-[(3-fluorobenzyl)oxy]phenyl}-6-[5-({[2-(methanesulphonyl)ethyl]amino}methyl)-2-furyl]-4-quinazolinamine (Monohydrate Ditosylote Salt of Compound of Formula (III))

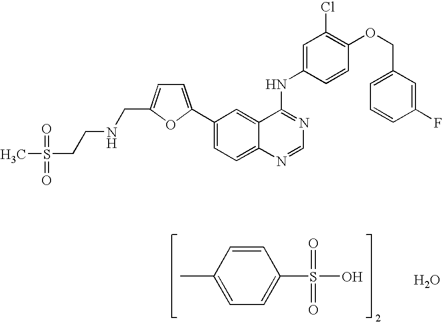

### Stage 1: Preparation of N-{3-chloro-4-[(3-fluorobenzyl)oxy]phenyl}-6-iodo-4-quinazolinamine

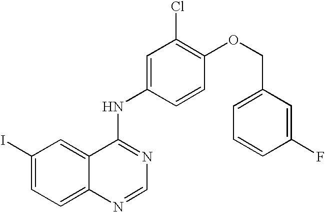

4-Chloro-6-iodoquinazoline (1 wt) was added to a solution of fluorobenzyloxyaniline (0.894 wt, 1.03 equiv) in N-methylpyrrolidinone (8.26 wt, 8 vol) at ca 20° C., and after the initial exotherm had subsided, the resulting solution was stirred at 20°-25° C. for at least 30 minutes. The dark solution was treated with triethylamine (0.58 vol, 1.2 equiv) and the mixture was stirred for 20-30 minutes. Isopropanol (2.5 vol) was added and the mixture was heated to ca 50° C. Water (up to 3 vol) was added slowly to the vessel over 10-15 minutes, while keeping the temperature at ca 50° C. Once crystallisation had commenced the addition was stopped and the resulting slurry was aged for 30-45 minutes at ca 50° C. Any residual water (from the 3 vol) was added, then further water (5 vol) was added to the vessel over ca 30 minutes while maintaining the temperature at ca 50° C. The resulting slurry was cooled to ca 20° C. over ca 30 minutes and aged at ca 20° C. for at least 30 minutes. The solid was collected by filtration and washed sequentially with water (2×5 vol), then isopropanol (5 vol). The product was dried in vacuo at ca 60° C. to give the title compound as a cream crystalline solid.

### Stage 2: Preparation of 5-(4-[3-chloro-4-(3-fluorobenzyloxy)-anilino]-6-quinazolinyl)-furan-2-carbaldehyde 4-methylbenzenesulfonate

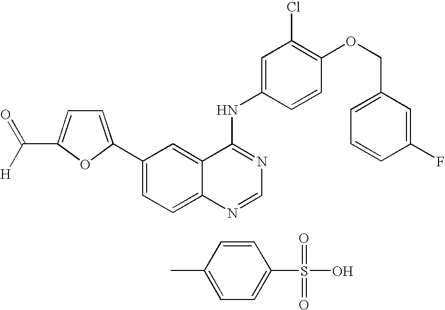

A mixture of N-{3-chloro-4-[(3-fluorobenzyl)oxy]phenyl}-6-iodo-4-quinazolinamine (1 wt), boronic acid (0.37 wt, 1.35 equiv), and 10% palladium on charcoal (0.028 wt, 50% water wet) was slurried in IMS (15 vol). The resultant suspension was stirred for 5 minutes, treated with di-isopropylethylamine (0.39 vol, 1.15 equiv) and then heated to ca 70° C. for ca 3 hours when the reaction was complete (determined by HPLC analysis). The mixture was diluted with tetrahydrofuran (THF, 15 vol) and then filtered (hot—through GFA filter paper) to remove catalyst. The vessel was rinsed with IMS (2 vol).

A solution of p-toluenesulfonic acid monohydrate (1.54 wt, 4.1 equiv) in water (3 vol) was added over 5-10 minutes to the filtered solution maintained at 65° C. After crystallisation the suspension was stirred at 601-65° C. for 1 hour, cooled to ca 25° C. over 1 hour and stirred at this temperature for a further 2 hours. The solid was collected by filtration, washed with IMS (3 vol) then dried in vacuo at ca 50° C. to give the tile compound as a yellow-orange crystalline solid.

### Stage 3: Preparation of Anhydrous Ditosylate Salt of N-{3-Chloro-4-[(3-fluorobenzyl)oxy]phenyl}-6-[5-({[2-(methanesulphonyl)ethyl]amino}methyl)-2-furyl]-4-quinazolinamine (Anhydrous Ditosylate Salt of Compound of Formula (III))

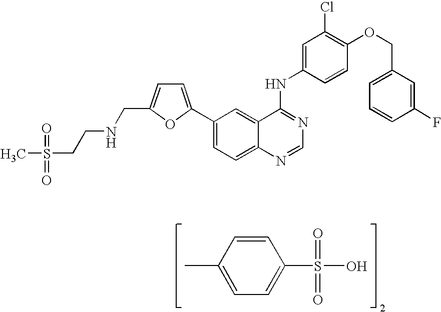

5-(4-[3-chloro-4-(3-fluorobenzyloxy)-anilino]-6-quinazolinyl)-furan-2-carbaldehyde 4-methylbenzenesulfonate (1 wt) and 2-(methylsulfonyl)ethylamine hydrochloride (0.4 wt, 1.6 equiv) were suspended in THF (10 vol). Sequentially, acetic acid (0.35 vol, 4 equiv) and di-isopropylethylamine (1.08 vol, 4 equiv) were added. The resulting solution was stirred at 30°-35° C. for ca 1 hour then cooled to ca 23° C. Sodium triacetoxyborohydride (0.66 wt, 2 equiv) was then added as a continual charge over approximately 15 minutes (some effervescence is seen at this point). The resulting mixture was stirred at ca 22° C. for ca 2 hours then sampled for HPLC analysis. The reaction was quenched by addition of 5M aqueous sodium hydroxide (5 vol) and stirred for ca 30 minutes (some effervescence is seen at the start of the caustic addition).

The aqueous phase was then separated, extracted with THF (2 vol) and the combined THF extracts were then washed with 10% w/v aqueous sodium chloride solution (4 vol). A solution of p-toluenesulfonic acid monohydrate (pTSA, 1.77 wt, 6 equiv) in THF (7 vol) was prepared and warmed to ca 55° C. The THF solution of N-{3-Chloro-4-[(3-fluorobenzyl)oxy]phenyl}-6-[5-({[2-(methanesulphonyl)ethyl]amino}methyl)-2-furyl]-4-quinazolinamine was added to the pTSA solution over at least 30 minutes, maintaining the batch temperature at ca 55° C. 2. The resulting suspension was stirred at ca 55° C. for 2 hours, cooled to 20°-25° C. over ca 60 minutes and aged at this temperature for ca 30 minutes. The solid was collected by filtration, washed with THF (2×2 vol) and dried in vacuo at ca 40° C. to give the desired compound as a pale yellow crystalline solid.

### Stage 4: Preparation of Monohydrate Ditosylote Salt of N-{3-Chloro-4-[(3-fluorobenzyl)oxy]phenyl}-6-[5-({[2-(methanesulphonyl)ethyl]amino}methyl)-2-furyl]-4-quinazolinamine (Monohydrate Ditosylate Salt of Compound of Formula (III))

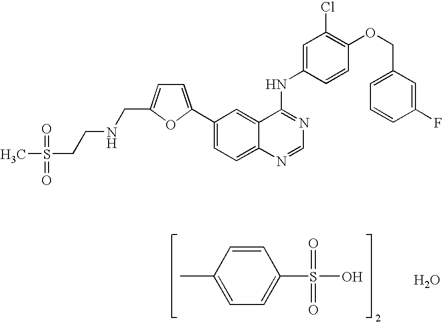

A suspension of the anhydrous ditosylate salt of N-{3-Chloro-4-[(3-fluorobenzyl)oxy]phenyl}-6-[5-({[2-(methanesulphonyl)ethyl]amino}methyl)-2-furyl]-4-quinazolinamine (1 wt), in tetrahydrofuran (THF, 14 vol) and water (6 vol) was heated to ca 55°-60° C. for 30 minutes to give a solution which was clarified by filtration and the lines washed into the crystallisation vessel with THF/Water (7:3, 2 vol). The resultant solution was heated to reflux and tetrahydrofuran (9 vol, 95% w/w azeotrope with water) was distilled off at atmospheric pressure.

The solution was seeded with N-{3-Chloro-4-[(3-fluorobenzyl)oxy]phenyl}-6-[5-({[2-(methanesulphonyl)ethyl]amino}methyl)-2-furyl]-4-quinazolinamine ditosylate monohydrate (0.002 wt). Once the crystallisation was established water (6 vol) was added while maintaining the reaction temperature above 55° C. The mixture was cooled to 5°-15° C. over ca 2 hours. The solid was collected by filtration, washed with tetrahydrofuran/water (3:7 ratio, 2×2 vol) and dried in vacuo. 45° C. to give N-{3-Chloro-4-[(3-fluorobenzyl)oxy]phenyl}-6-[5-({[2-(methanesulphonyl)ethyl]amino}methyl)-2-furyl]-4-quinazolinamine ditosylate monohydrate as a bright yellow crystalline solid.

### Example 2

### N-{3-chloro-4-[(3-fluorobenzyl)oxy]phenyl}-6-(5-{[2-(methylsulfonyl)ethoxy]methyl}-2-furyl)-4-quinazolinamine

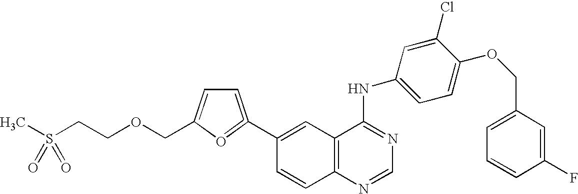

Prepared according to Procedure 0 of WO 01/04111 (referred to above) utilizing 3-[5-(4-{3-chloro-4-[(3-fluorobenzyl)oxy]anilino}-6-quinazolinyl)-2-furyl]-2-methen alcohol (66.8 mg, 0.141 mmol), methyl vinyl sulfone (0.015 mL, 0.169 mmol) and sodium hydride (60% in mineral oil, 0.7 mg, 0.017 mmol) in DMF (3 mL) to provide the title compound (51 mg) after purification by chromatography. ¹H NMR 400 MHz (DMSO-d₆) 9.95 (1H, s), 8.74 (1H, s), 8.50 (1H, s), 8.11 (1H, d, J=8.8 Hz), 7.96 (1H, s), 7.76-7.68 (2H, m), 7.41 (1H, m), 7.29-7.22 (3H, m), 7.11 (1H, m), 7.06 (1H, d, J=2.8 Hz), 6.65 (1H, d, J=2.8 Hz), 5.21 (2H, s), 4.55 (2H, s), 3.81 (2H, t), 3.37 (2H, t), 2.94 (3H, s). LC/MS m/z 582 (M+H⁺).

### Example 3

### 2-{{[5-(4-{3-chloro-4-[(3-fluorobenzyl)oxyjanilino}-6-quinazolinyl)-2-furyl]methyl}[2-(methylsulfonyl)ethyl]amino}acetonitrile

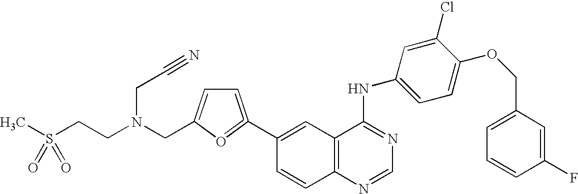

(4(3-Fluorobenzyloxy)-3-chlorophenyl)-(6-(2-((2-methanesulphonyl-ethylamino)-ethyl)-furan-2-yl)-quinazolin-4-yl)-amine (116 mg, 0.2 mmol), chloroacetonitrile (0.014 mL, 0.22 mmol) and diisopropyl ethyl amine (0.07 mL, 0.2 mmol) were mixed, as outlined in Procedure P of WO 01/04111, to provide the title compound (110 mg). ¹H NMR 400 MHz (DMSO-d₆) 9.84 (1H, s), 8.69 (1H, s), 8.50 (1H, s), 8.10 (1H, d, J=8.8 Hz), 7.96 (1H, d, J=2.4 Hz), 7.76 (1H, d, J=8.8 Hz), 7.68 (1 H, m), 7.42 (1H, m), 7.29-7.22 (3H, m), 7.13 (1H, m), 7.03 (1H, d, J=3.6 Hz), 6.59 (1H, d, J=3.6 Hz), 5.22 (2H, s), 3.84 (2H, s), 3.81 (2H, s), 3.37 (2H, t), 2.98 (3H, s), 2.96 (2H, t). LC/MS m/z 620 (M+H⁺).

### Example 4

### (4-(3-Fluorobenzyloxy)-3-chlorophenyl)-(6-(2-((2-iso-propyl-sulphonyl-ethylamino)-methyl)-furan-2-yl)-quinazolin-4-yl)-amine

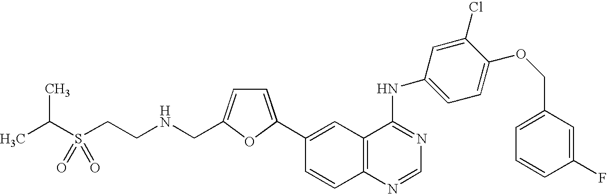

The title compound and its hydrochloride salt are prepared according to Procedure D of WO 01/047111 (page 97), utilizing 5-(4-[4-(3-fluoro-benzyloxy)-3-chloroanilino]-6-quinazolinyl}-2-furaldehyde (0.317 mmol, 0.15 g) Isopropylsulfonylethyl amine hydrochloride salt (0.475 mmol, 0.105 g) in the presence of Et₃N (0.95 mmol, 0.13 mL) and NaBH₄ (1.1 mmol, 0.041 g) in THF/MeOH. ¹H NMR (DMSO-d₆) 11.74 (bs, 1H); 9.90 (bs, 2H); 9.63 (s, 1H); 8.91 (s, 1H); 8.42 (d, 1H); 8.04 (m, 1H); 7.95 (d, 1H); 7.81 (d, 1H); 7.47 (m, 1H); 7.37-7.28 (m, 4H); 7.18 (m, 1H); 6.83 (m, 1H); 5.29 (s, 2H); 4.45 (s, 2H); 3.72-3.39 (m, 5H); 1.26 (d, 6H). Electrospray MS 609.

### Example 5

### N4-(1-Benzyl-1H-indazol-5-yl)-N6,N6-dimethyl-pyrido[3,4-d]pyrimidine-4,6-diamine

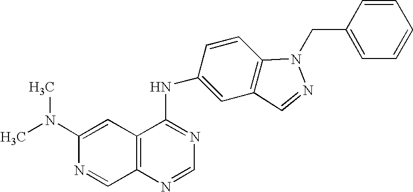

A stirred solution of (1-benzyl-1H-indazol-5-yl)-(6-chloro-pyrido[3,4-d]pyrimidin-4-yl)-amine (0.5 g) in 33% aqueous dimethylamine (5 ml) was heated at 130° C. in a reacti-vial for 17 hr. The cooled mixture was dissolved in chloroform, absorbed onto silica and chromatographed to give the title compound (Procedure C: Col 20, lines 10-16 of U.S. Pat. No. 6,174,889) as a yellow solid; δH [²H₆]-DMSO 9.00(1H,s), 8.51(1H,s), 8.09(2H,d), 7.55(1H,dd), 7.25(7H,m), 6.39(1H,m), 5.60(2H,s) 3.20 (6H,s); m/z (M+1)⁺ 396.

Examples 6-9 recite the preparation of specific cRaf-1 inhibitors useful in the present invention.

### Example 6

### Preparation of 3-(3,5-Dibromo-4-hydroxy-benzylidene)-5-iodo-1,3-dihydro-indol-2-one

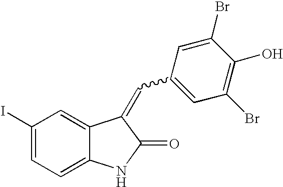

The title compound was prepared according to the procedures of U.S. Pat. No. 6,268,391 col. 70, line 25 to col. 77, line 2 and of Lackey et al, Bioorg. Med. Chem Lett., 10 (2000) 223-26.

### Example 7

### 3-(3,5-Dibromo-4-hydroxy-benzylidene)-5-pyrid-3-yl-1,3-dihydro-indol-2-one

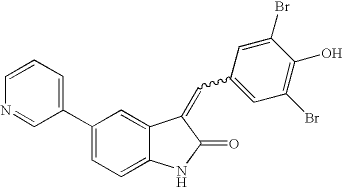

### (a) preparation of 5-pyrid-3-yl-1,3-dihydro-indol-2-one

A mixture of 0.736 g (2 mmol) of 3-tributyltin pyridine, 0.259 g (1 mmol) of 5-iodo-oxindole, 0.497 g (3 mmol) of tetraethyl ammonium chloride, and 0.035 g (0.05 mmol) of bis(triphenylphospine)palladium (II) chloride in 4 ml of acetonitrile was heated to reflux for 24 hrs. After cooling to ambient temperature the mixture was diluted with 20 ml of CHCl₃ and 50 ml of 10% potassium fluoride solution (aq) was added. Filtered the mixture through a 1 inch pad of celite and separated the layers. The organic layer was concentrated in vacuo, and the residue was chromatographed on silica gel (EtOAc/MeOH 5%) to afford 5-pyrid-3-yl-1,3-dihydro-indol-2-one as white solid (0.033 g, 16%): ¹H NMR (DMSO-d₆): δ 10.51 (s, 1H); 8.83 (d, J=2.2, 1H); 8.51 (dd, J₁=1.3, J₂=4.6, 1H); 8.02-7.97 (m, 1H); 7.59 (s, 1H); 7.54 (d, J=8.1, 1H); 7.44 (dd, J₁=4.7, J₂=7.9, 1H); 6.93 (d, J=8.1, 1H); 3.55 (s, 2H). APCI-MS: m/z 211 (m+H)⁺.

### (b) Preparation of 3-(3,5-Dibromo-4-hydroxy-benzylidene)-5-pyrid-3-yl-1,3-dihydro-indol-2-one

The title compound was prepared in an identical manner to example 2 of U.S. Pat. No. 6,268,391, except that 5-pyrid-3-yl-1,3-dihydro-indol-2-one was used in place of 5-(2-methyl-thiazol-4-yl)-1,3-dihydro-indol-2-one. ¹H NMR (DMSO-d₆): δ 10.93 (s, 1H); 9.13 (s, 1H); 8.81 (s, 2H); 8.8-8.7 (m, 1H); 8.6-8.5 (m, 1H); 8.23 (s, 1H); 7.94 (s, 1H); 7.9-7.8 (m, 1H); 7.72 (d, J=8, 1H); 7.02 (d, J=8, 1H). APCI-MS: m/471 (m-H)⁻.

### Example 8

### 3-(3,5-Dibromo-4-hydroxy-benzylidene)-5-(3-methyl-butanoyl)-1,3-dihydro-indol-2-one

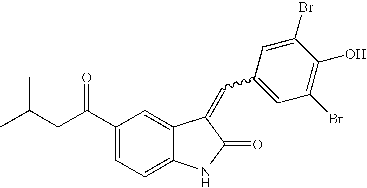

The title compound was prepared according to the procedures of U.S. Pat. No. 6,268,391 col. 70, line 25 to col. 77, line 2 and of Lackey et al, Bioorg. Med. Chem Lett., 10 (2000) 223-26.

### Example 9

### 3-(3,5-Dibromo-4-hydroxy-benzylidene)-5-(pyridine-4-carbonyl)-1,3-dihydro-indol-2-one

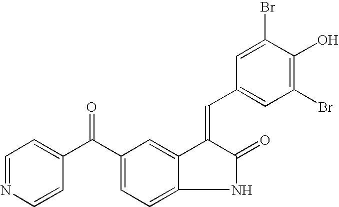

The title compound was prepared according to the procedures of U.S. Pat. No. 6,269,391 col. 70, line 25 to col. 77, line 2 and of Lackey et al, Bioorg. Med. Chem Lett., 10 (2000) 223-26.

Examples 10-16 recite the preparation of specific bRaf inhibitors useful in the present invention.

### Example 10

### 5-{2-[4-(3-Dimethylamino-propyloxy)-phenyl]-5-pyridin-4-yl-1H-imidazol-4-yl}-indan-1-one

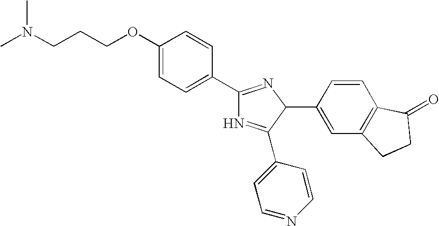

### (a) 5-Bromo-indan-1-one O-methyl-oxime

To a solution of 5-bromo-indanone (100 g, 0.474 mol) in ethanol (650 ml) under argon was added methoxylamine hydrochloride (198 g, 2.38 mol) and pyridine (125 ml). The mixture was refluxed for 2.5 hours, cooled to room temperature and poured into saturated aqueous sodium hydrogen carbonate solution. The mixture was then extracted with ethyl acetate and the organic phase dried (sodium sulphate) and then concentrated in vacuo. The crude material was recrystallised from isopropanol to furnish the title compound, (110 g, 97%), as a brown solid; ¹H NMR (CDCl₃) 67.52 (1H, d, J=8.3 Hz), 7.43 (1H, d, J=1 Hz), 7.35 (1H, dd, J 8.3, 1 Hz), 3.97 (3H, s), 2.99 (2H, m), 2.99 (2H, m), 2.85 (2H, m).

### (b) 1-Methoxyimino-indan-5-carbaldehyde

To a solution of the product of Step 1 (112 g, 0.46 mol) in THF (1500 ml) at −60° C. under argon, was added n-BuLi (325 ml, 0.52 mol) over 1 hour. After stirring at −60° C. for 1 hour a solution of DMF (39.7 ml) in THF (50 ml) was added dropwise over 1 hour. The reaction was stirred at −60° C. for 1 hour before being allowed to warm to room temperature. After 1 hour the reaction was quenched with saturated aqueous sodium hydrogen carbonate solution and extracted into ethyl acetate. The organic phase was then dried (sodium sulphate), concentrated in vacuo and the residue purified by silica gel chromatography, to give the title compound (57 g, 65%) as a yellow solid; ¹H NMR (CDCl₃) 10.0 1H, s), 7.83-7.73 (3H, m), 4.02 (3H, s), 3.10 (2H, m), 2.92 (2H, m).

### (c) 5-(1,2-Dihydroxy-2-pyridin-4-yl-ethyl)-indan-1-one-O-methyl-oxime

To a solution of 4-(tert-butyl-dimethyl-silanyloxymethyl)-pyridine [T. F. Gallagher et al; Bioorg. Med. Chem., 1997, 5, 49] (71.5 g, 0.32 mol) in THF (800 ml) at −50° C. under argon was added LDA (162 ml, 2M in heptane/THF/ethylbenzene, 0.324 mol) over 1 hour. The mixture was stirred at −40° C. for a further 1 hour before a solution of the product of Step 2 (55 g, 0.29 mol) in THF (600 ml) was added over 1 hour. The reaction was then allowed to warm to room temperature overnight before being quenched by the addition of saturated aqueous sodium hydrogen carbonate solution and then extracted into ethyl acetate. The organic phase was dried (sodium sulphate) and concentrated in vacuo to give a brown oil (125 g). The oil was then dissolved in THF (1500 ml), treated with TBAF (356 ml, 0.356 mol) and stirred for 1 hour. The reaction mixture was then evaporated and the residue partitioned between water and ethyl acetate. The organic phase was then dried (sodium sulphate) and concentrated to give the title compound (57 g, 64%) as a pale yellow solid which was used without further purification. ¹H NMR (CDCl₃) 8.38 (3H, m), 7.57 (2H, m), 7.12-6.99 (4H, m), 4.88 (1H, m), 4.66 (1H, m), 3.96 (3H, s), 2.93 (2H, m), 2.85 (2H, m).

### (d) 1-(1-Methoxyimino-indan-5-yl)-2-pyridin-4-yl-ethane-1,2-dione

To a mixture of DMSO (43 ml, 0.56 mol) and dichloromethane (800 ml) at −70° C. under argon, was added oxalyl chloride (43.2 g) and then a solution of the product of Step 3 (55 g, 0.185 mol) in a mixture of dichloromethane/DMSO (1000 ml/60 ml) over 2 hours at −60° C. After stirring for 2 hours at −60° C., triethylamine (154 ml) was added dropwise and the mixture then allowed to warm to room temperature overnight. The reaction mixture was then quenched with water, the organic phase separated then washed with water, dried (sodium sulphate) and concentrated to yield the title compound (51 g, 94%) as a yellow solid. ¹H NMR (CDCl₃) 8.87 (2H, d), 7.89-7.77 5H, m), 4.03 (3H, s), 3.09 (2H, m), 2.93 (2H, m).

### (e) 5-{2-[4-(3-Dimethylamino-propyloxy)-phenyl]-5-phenyl-1H-imidazol-4-yl}-indan-7-one O-methyl-oxime

A mixture of the product of Step 4 (0.3 g, 1.02 mmol), 4-(3-dimethylamino-propyloxy)-benzaldehyde (0.27 ml, 1.33 mmol) and ammonium acetate (0.785 g, 10.2 mmol) in acetic acid (10 ml) was heated to 100° C. for 1 hour. The reaction was then cooled to room temperature, poured into ice/0.880 ammonia solution and extracted with ethyl acetate. The organic extract was then dried (magnesium sulphate), concentrated in vacuo and the crude material purified by silica gel chromatography eluting with a 1:9:90 mixture of 0.88 ammonia solution:methanol:ethyl acetate to give the title compound (0.08 g, 16%) as a yellow solid; MS(AP+) m/e 483 [M+H]⁺.

### (f) 5-{2-[4-(3-Dimethylamino-propyloxy)-phenyl]-5-pyridin-4-yl-1H-imidazol-4-yl}-indan-1-one

A mixture of the product of Step 5 (0.07 g, 0.146 mmol) and 5M HCl (4 ml) in dioxan (3 ml) was heated to 100° C. for 1 hour. Acetone (3 ml) was then added and the heating continued for a further 1.5 hours before the mixture was cooled to room temperature, neutralised with 1M sodium hydroxide solution and extracted with ethyl acetate. The organic extract was then washed with water, dried (magnesium sulphate), concentrated in vacuo and the crude material purified by silica gel chromatography, eluting with a 2:18:80 mixture of 0.88 ammonia solution:methanol:ethyl acetate to give the title compound (0.035 g, 53%) as a yellow solid; MS(AP+) m/e 453 [M+H]⁺.

### Example 11

### 5-{2-[4-(3-Dimethylamino-propyloxy)-phenyl]-5-pyridin-4-yl-1H-imidazol-4-yl}-indan-1-one oxime

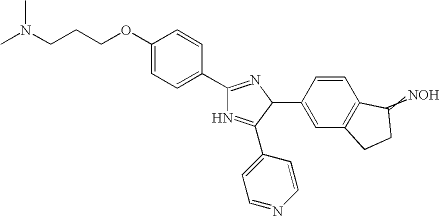

### (a) 5-{2-[4-(3-Dimethylamino-propyloxy)-phenyl]-5-pyridin-4-yl-1H-imidazol-4-yl}-indan-1-one oxime

To a solution of the product of Example 10, Step (f) (0.07 g, 0.155 mmol) in ethanol (3 ml) at 80° C. was added aqueous hydroxylamine (1.5 ml, 50% in water). After 30 minutes the mixture was cooled to room temperature and concentrated in vacuo to give the title compound, (0.072 g, 100%) as a yellow solid; MS(AP+) m/e 468 [M+H]⁺.

### Example 12

### 5-{2-[4-(2-Dimethylamino-ethoxy)-phenyl]-5-pyridin-4-yl-1H-imidazol-4-yl}-1-indanone

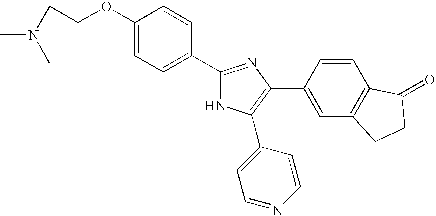

### (a) 5-{2-[4-(2-Dimethylamino-ethoxy)-phenyl]-5-pyridin-4-yl-1H-imidazol-4-yl}-indan-1-one-O-methyl-oxime

The title compound (0.19 g, 30%) was prepared from the product of Example 10, Step (d) and 4-(2-dimethylamino-ethoxy)-benzaldehyde [see WO 99/19293] as described in Example 10, Step (e); MS(AP+) m/e 468 [M+H]⁺.

### (b) 5-{2-[4-(2-Dimethylamino-ethoxy)-phenyl]-5-pyridin-4-yl-1H-imidazol-4-yl}-1-indanone

The title compound (0.313 g, 56%) was prepared from the product of Step 1 as described in Example 10 Step (f); MS(AP+) m/e 439 [M+H]⁺.

### Example 13

### 5-{2-[4-(2-Dimethylamino-ethoxy)-phenyl]-5-pyridin-4-yl-1H-imidazol-4-yl}-indan-1-one oxime

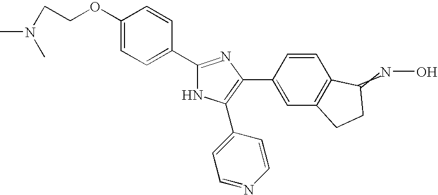

### (a) 5-{2-[4-(2-Dimethylamino-ethoxy)-phenyl]-5-pyridin-4-yl-1H-imidazol-4-yl}-indan-1-one oxime

The title compound (0.321 g, 100%) was prepared from the product of Example 12 Step (b) as described in Example 11 Step (a); MS(AP+) m/e 454 [M+H]⁺.

### Example 14

### 5-(5-Piperidin-4-yl-2-pyridin-4-yl-furan-3-yl)-indan-1-one oxime

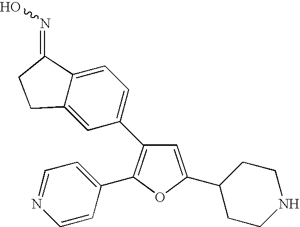

### (a) 1-Methoxyimino-indan-5-carbaldehyde

A solution of the product of Description 1 in WO 03/0228840 (112 g, 0.46 mol) in THF (1500 ml) at −60° C. under argon was treated with n-BuLi (325 ml, 1.6M in hexanes, 0.52 mol) over 1 hour. After stirring at −60° C. for 1 hour a solution of DMF (39.7 ml) in THF (50 ml) was added dropwise over 1 hour. The reaction was stirred at −60° C. for a further 1 hour before being allowed to warm to room temperature. The reaction was quenched with saturated aqueous sodium hydrogen carbonate solution and extracted into ethyl acetate. The organic phase was separated, dried, concentrated in vacuo and the residue purified by silica gel chromatography to give the title compound (57 g, 65%); ¹H NMR (CDCl₃) 10.0(1H,s), 7.83-7.73 (3H, m), 4.02 (3H, s), 3.10 (2H, m), 2.92 (2H, m).

### (b) 4-[(E)-3-(1-Methoxyimino-indan-5-yl)-allanoyl]-piperidine-1-carboxylic acid benzyl ester

A mixture of the product of (a) (4.8 g, 25 mmol), sodium methoxide (1.35 g, 25 mmol) and 4-acetyl-piperidine-1-carboxylic acid benzyl ester (6.49, 25 mmol) (WO97/05877) in methanol (100 ml) was heated at reflux for 8 hours. After cooling to room temperature the solution was concentrated in vacuo and the residue diluted with ethyl acetate and water. The organic phase was washed with water and brine, dried and concentrated in vacuo and the residue purified by silica gel chromatography to give the title compound (6.92 g, 64%); MS(ES+) m/e 433 [M+H]⁺.

### (c) 4-[3-(1-Methoxyimino-indan-5-yl)-4-oxo-4-pyridin-4-yl-butanoyl]-piperidine-1-carboxylic acid benzyl ester

A solution of sodium cyanide (240 mg, 4.8 mmol) in DMF (15 ml) was treated with a solution of pyridine-4-carbaldehyde (1.71 g, 16 mmol) in DMF (25 ml). After 15 minutes a solution of the product of (b) (6.92 g, 16 mmol) in DMF (20 ml) was added dropwise. After stirring at room temperature for 18 hours the mixture was diluted with saturated sodium bicarbonate solution and ethyl acetate. The organic phase was washed with water and brine, dried and concentrated in vacuo and the residue purified by silica gel chromatography to give the title compound (5.6 g, 65%); MS(ES+) m/e 540 [M+H]⁺.

### (d) 5-(5-Piperidin-4-yl-2-pyridin-4-yl-furan-3-yl)-indan-1-one O-methyl-oxime

The product of (c) 3 (3.0 g, 5.5 mmol) was added to a stirred suspension of phosphorus pentoxide (8 g) in dry methane sulphonic acid (50 ml). After stirring at room temperature for 4 hours the reaction mixture was cautiously poured into a stirred solution of ice cold 50% aqueous sodium hydroxide (final pH 10). The mixture was extracted with chloroform, washed with water and brine, dried and concentrated in vacuo and the residue purified by silica gel chromatography to give the title compound (0.66 g, 32%); MS(ES+) m/e 388 [M+H]+.

### (e) 5-(5-Piperidin-4-yl-2-pyridin-4-yl-furan-3-yl)-indan-1-one

The title compound was prepared from the product of (d) using the method described in Example 1 Step 2 of WO 03/022840; MS(ES+) m/e 359 [M+H]+.

### (f) 5-(5-Piperidin-4-yl-2-pyridin-4-yl-furan-3-yl)-indan-1-one oxime

The title compound was prepared from the product of (e) using the method described in Example 1 Step 3 of WO 03/022840; MS(ES+) m/e 374 [M+H]+.

### Example 15

### 5-{5-[1-(2-Methoxyethyl)-piperidin-4-yl]-2-pyridin-4-yl-furan-3-yl}-indan-1-one oxime

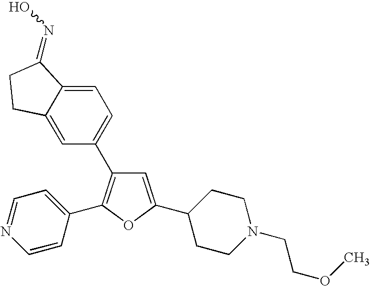

A mixture of the product of Example 14 (0.149 g, 0.4 mmol), methoxy-acetaldehyde (0.037 g, 0.5 mmol) (E. M. Acton et al, J. Med. Chem., 1986, 29, 2074) and polymer bound trimethylammonium cyanoborohydride (200 mg, 0.8 mmol, 4 mmol/g) in methanol (5 ml) containing acetic acid (0.2 ml) was stirred at room temperature for 24 hours. The reaction mixture was then filtered and the resin washed with methanol. The filtrate was concentrated in vacuo and the residue purified by silica gel chromatography eluting with a 1:9:90 mixture of 0.880 ammonia solution:ethanol:chloroform to give the title compound (0.103 g, 60%); MS(ES+) m/e 432 [M+H]⁺.

### Example 16

### 5-(5-{1-[2-(4-Chloro-phenoxy)-ethyl]-piperidin-4-yl}-2-pyridin-4-yl-furan-3-yl)-indan-1-one oxime

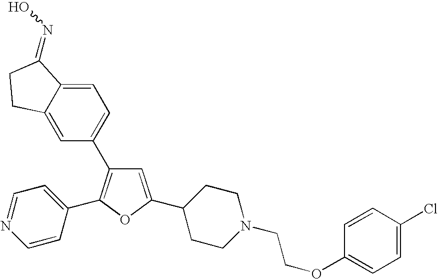

### (a) 5-(5-{1-[2-(4-Chloro-phenoxy)-ethyl]-piperidin-4-yl}-2-pyridin-4-yl-furan-3-yl)-indan-1-one

The title compound (0.140 g, 65%) was prepared from the product of Example 14 (e) and (4-chlorophenoxy)acetaldehyde (Maguire et al, J. Chem. Soc., 1954, 3669) using the method of Example 32; MS(ES+) m/e 513 [M+H]⁺.

### Step 2: 5-(5-{1-[2-(4-Chloro-phenoxy)-ethyl]-piperidin-4-yl}-2-pyridin-4-yl-furan-3-yl)-indan-1-one oxime

The title compound (0.115 g, 88%) was prepared from the product of (a) using the method of Example 1, Step 3 of WO 03/022840; MS(ES+) m/e 528 [M+H]⁺.

### Example 17

### Methods

Cell Lines

The HB4a cell line was derived from human mammary luminal tissue. HB4a-erbB2 and HB4a-ras cell lines were generated by overexpression of erbB2 and mutant Ha-(Val-12)-ras in HB4a cell line. The S1 cell line was established by sub-cloning ErbB-2 transfected HB4a (see Harris R A, Eichholtz T J, Hiles J D, Page M J and O'Hare M J. (1999). Int J. Cancer. 80, 477, 484.)

The PANC-1 and CFPANC-1 pancreatic cancer cell lines were obtained from ATCC. Both cell lines are k-Ras hetero mutant. PANC-1 (G12D/G) was initiated from a pancreatic carcinoma of ductal origin and CFPANC-1 (G12V/G) is a ductal pancreatic adenocarcinoma cell line. Both cell lines express EGFR and erbB2.

Cell Cultures

HB4a cells grew in RPMI 1640 supplemented with L-glutamine, 10% FBS (Hyclone), 10 ug/ml hydrocortisone, and 5 ug/ml insulin. S1 and ras transfected cells were cultured in RPMI 1640 supplemented with L-glutamine, 10% FBS and 50 μg/ml hygromycin. Cell cultures were maintained in a humidified atmosphere of 5% CO₂ at 37° C.

PANC-1 cells grew in DMDM (Dulbecco's modified Eagle's medium) supplemented with 4 mM L-glutamine, 15% FBS (Hyclone), 4.5 g/L glucose and 1.5 g/L sodium bicarbonate.

CFPAC-1 cells were cultured in IMDM (Iscove's modified Dulbecco's medium) supplemented with 10% FBS. Cell cultures were maintained in a humidified atmosphere of 5% CO₂ at 37° C.

Inhibitor Solution Preparation

(a) The dual EGFR/erbB-2 inhibitor N-{3-Chloro-4-[(3-fluorobenzyl)oxy]phenyl}-6-[5-({[2-(methanesulphonyl)ethyl]amino}methyl)-2-furyl]-4-quinazolinamine and the cRaf-1 inhbitor of 3-(3,5-Dibromo-4-hydroxy-benzylidene)-5-iodo-1,3-dihydro-indol-2-one were dissolved in 100% DMSO to form solutions of 1, 5, 10, and 20 μM concentrations and tested on cells in growth medium for 3 continuous days. 0.2% DMSO in growth medium was the control for compound free cells.

(b) The dual EGFR/erbB-2 inhibitor N-{3-Chloro-4-[(3-fluorobenzyl)oxy]phenyl}-6-[5-({[2-(methanesulphonyl)ethyl]amino}methyl)-2-furyl]-4-quinazolinamine and the bRaf-1 inhbitors of Examples 13 and 14 were dissolved in 100% DMSO to form solutions of 5, 10 uM concentrations and tested on cells in growth medium for 3 or 4 continuous days. 0.1% DMSO in growth medium was the control for compound free cells.
GW2016 is N-{3-Chloro-4-[(3-fluorobenzyl)oxy]phenyl}-6-[5-({[2-(methane sulphonyl)ethyl]amino}methyl)-2-furyl]-4-quinazolinamine; and
GW5074 is 3-(3,5-Dibromo-4-hydroxy-benzylidene)-S-iodo-1,3-dihydro-indol-2-one.

Compound of Example 13 is 5-{2-[4-(2-dimethylamino-ethoxy)-phenyl]-5-pyridin-4-yl-1H-imidazol-4-yl}-indan-1-one oxime.

Compound of Example 14 is 5-(5-piperidin-4-yl-2-pyridin-4-yl-furan-3-yl)-indan-1-one oxime.

Cell Cycle Analysis

Cells were harvested and fixed with 70% ethanol in PBS. Cell pellets were then resuspended in 0.5 ml PBS containing propidium iodide (50 ug/ml) and DNase-free RNase (100 ug/ml). Cell cycle analysis was performed using a BD Flow Cytometer (Becton Dickinson, San Jose, Calif., USA).

For Mortality from exposure to the compounds, Trypan blue exclusion was used as the criterion for cell survival.

All experiments were derived from assays of 3 parallel samples.

Western Blots

Whole cell extracts were prepared by scraping cells off petri dishes, washing the cell pellet twice in PBS, and then resuspending the pellet in two-packed-cell volumes of RIPA buffer (150 mM NaCl, 50 mM Tris-HCl, pH 7.5, 0.25% deoxycholate, 1% NP-40, 5 mM sodium orthovanadate, 2 mM sodium fluoride and a protease inhibitor cocktail). Protein concentrations were determined using a modification of the Bradford method (Bio-Rad Laboratory). Steady state levels of total erbB-2, EGFR, Ras, Erk1/2, pErk1/2 and pTyr protein were assessed by Western blot which was carried out as follows: equal amounts of proteins was resolved by 7.5% SDS polyacrylamide gel electrophoresis under reducing conditions. Proteins were transferred to Immobilon-P or nitrocellulose membranes. Efficiency and equal loading of proteins was evaluated by Ponceau S staining. Membranes were blocked for 1 hr in TBS (25 mM Tris-HCl, pH 7.4, 150 mM NaCl, 2.7 mM KCl) containing 4% (w/v) lowfat milk or 3% BSA (w/v). Membranes were then probed with specific antibodies recognizing target proteins. Proteins were visualized with the SuperSignal West Femto Maximum sensitivity Substrate Kit (Pierce).

Expression of ErbB2 in Parental and ras-Transfected HB4a Cells

To determine ErbB2 expression in ras-transfected HB4a cells, compared to the parental line a Taqman assay was carried out as described following. Isolation of the RNA and the cDNA template preparation was performed first. The plus/minus RT experiment with RNA indicated that there was no genomic DNA contamination in this RNA template. Gels of the RNA showed no degradation of RNA. The 18S normalization experiment with the cDNA template indicated that all samples were of equal cDNA concentration. ErbB2 primers were as follows:

Accession number: M11730

| | |
|---|---|
| ErbB2-2661F | GGATGTGCGGCTCGTACAC |
| ErbB2-2735R | GTAATTTTGACATGGTTGGGACTCT |
| ErbB2-2684T | ACTTGGCCGCTCGGAACGTGC |

There were two RNA isolations from HB4a cells (#1 and 2) and two RNA isolations from HB4a-ras cells (#3 and 4). Each of these samples was run in quadruplicate in the Taqman assays. The ras-transfected cell lines expressed 16-fold less ErbB2 than the parental HB4a cells. The regulation is transcriptional.
Cell Cultures and Drug Treatment

HB4a cells growing in RPMI 1640 supplemented with L-glutamine, 10% FBS (Hyclone), 10 ug/ml hydrocortisone, and 5 μg/ml insulin. S1 cells and HB4a-ras were cultured in RPMI 1640 supplemented with L-glutamine, 10% FBS and 50 μg/ml hygromycin. Cell cultures were maintained in a humidified atmosphere of 5% CO₂ at 37° C. For inhibitor treatment, cells were seeded at low density in the medium and then exposed for 72 hours to GW2016, GW5074 or combination of GW2016 and GW5074 at various concentrations as indicated in the Figures. For Cell Cycle Analysis, cells were harvested, washed twice in phosphate buffered saline (PBS) and fixed with 700/0 ethanol in PBS. Cell pellets were then resuspended in 0.5 ml PBS containing propidium iodide (50 ug/ml) and DNase-free RNase (100 ug/ml). Cell cycle analysis was performed using a BD Flow Cytometer (Becton Dickinson, San Jose, Calif., USA). For Mortality from exposure to the compounds Trypan blue exclusion was used as the criterion for cell survival. For western Blots, whole cell extracts were prepared by scraping cells off petri dishes, washing the cell pellet twice in PBS, and then lysed in RIPA buffer (150 mM NaCl, 50 mM Tris-HCl, pH 7.5, 0.25% (w/v) deoxycholate, 1% NP-40, 5 mM sodium orthovanadate, 2 mM sodium fluoride, and a protease inhibitor cocktail). Protein concentrations were determined using a modification of the Bradford method (Bio-Rad Laboratory). Steady state levels of erbB-2, EGFR, ras, total Erk1/2 and pTyr as well as activated Erk1/2 (p-Erk) were assessed by Western blot as following protocol equal amounts of proteins were resolved by either 7.5% SDS polyacrylamide gel electrophoresis under reducing conditions. Proteins were transferred to Immobilon-P or nitrocellulose membranes. Efficiency and equal loading of proteins was evaluated by Ponceau S staining. Membranes were blocked for 1 hr in TBS (25 mM Tris-HCl, pH 7.4, 150 mM NaCl, 2.7 mM KCl) containing 4% (w/v) lowfat milk or 3% BSA (w/v). Membranes were then probed with specific antibodies recognizing target proteins. Proteins were visualized with the SuperSignal West Femto Maximum sensitivity substrate kit (Pierce). Cell cycle analysis and Trypan blue exclusion were derived from assays of 3 parallel samples.

Cell Proliferation Assay

The colorimetric assay for the quantification of cell proliferation and cell viability was based on the cleavage of the tetrazolium salt WST-1 (4-[3-(4-Iodophenyl)-2-(4-nitrophenyl)-2H-5-tetrazolio]-1,3-benzene) (Roche Diagnostics Gmbh, Mannheim, Germany) by mitochondrial dehydrogenases in viable cells.

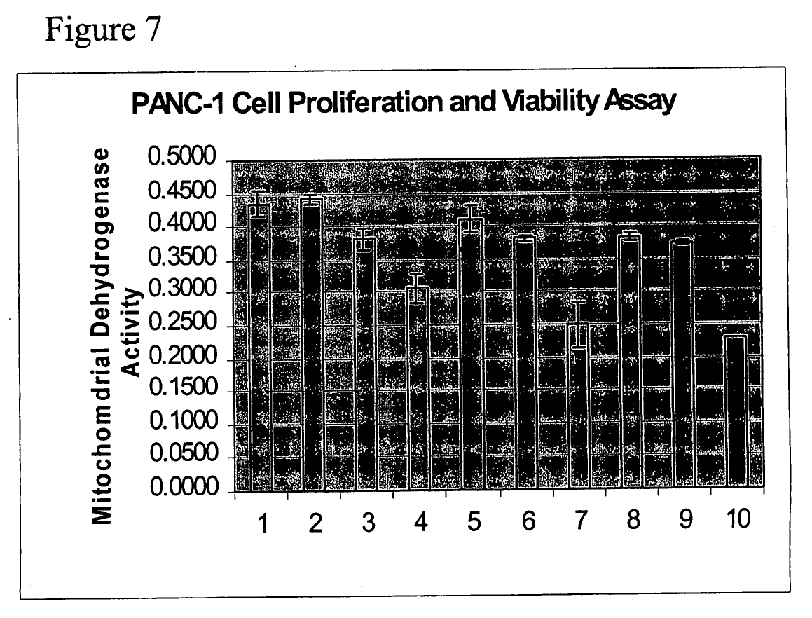

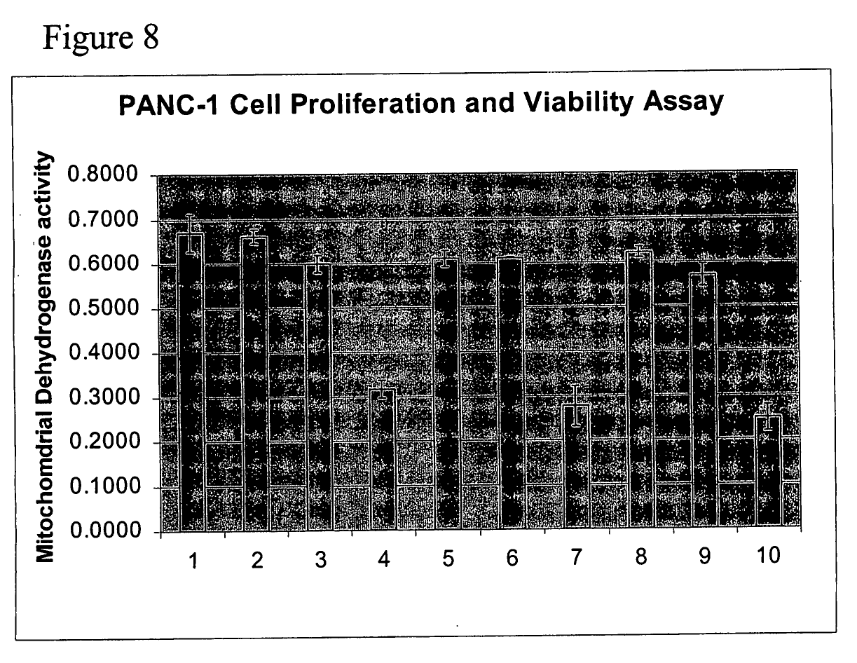

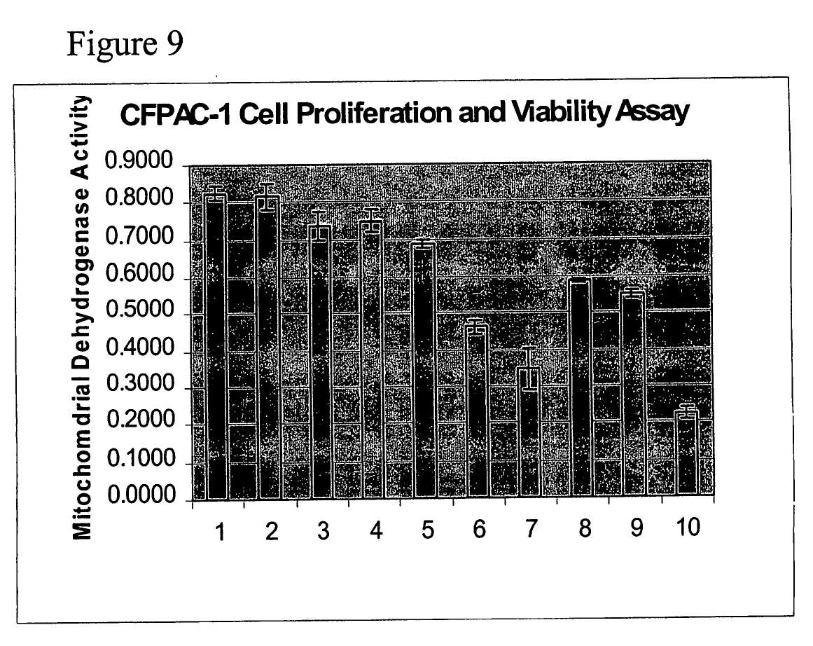

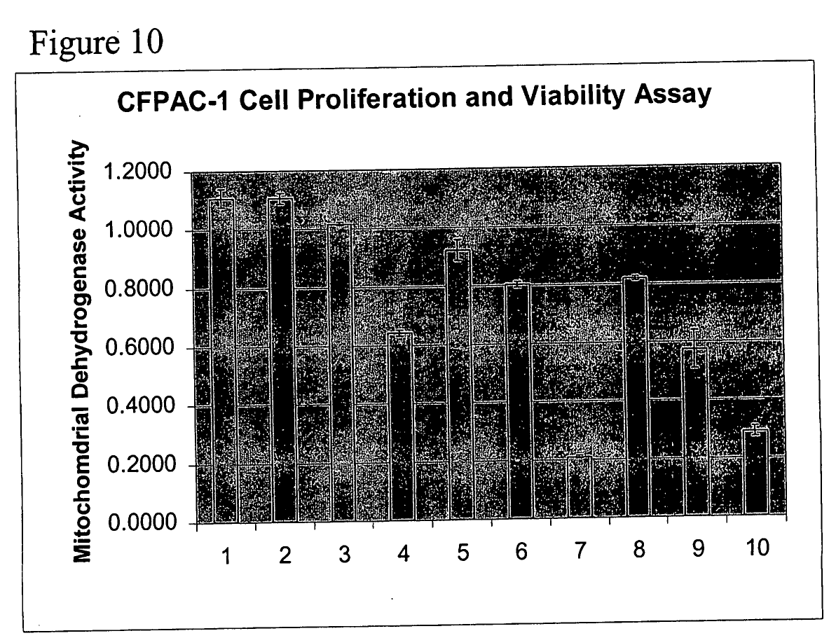

The cells were seeded at a concentration of 2,500/well in Microplates in a final volume of 100 ul with or without compounds as shown in the figures for 72 hour or 96 hour incubation in a humidified atmosphere of 5% CO₂ at 37° C. After the incubation, 10 ul/well of cell proliferation reagent WST-1 was added. A additional 2-3 hour was given before to measure the absorbance of the samples using a microplate reader at 480 nm. FIGS. A, B, C and D are showing the results of cell proliferation from PANC-1 and CFPANC-1. The cells were treated by GW2016 at 5 uM or 10 uM or the compound of Example 135 uM (B1) or the compound of Example 14 at 5 uM (B2) or a combination treatment of GW572016 and b-Raf inhibitors as indicated in the figures:
FIG. 7—PANC-1 cells for 72 hour compound treatment;
FIG. 8—PANC-1 cells for 96 hour compound treatment;
FIG. 9—CFPANC-1 cells for 72 hour treatment.
FIG. 10—CFPANC-1 for 96 hour treatment.
   Results

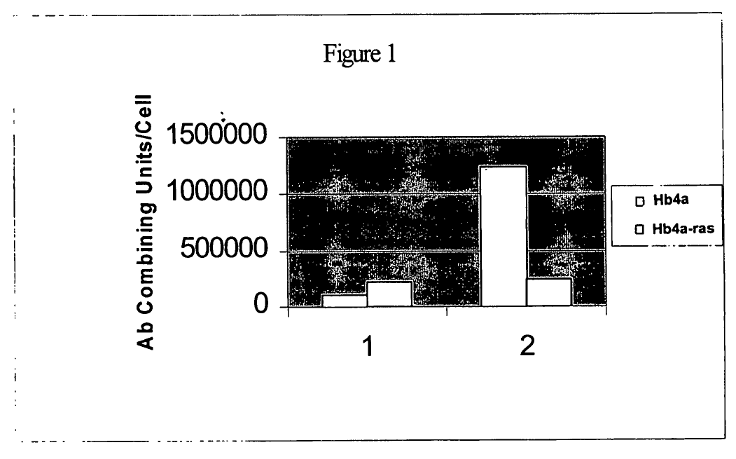

Experimental results are depicted in FIGS. 1 to 5 and 7-10.

FIG. 1 illustrates the comparison between erbB-2 and EGFR cell surface protein expression in the untransfected, parental HB4a cell line and an HB4a cell line which has been transfected with Ha-(Val.12)-ras. In the bar graph, on the horizontal, the 2 columns labeled “1” represent a quantification of EGFR and the columns labeled “2” represent a quantification of erbB-2 both in HB4a and HB4a-ras cells. The vertical scale represents antibody combining units per cell by a cell line linear scale. The left hand column of each pair, coded light gray, represents a counting of the respective kinase in HB4a cells and the right hand column of each pair, coded white, represents a counting of the respective kinase in HB4a-ras transfected cells. FIG. 1 shows a marked decrease in erbB-2 expression in the ras transfected HB4a cells. While not wishing to be bound by theory, the present inventors postulate that overexpression of ras in cells may down regulate erbB-2 expression in such cells.

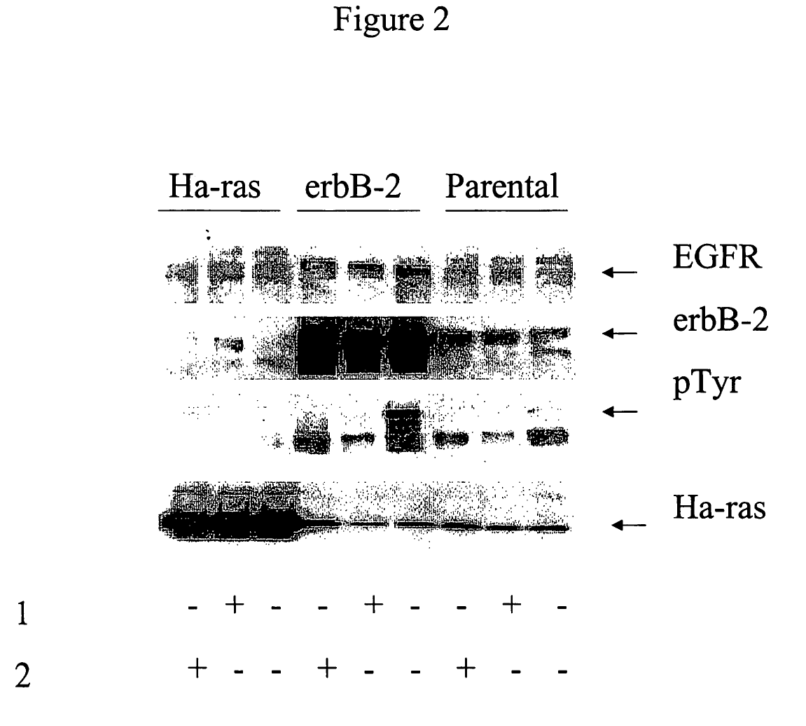

FIG. 2 shows results of a Western Blot study illustrating the effect of the dual EGFR/erbB-2 inhibitor GW2016 on an Ha-(Val12)-ras transfected Hb4a cell line, an erbB-2 transfected Hb4a cell line, and the parental Hb4a cell line. Expression of EGFR, erbB-2, pTyr, and Ha-ras is examined. The left three columns of the blot represent Ha-ras transfected HB4a cells, the middle three columns of the blot represent erbB-2 transfected HB4a cells, and the right three columns represent parental HB4a cells. Line 1 represents treatment of “+” marked columns with 1 μM GW2016/72 hours. Line 2 represents treatment of “+” marked columns with 5 μM GW2016/72 hours. The columns marked “−” were untreated. The ras transfected columns (left three columns) again show the postulated downregulation of erbB-2 in cells overexpressing ras. These columns show no or minimal erbB-2 expression, no phosphorylation on tyrosine residues, and no effect from GW2016 treatment. In contrast, the erbB-2 transfected cells (middle three columns) as expected depict erbB-2 expression, and subsequent reduction in tyrosine phosphorylation as a result of GW2016 treatment. In a like manner, parental cells (right three columns) depict erbB-2 expression, and subsequent reduction in tyrosine phosphorylation as a result of GW2016 treatment.

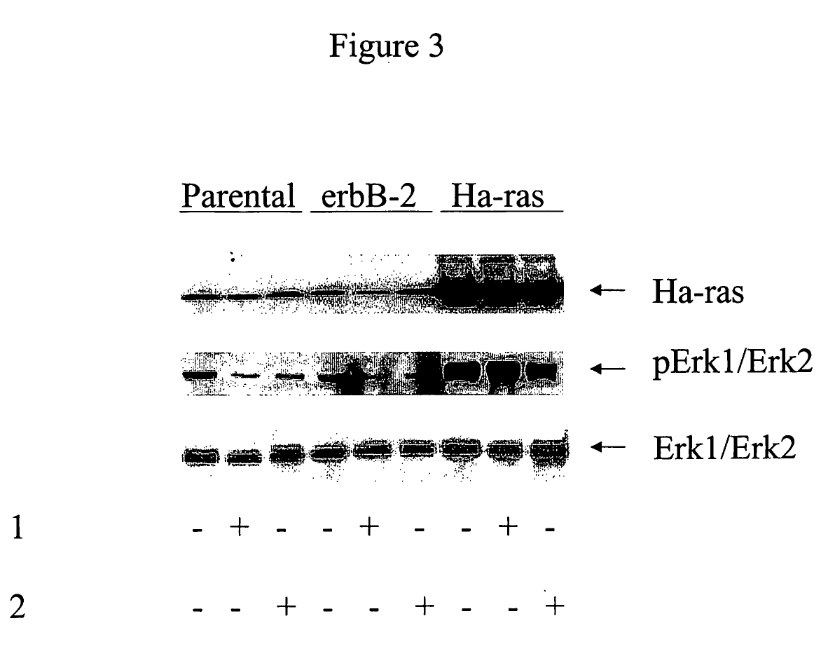

FIG. 3 shows results of a Western Blot study illustrating the effect of the dual EGFR/erbB-2 inhibitor GW2016 on the parental Hb4a cell line, an erbB-2 transfected Hb4a cell line, and an Ha-(Val12)-ras transfected Hb4a cell line, in regard to inhibition of MAPK Activation in such HB4a cell lines. Expression of Ha-ras, pERK1/Erk2, and Erk1/Erk2 is examined. The left three columns of the blot represent parental HB4a cells, the middle three columns of the blot represent erbB-2 transfected HB4a cells, and the right three columns represent Ha-(Val 12)-ras transfected HB4a cells. Line 1 represents treatment of “+” marked columns with 1 μM GW2016/72 hours. Line 2 represents treatment of “+” marked columns with 5 μM GW2016/72 hours. The columns marked “−” were untreated. The ras transfected columns (right three columns) show the effect of the postulated downregulation of erbB-2 in cells overexpressing Ha-ras. These columns show overexpression of Ha-ras and no effect from GW2016 treatment on the phosphorylation of Erk1/Erk2. In contrast, the erbB-2 transfected cells (middle three columns) as expected depict show a decrease in pErk1/Erk2 when treatment with GW2016 occurs. In a like manner, parental cells (left three columns) depict reduction in Erk1/Erk2 phosphorylation when GW2016 treatment is instituted.

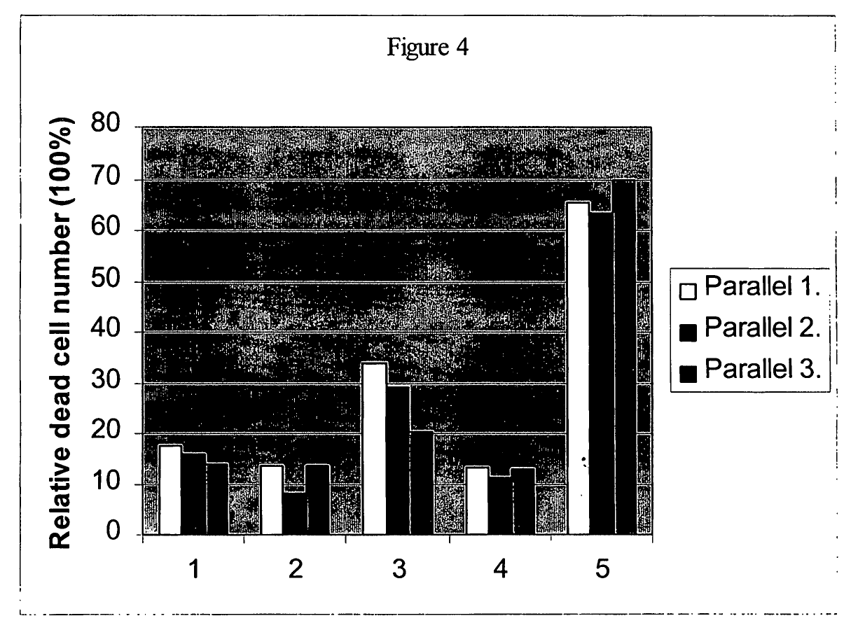

FIG. 4 depicts results showing cell mortality from exposure to GW2016 or GW5074 or GW2016+GW5074. The bar graph shows measurement of the percent of relative dead cells (vertical axis) for 5 treatments of HB4a cells transfected with Ha-(Val 12)-ras. Treatments 1-5 (labeled on the horizontal axis) were as follows:
1 control (non-treated cells)
2 cells treated with DMSO (solvent)
3 cells treated with GW 2016 (10 μM/72 hours)
4 cells treated with GW5074 (20 μM/72 hours)
5 cells treated with GW 2016 (10 μM/72 hours) and
GW5074 (10 μM/72 hours).

FIG. 4 illustrates a vastly increased HB4a-ras cell mortality with a cell treatment of both GW2016 and GW5074 (see 5), over treatment with either inhibitor alone (see 3 or 4).

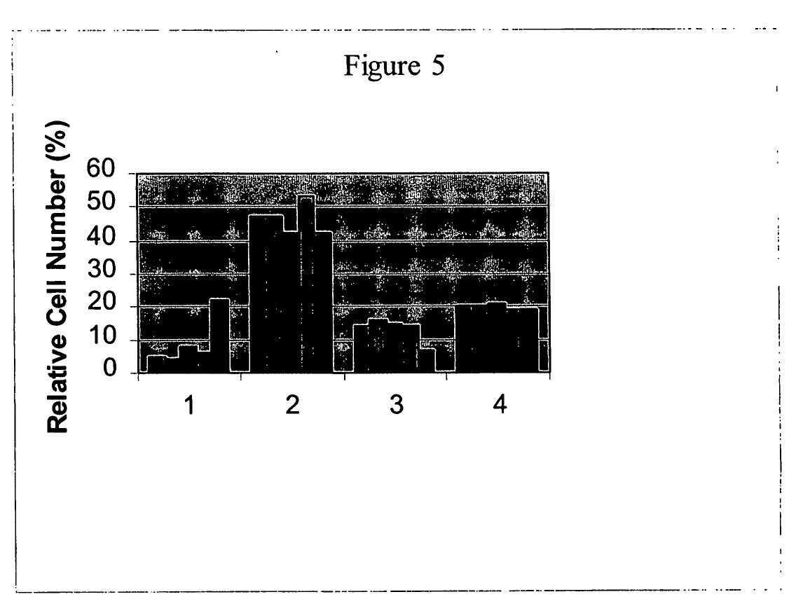

FIG. 5 depicts a bar chart showing a summary of cell cycle distribution analyses of cells exposed to GW2016 (10 μM/72 hours) or GW5074 (20 μM/72 hours) or GW2016 (10 μM/72 hours)+GW5074 (10 μM/72 hours). The bar graph shows measurement of the relative cell number (vertical axis) for cells at various points in the cell cycle and for apoptotic cells again for 5 treatments of HB4a cells transfected with Ha-(Val 12)-ras. The types of cells measured are labeled 1-4 (on the horizontal axis) were as follows:
1 apoptotic cells
2 cells in G₀/G₁ phase
3 cells in S phase
4 cells in G₂/M phase

Each type of labeled cells 1-4 has 5 columns which represents a treatment regimen coded from left to right for each set of 5 columns as follows:
Control−DMSO−GW2016−GW5074−GW2016+GW5074.

FIG. 5 also reveals that HB4a-ras cells subjected to treatment with a combination of GW2016 and GW5074 showed enhanced apoptosis over treatment with either inhibitor alone (see 1).

FIGS. 7-10 depicts a bar chart of the Mitochondrial Dehydrogenase Activity (O.D. 485) resulting from a Cell Proliferation and Viability Assay performed on PANC-1 (FIGS. 7-8) and CFPAC-1 (FIGS. 9-10) cells treated GW2016 and/or bRaf inhibitors B1 or B2 as indicated. Each numbered bar represents a treatment sample as follows:
1 Control
2 DMSO
3 GW2016 5 uM
4 GW2016 10 uM
5 B1 5 uM
6 GW2016 5 uM+B1 5 uM
7 GW2016 10 uM+B1 5 uM
8 B2 5 uM
9 GW2016 5 uM+B2 5 uM
10 GW2016 10 uM+B2 5 uM

1. A method of treating a susceptible cancer in a mammal, comprising: administering to said mammal therapeutically effective amounts of (i) a compound of formula (I) 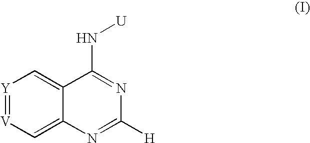
or a salt, solvate, physiologically functional derivative thereof,
wherein
Y is CR¹ and V is N:
or Y is CR¹ and V is CR²;
R¹ represents a group CH₃SO₂CH₂CH₂NHCH₂—Ar—, wherein Ar is selected from phenyl, furan, thiophene, Pyrrole and thiazole, each of which may optionally be substituted by one or two halo, C₁₋₄ alkyl or C₁₋₄ alkoxy groups:
R² is selected from the group comprising hydrogen, halo, hydroxy, C₁₋₄ alkyl, C₁₋₄ alkoxy, C₁₋₄ alkylamino and di[C₁₋₄ alkyl]amino:
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 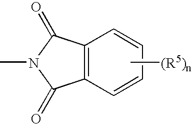
wherein each R⁵ is independently selected from halogen, C₁₋₄ alkyl and C₁₋₄ alkoxy; and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; and
(ii) at least one Raf and/or ras inhibitor.

2. (canceled)

3. A method of treating a susceptible cancer in a mammal, comprising: administering to said mammal therapeutically effective amounts of (i) a compound of formula (I) 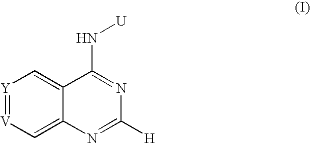
or a salt, solvate, physiologically functional derivative thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 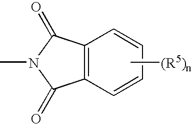
wherein each R⁵ is independently selected from halogen, C₁₋₄ alkyl and C₁₋₄ alkoxy; and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; and
(ii) a cRaf-1 inhibitor.

4. A method of treating a susceptible cancer in a mammal, comprising: administering to said mammal therapeutically effective amounts of (i) a compound of formula (III): 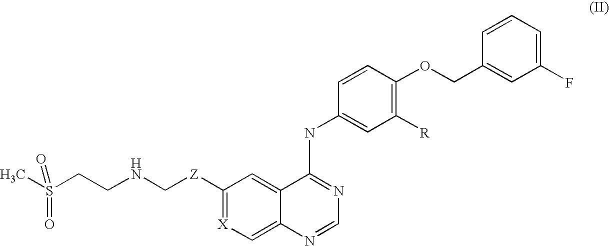
and salt or solvates thereof, wherein R is —Cl or —Br, X is CH, N, or CF, and Z is thiazole or furan; and
(ii) a cRaf-1 inhibitor.

5. A method of treating a susceptible cancer in a mammal, comprising: administering to said mammal therapeutically effective amounts of (i) a compound of formula (III): 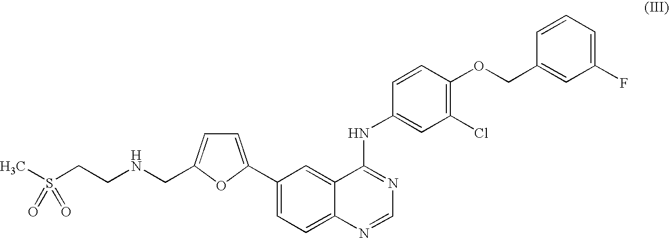
and salts or solvates thereof; and
(ii) a cRaf-1 inhibitor.

6. A cancer treatment combination, comprising:
therapeutically effective amounts of (i) compound of formula (I) 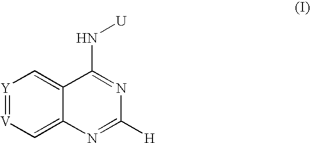
or a salt, solvate, physiologically functional derivative thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo. C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 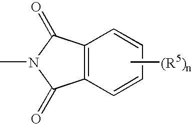
wherein each R⁵ is independently selected from halogen, C₁₋₄alkyl and C₁₋₄ alkoxy; and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N-(C₁₋₄ alkyl)carbamoyl, N,N-di(C₁₋₄ alkyl)carbamoyl, cyano, nitro and trifluoromethyl; and
(ii) at least one Raf and/or ras inhibitor.

7. (canceled)

8. A cancer treatment combination, comprising:
therapeutically effective amounts of (i) a compound of formula (I) 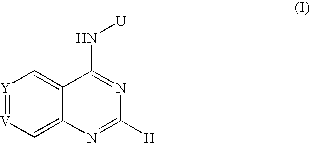
or a salt, solvate, or physiologically functional derivative thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 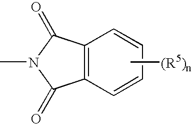
wherein each R⁵ is independently selected from halogen, C₁₋₄ alkyl and C₁₋₄ alkoxy; and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N-(C₁₋₄ alkyl)carbamoyl, N,N-di(C₁₋₄ alkyl)carbamoyl, cyano, nitro and trifluoromethyl; and
(ii) a cRaf-1 inhibitor.

9. A cancer treatment combination, comprising: therapeutically effective amounts of (i) a compound of formula (II): 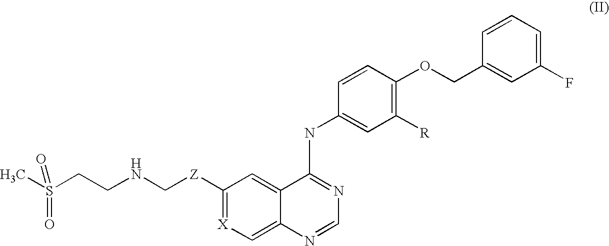
and salt or solvates thereof, wherein R is —Cl or —Br, X is CH, N, or CF, and Z is thiazole or furan; and
(ii) a cRaf-1 inhibitor.

10. A cancer treatment combination, comprising: therapeutically effective amounts of (i) a compound of formula (III): 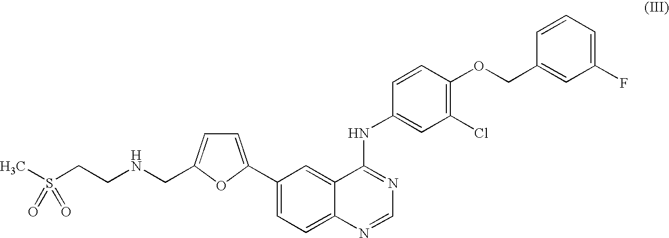
and salts or solvates thereof; and
(ii) a cRaf-1 inhibitor.

11-15. (canceled)

16. A method of treating a susceptible cancer in a mammal, comprising: administering to said mammal therapeutically effective amounts of (i) a compound of formula (I) 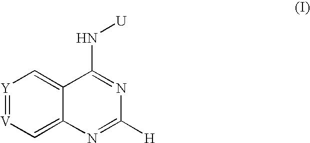
or a salt, solvate, physiologically functional derivative thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 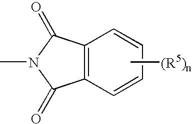
wherein each R⁵ is independently selected from halogen, C₁₋₄ alkyl and C₁₋₄ alkoxy; and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; and
(ii) a bRaf inhibitor.

17. A method of treating a susceptible cancer in a mammal, comprising: administering to said mammal therapeutically effective amounts of (i) a compound of formula (II): 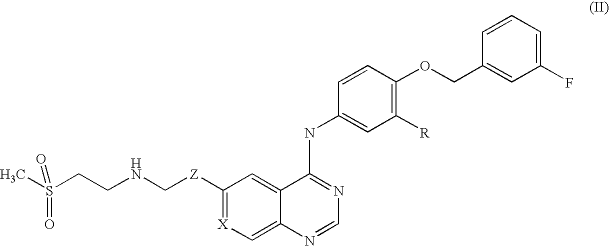
and salt or solvates thereof, wherein R is —Cl or —Br, X is CH, N, or CF, and Z is thiazole or furan; and
(ii) a bRaf inhibitor.

18. A method of treating a susceptible cancer in a mammal, comprising: administering to said mammal therapeutically effective amounts of (i) a compound of formula (III): 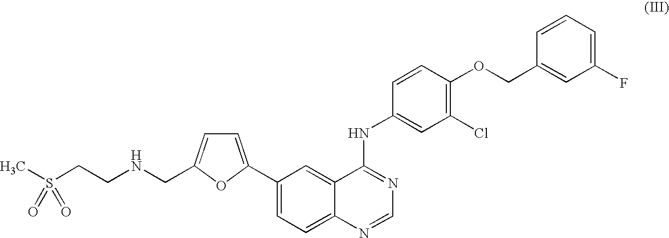
and salts or solvates thereof; and
(ii) a bRaf inhibitor.

19. (canceled)

20. A cancer treatment combination, comprising: therapeutically effective amounts of (i) a compound of formula (I) 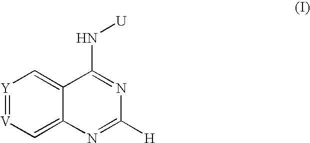
or a salt, solvate, or physiologically functional derivative thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 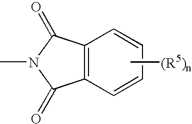
wherein each R⁵ is independently selected from halogen, C₁₋₄ alkyl and C₁₋₄ alkoxy; and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; and
(ii) a bRaf inhibitor.

21. A cancer treatment combination, comprising:
therapeutically effective amounts of (i) a compound of formula (II): 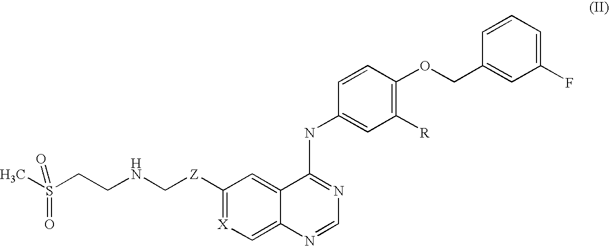
and salt or solvates thereof, wherein R is —Cl or —Br, X is CH, N, or CF, and Z is thiazole or furan; and
(ii) a bRaf inhibitor.

22. A cancer treatment combination, comprising: therapeutically effective amounts of (i) a compound of formula (III): 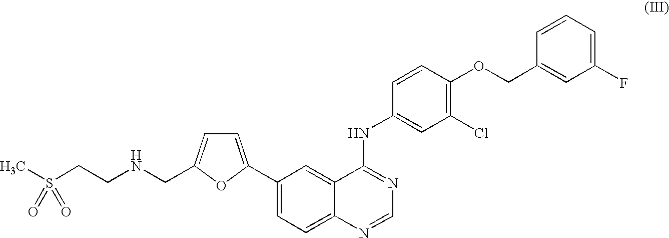
and salts or solvates thereof; and
(ii) a bRaf inhibitor.

23-24. (canceled)

25. The method of claim 1, wherein the compound of formula (I) is the monohydrate ditosylate salt of

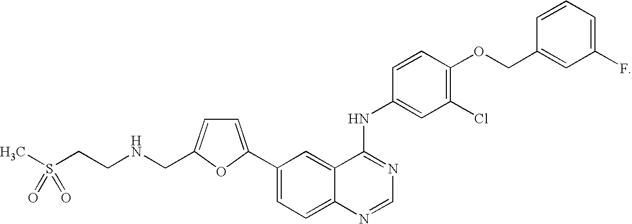

26. The cancer treatment combination of claim 6, wherein the compound of formula (I) is the monohydrate ditosylate salt

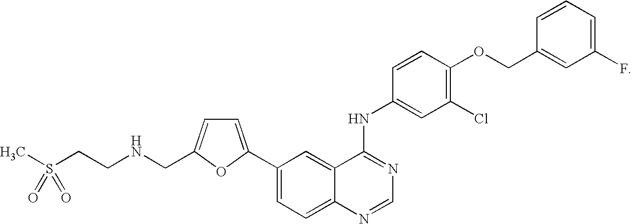

27. The method of claim 3, wherein the compound of formula (I) is the monohydrate ditosylate salt of

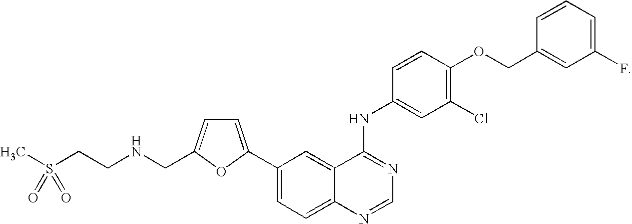

28. The cancer treatment combination of claim 8, wherein the compound of formula (I) is the monohydrate ditosylate salt

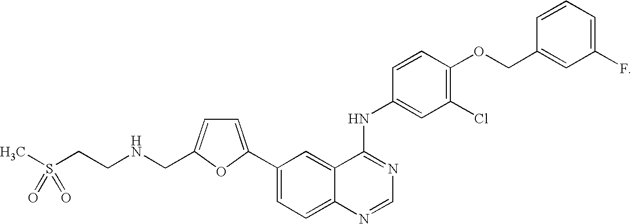

29. The method of claim 16, wherein the compound of formula (I) is the monohydrate ditosylate salt of

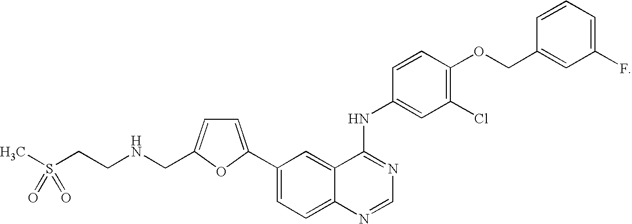

30. The method of claim 20, wherein the compound of formula (I) is the monohydrate ditosylate salt of [chemical structure]
